(19) United States
(12) Patent Application Publication
(10) Pub. No.: US 2010/0298359 A1
(43) Pub. Date: Nov. 25, 2010

(54) GAMMA SECRETASE MODULATORS

(76) Inventors: Xianhai Huang, Warren, NJ (US); Anandan Palani, Bridgewater, NJ (US); Jun Qin, Somerset, NJ (US); Robert Aslanian, Rockaway, NJ (US); Zhaoning Zhu, Plainsboro, NJ (US); William Greenlee, Teaneck, NJ (US)

Correspondence Address: MERCK;PATENT DEPARTMENT (K-6-1, 1990), 2000 GALLOPING HILL ROAD, KENILWORTH, NJ, 07033-0530, US

(21) Appl. No.: 12/746,313

(22) PCT Filed: Dec. 4, 2008
(86) PCT No.: PCT/US08/85520

§ 371 Date: Jul. 22, 2010

Related U.S. Application Data
(60) Provisional application No. 60/992,846, filed on Dec. 6, 2007.

(51) Int. Cl. A61K 31/517; C07D 401/10; A61K 31/4725; A61P 29/00; A61K 39/395; A61P 25/28; C07D 403/10

(52) U.S. Cl. 514/266.23; 546/148; 514/307; 424/172.1; 544/284

(57) ABSTRACT

This invention provides novel compounds that are modulators of gamma secretase. The compounds have the formula (I) wherein R² is a fused bicyclic ring of the formula (II). Also disclosed are methods of modulating gamma secretase activity and methods of treating Alzheimer's disease using the compounds of formula (I).

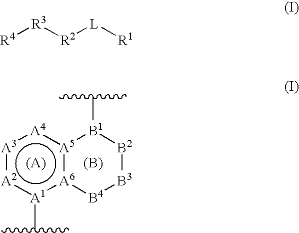

### REFERENCE TO RELATED APPLICATION

This application claims the benefit of U.S. Provisional Application Ser. No. 60/992,846 filed Dec. 6, 2007.

### FIELD OF THE INVENTION

The present invention relates to certain heterocyclic compounds useful as gamma secretase modulators, pharmaceutical compositions containing the compounds, and methods of treatment using the compounds and compositions to treat various diseases including central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

### BACKGROUND OF THE INVENTION

Alzheimer's disease is a disease characterized by degeneration and loss of neurons and also by the formation of senile plaques and neurofibrillary change. Presently, treatment of Alzheimer's disease is limited to symptomatic therapies with a symptom-improving agent represented by an acetylcholinesterase inhibitor, and the basic remedy which prevents progress of the disease has not been developed. A method of controlling the cause of onset of pathologic conditions needs to be developed for creation of the basic remedy of Alzheimer's disease.

Aβ protein, which is a metabolite of amyloid precursor protein (hereinafter referred to as APP), is considered to be greatly involved in degeneration and loss of neurons as well as onset of demential conditions (for example, see Klein W L, et al Proceeding National Academy of Science USA, Sep. 2, 2003, 100(18), p. 10417-22, suggest a molecular basis for reversible memory loss.

Nitsch R M, and 16 others, Antibodies against β-amyloid slow cognitive decline in Alzheimer's disease, Neuron, May 22, 2003, 38(4), p. 547-554) suggest that the main components of Aβ protein are Aβ40 consisting of 40 amino acids and Aβ42 having two additional amino acids at the C-terminal. The Aβ40 and Aβ42 tend to aggregate (for example, see Jarrell J T et at, The carboxy terminus of the β amyloid protein is critical for the seeding of amyloid formation: implications for the pathogenesis of Alzheimer's disease, Biochemistry, May 11, 1993, 32(18), p. 4693-4697) and constitute the main components of senile plaques (for example, (Glenner G G, et al, Alzheimer's disease: initial report of the purification and characterization of a novel cerebrovascular amyloid protein, Biochemical and Biophysical Research Communications, May 16, 1984, 120(3), p. 885-90. See also Masters C L, et al, Amyloid plaque core protein in Alzheimer disease and Down syndrome, Proceeding National Academy of Science USA, June 1985, 82(12), p. 4245-4249.).

Furthermore, it is known that mutations of APP and presenelin genes, which are observed in familial Alzheimer's disease, increase production of Aβ40 and Aβ42 (for example, see Gouras G K, et al, Intraneuronal Aβ142 accumulation in human brain, American Journal of Pathology, January 2000, 156(1), p. 15-20. Also, see Scheuner D, et al, Nature Medicine, August 1996, 2(8), p. 864-870; and Forman M S, et al, Differential effects of The Swedish mutant amyloid precursor protein on β-amyloid accumulation and secretion in neurons and normeuronal cells, Journal of Biological Chemistry, Dec. 19, 1997, 272(51), p. 32247-32253.). Therefore, compounds which reduce production of Aβ40 and Aβ42 are expected to be agents for controlling progress of Alzheimer's disease or for preventing the disease.

These Aβs are produced when APP is cleaved by beta secretase and subsequently cleaved by gamma secretase. In consideration of this, creation of inhibitors of γ-secretase and β-secretase has been attempted for the purpose of reducing production of Aβs. Many of these known secretase inhibitors are peptides or peptidomimetics such as L-685,458. L-685,458, an aspartyl protease transition state mimic, is a potent inhibitor of γ-secretase activity, (Biochemistry, Aug. 1, 2000, 39(30), p. 8698-8704).

Also of interest in connection with the present invention are: US 2007/0117798 (Eisai, published May 24, 2007); US 2007/0117839 (Eisai, published May 24, 2007); US 2006/0004013 (Eisai, published Jan. 5, 2006); WO 2005/110422 (Boehringer Ingelheim, published Nov. 24, 2005); WO 2006/045554 (Cellzone AG, published May 4, 2006); WO 2004/110350 (Neurogenetics, published Dec. 23, 2004); WO 2004/071431 (Myriad Genetics, published Aug. 26, 2004); US 2005/0042284 (Myriad Genetics, published Feb. 23, 2005) and WO 2006/001877 (Myriad Genetics, published Jan. 5, 2006).

There is a need for new compounds, formulations, treatments and therapies to treat diseases and disorders associated with Aβ. It is, therefore, an object of this invention to provide compounds useful in the treatment or prevention or amelioration of such diseases and disorders.

### SUMMARY OF THE INVENTION

In its many embodiments, the present invention provides a novel class of heterocyclic compounds as gamma secretase modulators (including inhibitors, antagonists and the like), methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with the Aβ using such compounds or pharmaceutical compositions.

One embodiment, of the present invention is directed to compounds of formula (I):

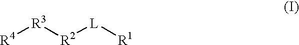

or a pharmaceutically acceptable salt, solvate, ester or prodrug thereof, wherein R¹, R², R³, R⁴, and L are as defined below.

This invention also provides compounds of formula (I).

This invention also provides compounds of formula (I) in pure and isolated form.

This invention also provides compounds of formula (I) selected from the group consisting of: compounds of formulas IA to IE, 1A to 4A, A1.1 to A28.1, A1.2 to A22.2, A24.2 to A28.2, 5.1, 8.1, 11.1, and A1 to A28.

This invention also provides, compounds of formula (I) selected from the group consisting of: compounds of formulas IA to IE.

This invention also provides compounds of formula (I) selected from the group consisting of: compounds of formulas 1A to 4A.

This invention also provides compounds of formula (I) selected from the group consisting of: compounds of formulas A1.1 to A28.1.

This invention also provides compounds of formula (I) selected from the group consisting of: compounds of formulas A1.2 to A22.2, and A24.2 to A28.2.

This invention also provides compounds of formula (I) selected from the group consisting of: compounds of formulas 5.1, 8.1, and 11.1.

This invention also provides compounds of formula (I) selected from the group consisting of: compounds of formulas A1 to A28.

This invention also provides pharmaceutical compositions comprising an effective amount of one or more (e.g., one) compounds of formula (I), or a pharmaceutically acceptable salt, ester or solvate thereof, and a pharmaceutically acceptable carrier.

This invention also provides pharmaceutical compositions comprising an effective amount of one or more (e.g., one) compounds of formula (I), or a pharmaceutically acceptable salt, ester or solvate thereof, and an effective amount of one or more (e.g., one) other pharmaceutically active ingredients (e.g., drugs), and a pharmaceutically acceptable carrier.

The compounds of formula (I) can be useful as gamma secretase modulators and can be useful in the treatment and prevention of diseases such as, for example, central nervous system disorders such as Alzheimers disease and Downs Syndrome.

Thus, this invention also provides methods for: (1) method for modulating (including inhibiting, antagonizing and the like) gamma-secretase; (2) treating one or more neurodegenerative diseases; (3) inhibiting the deposition of amyloid protein (e.g., amyloid beta protein) in, on or around neurological tissue (e.g., the brain); (4) Alzheimer's disease; and (5) treating Downs syndrome; wherein each method comprises administering an effective amount of one or more (e.g., one) compounds of formula (I) to a patient in need of such treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula (I) and the administration of an effective amount of one or more (e.g., one) other pharmaceutical active ingredients (e.g., drugs).

This invention also provides methods for: (1) treating mild cognitive impairment; (2) treating glaucoma; (3) treating cerebral amyloid angiopathy; (4) treating stroke; (5) treating dementia; (6) treating microgliosis; (7) treating brain inflammation; and (8) treating olfactory function loss; wherein each method comprises administering an effective amount of one or more (e.g., one) compounds of formula (I) to a patient in need of such treatment.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described below), the combined quantities of the compound of formula (I) and the other pharmaceutically active ingredient being effective to treat the diseases or conditions mentioned in any of the above methods.

This invention also provides any of the above mentioned methods, pharmaceutical compositions or kit wherein the compound of formula (I) is selected from the group consisting of: compounds of formulas IA to IE, 1A to 4A, A1.1 to A28.1, A1.2 to A22.2, A24.2 to A28.2, 5.1, 8.1, 11.1, and A1 to A28.

This invention also provides any of the above mentioned methods, pharmaceutical compositions or kit wherein the compound of formula (I) is selected from the group consisting of: compounds IA to IE.

This invention also provides any of the above mentioned methods, pharmaceutical compositions or kit wherein the compound of formula (I) is selected from the group consisting of: compounds 1A to 4A.

This invention also provides any of the above mentioned methods, pharmaceutical compositions or kit wherein the compound of formula (I) is selected from the group consisting of; compounds A1.1 to A28.1.

This invention also provides any of the above mentioned methods, pharmaceutical compositions or kit wherein the compound of formula (I) is selected from the group consisting of: compounds A1.2 to A22.2, and A24.2 to A28.2.

This invention also provides any of the above mentioned methods, pharmaceutical compositions or kit wherein the compound of formula (I) is selected from the group consisting of: compounds 5.1, 8.1, and 11.1.

This invention also provides any of the above mentioned methods, pharmaceutical compositions or kit wherein the compound of formula (I) is selected from the group consisting of: compounds A1 to A28.

### DETAILED DESCRIPTION OF THE INVENTION

This invention provides compounds, useful as gamma secretase modulators, of formula (I):

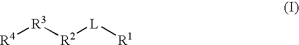

or a pharmaceutically acceptable salt, solvate, ester or prodrug thereof, wherein:

R¹, R², R³, R⁴ and L are each independently selected;

R¹ is selected from the group consisting of: alkyl, alkenyl, alkynyl, cycloalkyl, heterocyclyl (e.g., heterocycloalkyl), cycloalkenyl, aryl (e.g., phenyl), heteroaryl (e.g., pyridyl), heterocyclenyl (i.e., heterocycloalkenyl), fused cycloalkylaryl (i.e., cycloalkyfusedlaryl-), fused heterocycloalkylaryl-(i.e., heterocycloalkylfusedaryl-), fused cycloalkylheteroaryl-(i.e., cycloalkylfusedheteroaryl-), fused heterocycloalkylheteroaryl-(i.e., heterocycloalkylfusedheteroaryl-), fused benzocycloalkylalkyl-(i.e., benzofusedcycloalkylalkyl-), fused benzoheterocycloalkylalkyl-(i.e., benzofusedheterocycloalkylalkyl-), fused heteroarylcycloalkylalkyl-(i.e., heteroarylfusedcycloalkylalkyl-), fused heteroarylheterocycloalkylalkyl-(i.e., heteroarylfusedheterocycloalkylalkyl-), fused cycloalkylarylalkyl-(i.e., cycloalkyfusedlarylalkyl-), fused heterocycloalkylarylalkyl-(i.e. heterocycloalkylfusedarylalkyl-), fused cycloalkylheteroaryialkyl-cycloalkylfusedheteroarylalkyl-), and fused heterocycloalkylheteroarylalkyl-(i.e., heterocycloalkylfusedheteroarylalkyl-), wherein each of said: alkyl, alkenyl, alkynyl, cycloalkyl, heterocyclyl, cycloalkenyl, aryl, heteroaryl, heterocyclenyl fused cycloalkylaryl, fused heterocycloalkylaryl-, fused cycloalkylheteroaryl-, fused heterocycloalkylheteroaryl-, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, and fused heterocycloalkylheteroarylalkyl-R¹ groups is optionally substituted with 1-5 independently selected R²¹ groups;

L is selected from the group consisting of: L is a direct bond, —O—, —N(R⁵)—, —C(R⁶)(R⁷)—, —(C═O)—, —(C═NR^{21A})—, —S—, —S(O)—, and —S(O)₂—;

R² is the fused bicyclic ring:

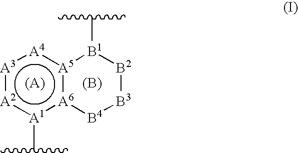

wherein:
(1) Ring (A) is a six membered heteroaryl ring comprising atoms A¹ to A⁶, wherein:
   (a) A¹ is C,
   (b) A⁵ and A⁶ are C,
   (b) A², A³ and A⁴ are each independently selected from the group consisting of: N and C, and wherein each substitutable C is optionally substituted with one R^{21B} group and each R^{21B} for each C is independently selected, and
   (c) provided that at least one (e.g., 1 to 3, or 1 to 2, or 1) of A² to A⁴ is nitrogen, and provided that the total number of nitrogens in Ring (A) is 1 to 3,
(2) Ring (B) (which comprises atoms A⁵, A⁶, and B¹ to B⁴) is a cycloalkyl, cycloalkenyl, heterocycloalkyl, heterocycloalkenyl, heteroaryl or phenyl ring, and
   (a) A⁵ and A⁶ are as defined for Ring (A) above,
   (b) in said phenyl Ring (B):
      (i) B¹ to B⁴ are C, and
      (ii) B², B³, and B⁴ are each optionally substituted with one R^{21B} group (and the substitution on each carbon is independent of the substitutions on the remaining carbons),
   (c) in said cycloalkyl Ring (B):
      (i) B¹ is C,
      (ii) B², B³, and B⁴ are each independently selected from the group consisting of: C, —(C═O)— and —(C═NR^{21A})— (e.g., —(C═N—OR¹⁵)—, and —(C═N—N(R¹⁵)(R¹⁶))—), provided that there are only 0 to 2 moieties selected from the group consisting of —(C═O)— and —(c═NR^{21A})— (i.e. in said cycloalkyl Ring (B) either B², B³, and B⁴ are all C, or one of B², B³, and B⁴ is C and the remaining two are selected from the group consisting of —(C═O)— and —(C═NR^{21A})—, or two of B², B³, and B⁴ are C and the remaining one is selected from the group consisting of: —(C═O)— and —(C═NR^{21A})—), and
      (iii) each substitutable B¹ to B⁴ C is optionally substituted with 1 or 2 independently selected R^{21B} groups (and the substitution on each carbon is independent of the substitutions on the remaining carbons, and those skilled in the art will appreciate that the total number of optional substitutents on a carbon is determined by the number bonds in the ring to the ring atom),
   (d) in said cycloalkenyl Ring (B):
      (i) B¹ is C,
      (ii) B², B³, and B⁴ are each independently selected from the group consisting of: C, —(C═O)— and —(C═NR^{21A})—(e.g., —(C═N—OR¹⁵)—, and —(C═N—N(R¹⁵)(R¹⁶))—), provided that there are only 0 to 2 moieties selected from the group consisting of —(C═O)— and —(C═NR^{21A})—(i.e. in said cycloalkenyl Ring (B) either B², B³, and B⁴ are all C, or one of B², B³, and B⁴ is C and the remaining two are selected from the group consisting of —(C═O)— and —(C═NR^{21A})—, or two of B², B³, and B⁴ are C and the remaining one is selected from the group consisting of: —(C═O)— and —(C═NR^{21A})—),
      (iii) each substitutable B¹ to B⁴ C is optionally substituted with 1 or 2 independently selected R^{21B} groups (and the substitution on each carbon is independent of the substitutions on the remaining carbons, and those skilled in the art will appreciate that the total number of optional substitutents on a carbon is determined by the number bonds in the ring to the ring atom), and
      (iv) said cycloalkenyl Ring (B) comprises one or two double bonds (and in one example one double bond, and in another example two double bonds),
   (e) in said heterocycloalkyl Ring (B):
      (i) B¹ is selected from the group consisting of N and C,
      (ii) B², B³ and B⁴ are each independently selected from the group consisting of: N, C, —(C═O)—, —(C═NR^{21A})— (e.g., —(C═N—OR¹⁵)—, and —(C═N—N(R¹⁵)(R¹⁶))—), O, S, S(O), and S(O)₂, and provided that there are no —O—O— bonds, no —O—S— bonds, no O—S(O) bonds, no —O—S(O)₂ bonds, and no —N—S— bonds in the ring, and provided that the ring does not comprise three adjacent nitrogen atoms,
      (iii) at least one (e.g., 1 to 3, or 1 to 2, or 1) of B¹ to B⁴ is a heteroatom, and provided that when B¹ is a heteroatom said heteroatom is N, and the heteroatoms for B² to B⁴ (when one or more of B² to B⁴ are heteroatoms) are selected from the group consisting of: N, O, S, S(O), and S(O)₂,
      (iv) the total number of heteroatoms in said heterocycloalkyl Ring (B) is 1 to 4, and
      (v) each substitutable B¹ to B⁴ C is optionally substituted with 1 or 2 independently selected R^{21a} groups (and the substitution on each carbon is independent of the substitutions on the remaining carbons, and those skilled in the art will appreciate that the total number of optional substitutents on a carbon is determined by the number bonds in the ring to the ring atom), and
      (vi) each substitutable B² to B⁴ N is optionally substituted with one R^{21A} group and each R^{21A} for each N is independently selected,
   (f) in said heterocycloalkenyl Ring (B):
      (i) B¹ is selected from the group consisting of N and C,
      (ii) B², B³ and B⁴ are each independently selected from the group consisting of: N, C, —(C═O)—, —(C═NR^{21A})—(e.g., —(C═N—OR¹⁵)—, and —(C═N—N(R¹⁵)(R¹⁶))—), O, S, S(O), and S(O)₂, and that there are no —O—O— bonds, no —O—S— bonds, no O—S(O) bonds, no —O—S(O)₂ bonds, and no —N—S— bonds in the ring, and provided that the ring does not comprise three adjacent nitrogen atoms,
      (iii) at least one (e.g., 1 to 4, or 1 to 3, or 1 to 2, or 1) of B¹ to B⁴ is a heteroatom, provided that when B¹ is a heteroatom said heteroatom is N, and the heteroatoms for B² to B⁴ (when one or more of B² to B⁴ are heteroatoms) are selected from the group consisting of: N, O, S, S(O), and S(O)₂,
      (iv) the total number of heteroatoms in said heterocycloalkenyl Ring (B) is 1 to 4, and
      (v) each substitutable B¹ to B⁴ C is optionally substituted with 1 or 2 independently selected R^{21B} groups (and the substitution on each carbon is independent of the substitutions on the remaining carbons, and those skilled in the art will appreciate that the total: number of optional substitutents on a carbon is determined by the number bonds in the ring to the ring atom),
      (vi) each substitutable B² to B⁴ N is optionally substituted with one R^{21A} group and each R^{21A} for each N is independently selected, and
      (vii) said heterocycloalkenyl Ring (B) comprises one or two double bonds (and in one example one double bond, and in another example two double bonds); and
   (g) in said heteroaryl Ring (B):
      (i) B¹ is C,
      (ii) B² to B⁴ are each independently selected from the group consisting of C and N,
      (iii) at least one (e.g., 1 to 4, or 1 to 3, or 1 to 2, or 1) of B² to B⁴ is a heteroatom (e.g., at least one of B² to B⁴ is N), and
      (iv) the total number of heteroatoms in said heteroaryl Ring (B) is 1 to 3 and wherein each substitutable B² to B⁴ C is optionally substituted with one R^{21B} group (and the substitution on each carbon is independent of the substitutions on the remaining carbons);

R³ is selected from the group consisting of: aryl-(e.g., phenyl), heteroaryl-(e.g., pyridyl), cycloalkyl-, cycloalkenyl, cycloalkylalkyl-, heterocyclyl-, heterocyclenyl-, heterocyclylalkyl-, heterocyclyalkenyl-, fused benzacycloalkyl-(i.e., benzofusedcycloalkyl-), fused benzoheterocycloalkyl-(i.e., benzofusedheterocycloalkyl-), fused heteroarylcycloalkyl-(i.e., heteroarylfusedcycloalkyl-), fused heteroarylheterocycloalkyl-(i.e., heteroarylfusedheterocycloalkyl-), fused cycloalkylaryl (i.e., cycloalkyfusedlaryl-), fused heterocycloalkylaryl-(i.e., heterocycloalkylfusedaryl-), fused cycloalkylheteroaryl-(i.e., cycloalkylfusedheteroaryl-), fused hetocycloalkylheteroaryl-(i.e., heterocycloalkylfusedheteroaryl-),

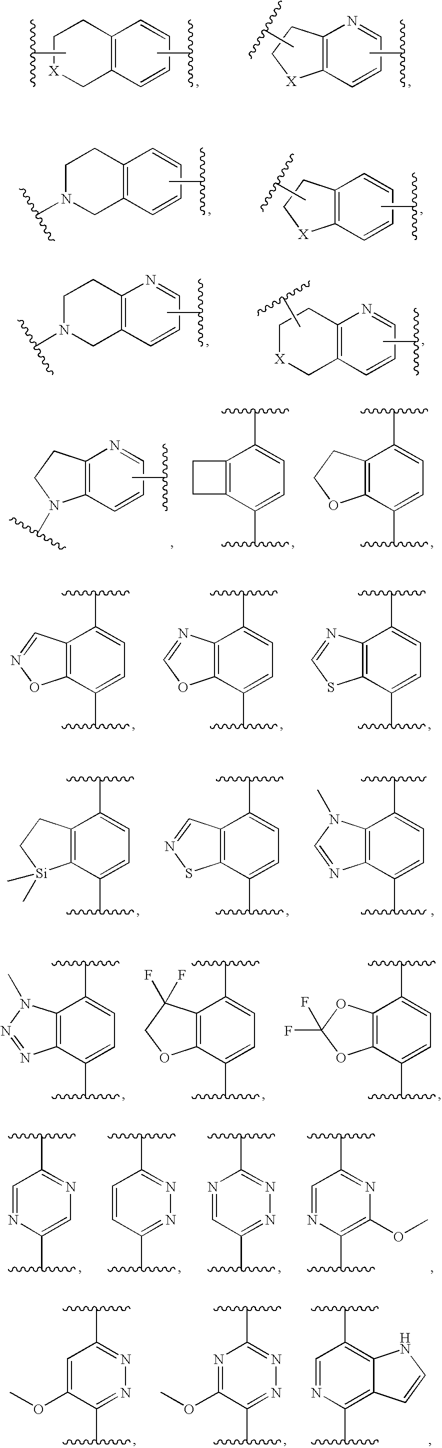

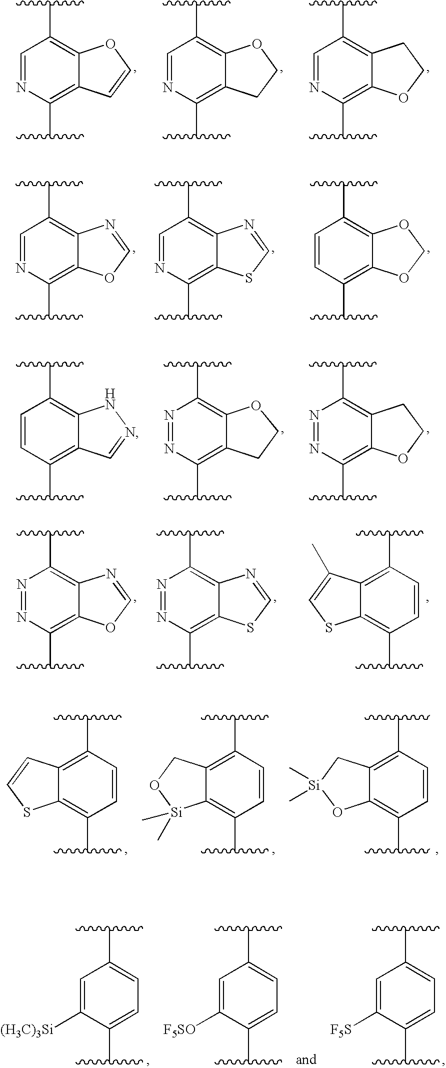

wherein X is selected from the group consisting of: O, —N(R¹⁴)— and —S—; and wherein each of said R³ moieties is optionally substituted with 1-5 independently selected R²¹ groups;

R⁴ is selected from the group consisting of: arylalkoxy-, heteroarylalkoxy-, arylalkylamino-, heteroarylalkylamino-, aryl, heteroaryl, cycloalkyl-, cycloalkenyl, heterocyclyl, heterocyclenyl, and heterocyclyalkyl-, wherein each of said R⁴ arylalkoxy-, heteroarylalkoxy-, arylalkylamino-, heteroarylalkylamino-, aryl, heteroaryl, heterocyclyl, heterocyclenyl, and heterocyclyalkyl- is optionally substituted with 1-5 independently selected R²¹ groups; or

R³ and R⁴ are linked together to form a fused tricyclic ring system wherein R³ and R⁴ are as defined above and the ring linking R³ and R⁴ is an alkyl ring, or a heteroalkyl ring, or an aryl ring, or a heteroaryl ring, or an alkenyl ring, or a heteroalkenyl ring (for example, the tricyclic ring system is formed by linking the atoms adjacent to the atoms by which R³ and R⁴ are bound together);

R⁵ is selected from the group consisting of H, alkyl, alkenyl, alkynyl, cycloalkyl, cycioalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R₁₅)(R¹⁶), —S(O)N(R¹⁵)(R¹⁶), —S(O)₂N(R¹⁵)(R¹⁶), —C(N═NOR¹⁵)R¹⁶, and —P(O)(OR¹⁵)(OR¹⁶); or

R⁶ taken together with R¹ and the nitrogen to which they are bound form a heterocycloalkyl or heterocycloalkenyl ring fused to said R¹ ring, said fused ring is optionally substituted with 1 to 5 independently selected R²¹ groups;

R⁶ and R⁷ are each independently selected from the group consisting of: H, alkyl, alkenyl, alkynyl, aryl, arylalkyl-, alkylaryl-, cycloalkyl, cycloalkylalkyl-, heteroaryl, heteroarylalkyl-, heterocyclyl (i.e., heterocycloalkyl) and heterocyclylalkyl-(i.e., heterocycloalkenyl), wherein independently each of said alkyl, alkenyl and alkynyl, aryl, arylalkyl-, cycloalkyl, cycloalkylalkyl-, heteroaryl, heteroarylalkyl-, heterocyclyl and heterocyclylalkyl- is optionally substituted with 1 to 5 independently selected R²¹ groups; or

R⁶ taken together with R¹ and the carbon to which they are bound form a cycloalkyl, cycloalkenyl, heterocycloalkyl or heterocycloalkenyl ring fused to said R¹ ring, said fused ring is optionally substituted with 1 to 5 independently selected R²¹ groups; or

R⁶ and R⁷ taken together with the carbon to which they are bound form a spirocycloalkyl ring, a spirocycloalkenyl ring, a spiroheterocycloalkyl ring, or a spiroheterocyclalkenyl ring, and wherein the Spiro ring is optionally substituted with 1-5 independently selected R²¹ groups;

R^{15A} and R^{16A} are independently selected from the group consisting of alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)_{q}-alkyl, (R¹⁸)_{q}-cycloalkyl, (R¹⁸)_{q}-cycloalkylalkyl, (R¹⁸)_{q}-heterocyclyl, (R¹⁸)_{q}-heterocyclylalkyl, (R¹⁸)_{q} arylalkyl, (R¹⁸)_{q}-heteroaryl and (R¹⁸)_{q}-heteroarylalkyl, wherein q is 1 to 5 and each R¹⁸ is independently selected (and those skilled in the art will appreciate that the R¹⁸ moieties can be bound to any available substitutable atom);

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)_{q}-alkyl, (R¹⁸)_{q}-cycloalkyl, (R¹⁸)_{q}-cycloalkylalkyl, (R¹⁸)_{q}-heterocyclyl, (R¹⁸)_{q}-heterocyclylalkyl, (R¹⁸)_{q}-aryl, (R¹⁸)_{q}-arylalkyl, (R¹⁸)_{q}-heteroaryl and (R¹⁸)_{q}-heteroarylalkyl, wherein q is 1 to 5 and each R¹⁸ is independently selected (and those skilled in the art will appreciate that the R¹⁸ moieties can be bound to any available substitutable atom); or

each R¹⁸ is independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl); or

alternately, two R¹⁸ moieties on adjacent carbons can be linked together to form:

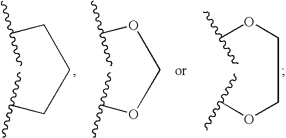

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

each R²¹ group is independently selected from the group consisting of alkyl, alkenyl, alkynyl, cycloalkyl, cycloalkylalkyl, cycloalkenyl, heterocyclyl (i.e., heterocycloalkyl), heterocyclylalkyl (i.e., heterocycloalkylalkyl), aryl, arylalkyl, heteroaryl, heteroarylalkyl, halo, —CN, —OR¹⁵, —C(O)R¹⁵, —C(O)OR¹⁵, —C(O)N(R¹⁵)(R¹⁸), —P(O)(CH₃)₂, —SO(═NR¹⁵)R¹⁶—, —SF₅, —OSF₅, —Si(R^{15A})₃ wherein each R^{15A} is independently selected, —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵, —CH₂N(R¹⁵)(R¹⁶), —N(R¹⁵)S(O)R^{16A}, —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R^{15A}, —N₃, —NO₂, —S(O)₂R^{15A}, —O—N═C(R¹⁵)₂ (wherein each R¹⁵ is independently selected), and —O—N═C(R¹⁵)₂ wherein said R¹⁶ groups are taken together with the carbon atom to which they are bound to form a 5 to 10 membered ring and wherein said ring optionally contains 1 to 3 heteroatoms independently selected from the group consisting of —O—, —S—, —S(O)—, —S(O)₂—, and —NR^{21A};

each R^{21A} is independently selected from the group consisting of H, alkyl, alkenyl, alkynyl, cycloalkyl, cycloalkylalkyl, cycloalkenyl, heterocyclyl (i.e., heterocycloalkyl), heterocyclylalkyl (i.e., heterocycloalkylalkyl), aryl, arylalkyl, heteroaryl, heteroarylalkyl, halo, —OR¹⁵, —CN, -alkyl-(R¹⁵)(R¹⁶), —CH(R¹⁵)(R¹⁶), —CH₂—N(R¹⁵)C(O)R, —CH₂—N(R¹⁵)C(O)N(R¹⁶)(R¹⁷), —CH₂—R¹⁵; —CH₂—N(R¹⁵)(R¹⁶), —C(O)R¹⁵, —C(O)OR¹⁵, —C(O)N(R¹⁵)(R¹⁶), —C(═NOR¹⁵)R¹⁶, —CH₂—N(R¹⁵)S(O)₂R^{16A}, —CH₂—N(R¹⁵)C(O)N(R¹⁶)(R¹⁷), —CH₂—N(R¹⁵)C(O)OR¹⁶, —C(R¹⁵)═NOR¹⁶, —S(O)R^{15A}; —S(O)(OR¹⁵), —S(O)₂(OR¹⁵), —S(O)₂R^{15A}, —S(O)N(R¹⁵)(R¹⁶), —S(O)₂ N(R¹⁵)(R¹⁶), —P(O)(OR¹⁵)(OR¹⁶), —N(R¹⁵)(R¹⁶), —N(R¹⁵)C(O)R¹⁶, —N(R¹⁵)S(O)R^{16A}, —N(R¹⁵)S(O)₂R^{16A}, —N(R¹⁵)S(O)₂ N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —N₃, —NO₂, —P(O)(CH₃)₂, —SO(═NR¹⁵)R¹⁶—, —SF₅, and —OSF₅;

each R^{21B} group is independently selected from the group consisting of H, alkyl, alkenyl, alkynyl, cycloalkyl, cycloalkylalkyl, cycloalkenyl, heterocyclyl (i.e., heterocycloalkyl), heterocyclylalkyl (i.e., heterocycloalkylalkyl), aryl, arylalkyl, heteroaryl, heteroarylalkyl, halo, —OR¹⁵, —CN, -alkyl-(R¹⁵)(R¹⁶), —CH(R¹⁵)(R¹⁶), —CH₂—N(R¹⁵)C(O)R¹⁶, —CH₂—N(R¹⁵)C(O)N(R¹⁶)(R¹⁷), —CH₂—R¹⁵, —CH₂—N(R¹⁵)(R¹⁶), —C(O)R¹⁵, —C(O)OR¹⁵, —C(O)N(R¹⁵)(R¹⁶), —C(═NOR¹⁵)R¹⁶, —CH₂—N(R¹⁵)S(O)₂R^{16A}, —CH₂—N(R¹⁵)C(O)N(R¹⁶)(R¹⁷), —CH₂—N(R¹⁵)C(O)OR¹⁶, —C(R¹⁵)═NOR¹⁶, —SR¹⁵; —S(O)R^{15A}; —S(O)(OR¹⁵), —S(O)(OR¹⁶), —S(O)₂R^{15A}, —S(O)N(R¹⁵)(R¹⁶), —S(O)₂N(R¹⁵)(R¹⁶), —P(O)(OR¹⁵)(OR¹⁶), —N(R¹⁵)(R¹⁶), —N(R¹⁵)C(O)R¹⁶, —N(R¹⁵)S(O)R^{16A}, —N(R¹⁵)S(O)₂R^{16A}, —N(R¹⁵)S(O)₂ N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —N₃, —NO₂, —P(O)(CH₃)₂, —SO(═NR¹⁵)R¹⁶—, —SF₅, —OSF₅, and —Si(R^{15A})₃ wherein each R^{15A} is independently selected;

independently, each alkyl, cycloalkenyl, cycloalkyl, cycloalkylalkyl, heterocyclyl, heterocyclylalkyl, aryl, arylalkyl, heteroaryl, heteroarylalkyl, alkenyl and alkynyl R²¹, R^{21A}, and R^{21B} group is optionally substituted by 1 to 5 independently selected R²² groups wherein each R²² group is independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶, —N(R¹⁵)(R¹⁶), -alkyl-N(R¹⁵)(R¹⁶), —N(R¹⁵)C(O)R¹⁶, —CH₂—N(R¹⁵)C(O)R¹⁶—N(R¹⁵)S(O)R₁₆, —N(R¹⁵)S(O)₂R¹⁶, —CH₂—N(R¹⁵)S(O)₂R¹⁶—N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R^{15A} and —S(O)₂R^{15A}; and

With the proviso that when R³ is aryl and R¹ comprises a 5 or 6-membered aryl or heteroaryl ring, then said 5 or 6-membered aryl or heteroaryl ring is not substituted with an R²¹ group that is selected from the group consisting of the moieties: —O-(5 or 6 membered aryl), —S-(5 or 6 membered aryl), —S(O)₂-(5 or 6 membered aryl), —N(R¹⁵)-(5 or 6 membered aryl), —C(O)-(5 or 6 membered aryl), -alkyl-(5 or 6 membered aryl), —O-(5 or 6 membered heteroaryl), —S-(5 or 6 membered heteroaryl), —S(O)₂-(5 or 6 membered heteroaryl), —N(R¹⁵)-(5 or 6 membered heteroaryl), —C(O)-(5 or 6 membered heteroaryl), and -alkyl-(5 or 6 membered heteroaryl).

Those skilled in the art will appreciate that the above proviso means that when R³ is aryl and R¹ comprises a 5 or 6-membered aryl or heteroaryl ring, then said 5 or 6-membered aryl or heteroaryl ring is not substituted with —O-(5 or 6 membered aryl), —S-(5 or 6 membered aryl), —S(O)₂-(5 or 6 membered aryl), —N(R¹⁵)-(5 or 6 membered aryl), —C(O)-(5 or 6 membered aryl), -alkyl-(5 or 6 membered aryl), —O-(5 or 6 membered heteroaryl), —S-(5 or 6 membered heteroaryl), —S(O)₂-(5 or 6 membered heteroaryl), —N(R¹⁵)-(5 or 6 membered heteroaryl), —C(O)-(5 or 6 membered heteroaryl), or -alkyl-(5 or 6 membered heteroaryl).

In one embodiment R³ is selected from the group consisting of: phenyl and pyridyl, wherein said R³ group is optionally substituted with 1 to 4 independently selected R²¹ groups.

In another embodiment R³ is selected from the group consisting of:

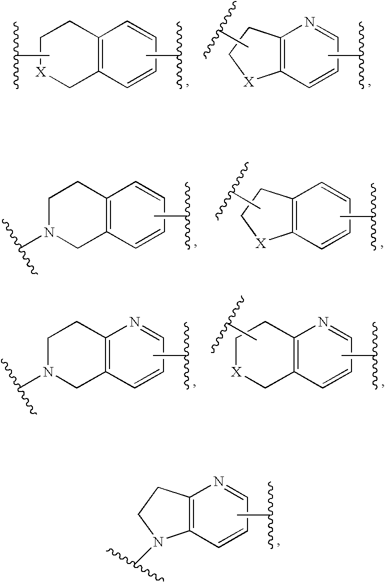

wherein X is selected from the group consisting of: O, —N(R¹⁴)— and —S—; and wherein each of said R³ moieties is optionally substituted with 1-5 independently selected R²¹ groups.

In another embodiment of this invention R³ is selected from the group consisting of: aryl-(e.g., phenyl), heteroaryl-(e.g., pyridyl), cycloalkyl-, cycloalkenyl, cycloalkylalkyl-, heterocyclyl-, heterocyclenyl-, heterocyclylalkyl-, heterocyclyalkenyl-, fused benzocycloalkyl-(i.e., benzofusedcycloalkyl-), fused benzoheterocycloalkyl-(i.e., benzofusedheterocycloalkyl-), fused heteroarylcycloalkyl-(i.e., heteroarylfusedcycloalkyl-), fused heteroarylheterocycloalkyl-(i.e., heteroarylfusedheterocycloalkyl-), fused cycloalkylaryl (i.e., cycloalkyfusedlaryl-), fused heterocycloalkylaryl-(i.e., heterocycloalkylfusedaryl-), fused cycloalkylheteroaryl-(i.e., cycloalkylfusedheteroaryl-), fused heterocycloalkylheteroaryl-(i.e., heterocycloalkylfusedheteroaryl-), and wherein each of said R³ moieties is optionally substituted with 1-5 independently selected R²¹ groups.

In another embodiment of this invention R³ is selected from the group consisting of:

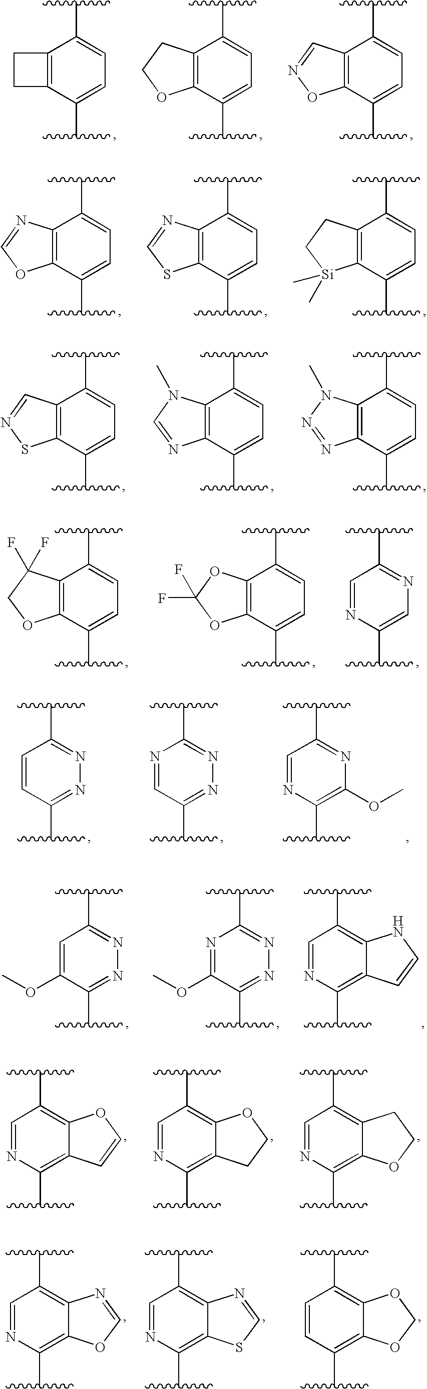

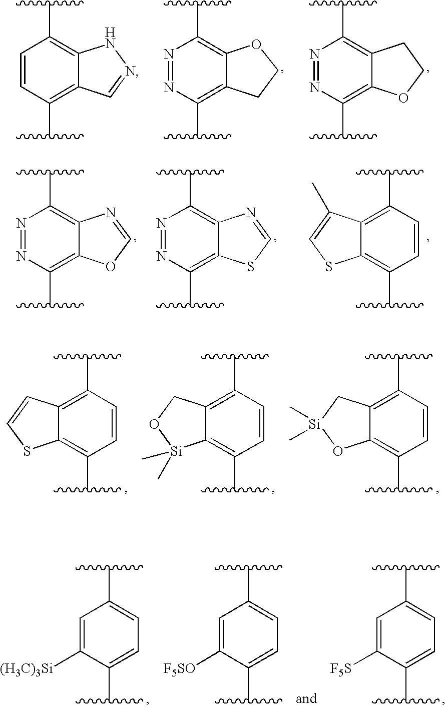

wherein each of said R³ moieties is optionally substituted with 1-5 independently selected R²¹ groups.

In another embodiment R⁴ is a five membered heteroaryl ring optionally substituted with 1 to 4 independently selected R²¹ groups.

Examples of moieties formed when R³ and R⁴ are linked together to form a fused tricyclic ring system include, but are not limited to:

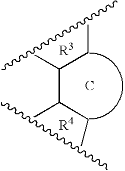

wherein R³ and R⁴ are as defined for formula (I), and Ring C is the ring linking R³ and R⁴, that is Ring C is an alkyl ring, or a heteroalkyl ring, or an aryl ring, or a heteroaryl ring, or an alkenyl ring, or a heteroalkenyl ring.

Examples of moieties formed when R³ and R⁴ are linked together to form a fused tricyclic ring system include, but are not limited to:

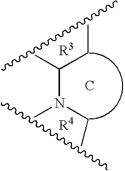

wherein R³ and R⁴ are as defined for formula (I), and Ring C is the ring linking R³ and R⁴, that is Ring C is a heteroalkyl ring, or a heteroaryl ring, or a heteroalkenyl ring.

In one example, the fused tricyclic ring system formed when R³ and R⁴ are linked together is

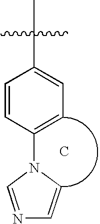

wherein Ring C is a heteroalkyl ring, or a heteroaryl ring, or a heteroalkenyl ring, thus, for example, the tricyclic ring system is formed by linking the atoms adjacent to the atoms by which R³ and R⁴ are bound together), and wherein said fused tricyclic ring system is optionally substituted with 1 to 5 independently selected R²¹ groups.

Other examples of moieties formed when R³ and R⁴ are linked together to form a fused tricyclic ring system include, but are not limited to:

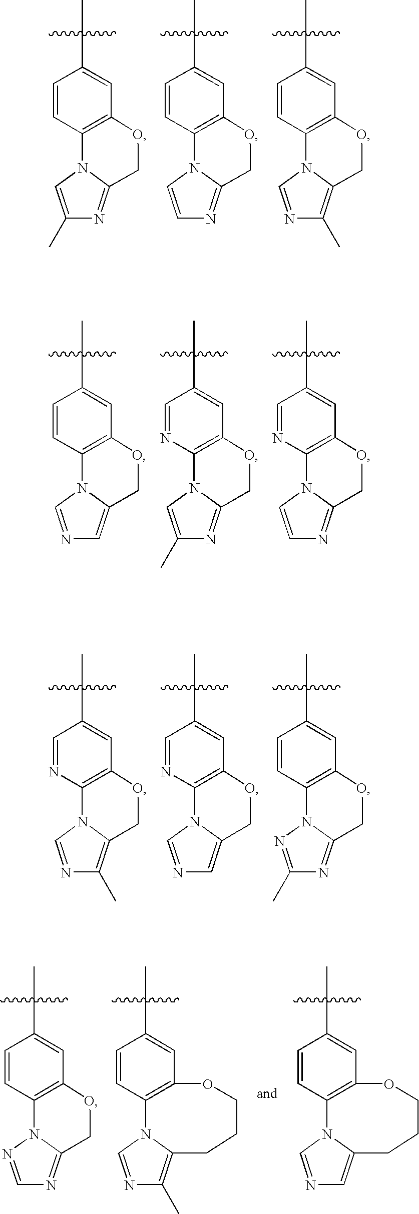

In one embodiment of this invention R³ is bound to A¹ and L is bound to B¹. Thus, in this embodiment the compound of formula (I) is a compound of the formula:

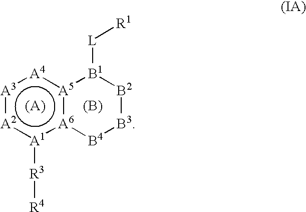

In another embodiment of this invention R³ is bound to B¹ and L is bound to A¹. Thus, in this embodiment the compound of formula (I) is a compound of the formula:

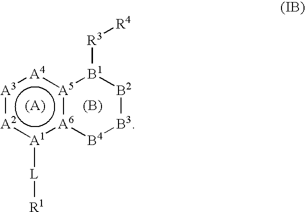

In another embodiment of this invention the R⁴—R³— moiety is:

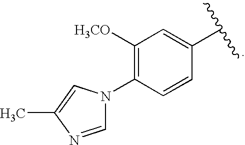

Thus, in another embodiment of this invention the compound of formula (I) is a compound of the formula:

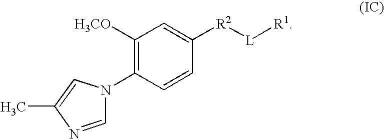

In another embodiment of this invention the compound of formula (I) is a compound of the formula:

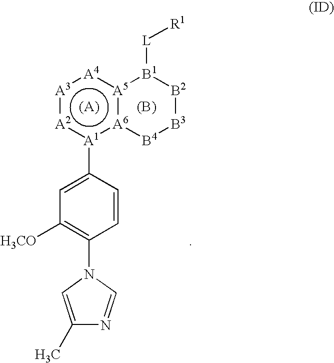

In another embodiment of this invention the compound of formula (I) is a compound the formula:

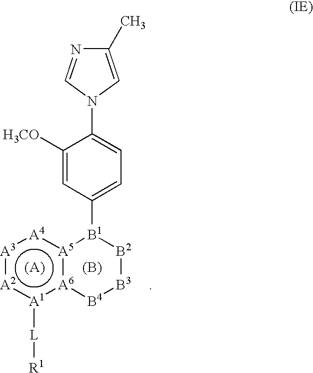

Another embodiment of this is directed to compounds of formula (I) wherein at least one (e.g., 1 to 3, or 1-2, or 1) group selected from the group consisting of: —SF₅, —OSF₅, and —Si(R^{15A})₃ is present, and wherein each R^{15A} is independently selected, and wherein when there is more than one group, each group is independently selected.

Another embodiment of this is directed to compounds of formula (I) wherein at least one (e.g., 1 to 3, or 1-2, or 1) group selected from the group consisting of: —SF₅ and —OSF₅ present, and wherein when there is more than one group, each group is independently selected.

In one embodiment of this invention one group selected from the group consisting of: —SF₅, —OSF₅, and —Si(R^{15A})₃ (wherein each R^{15A} is independently selected) is present in the compounds of formula (I).

In another embodiment of this invention two groups selected from the group consisting of: —SF₅, —OSF₅, and —Si(R^{15A})₃ (wherein each R^{15A} is independently selected) are present in the compounds of formula (I).

In another embodiment of this invention three groups selected from the group consisting of: —SF₅, —OSF₅, and —Si(R^{15A})₃ (wherein each R^{15A} is independently selected) are present in the compounds of formula (I).

In another embodiment of this invention two groups selected from the group consisting of: —SF₅, —OSF₅, and —Si(R^{15A})₃ (wherein each R^{15A} is independently selected) are present in the compounds of formula (I), wherein at least one group is other than —Si(R^{15A})₃.

In another embodiment of this invention three groups selected from the group consisting of: —SF₅, —OSF₅, and —Si(R^{15A})₃ (wherein each R^{15A} is independently selected) are present in the compounds of formula (I), wherein at least one group is other than —Si(R^{15A})₃.

In another embodiment of this invention one group selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) is present in the compounds of formula (I).

In another embodiment of this invention two groups selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) are present in the compounds of formula (I).

In another embodiment of this invention three groups selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) are present in the compounds of formula (I).

In another embodiment of this invention two groups selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) are present in the compounds of formula (I), wherein at least one group is other than —Si(R^{15A})₃.

In another embodiment of this invention three groups selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) are present in the compounds of formula (I), wherein at least one group is other than —Si(R^{15A})₃.

In another embodiment of this invention one group selected from the group consisting of: —SF₅, —OSF₅, and —Si(R^{15A})₃ (wherein each R^{15A} is independently selected from the group consisting of methyl, ethyl and phenyl) is present in the compounds of formula (I).

In another embodiment of this invention two groups selected from the group consisting of: —SF₅, —OSF₅, and —Si(R^{15A})₃ (wherein each R^{15A} is independently selected from the group consisting of methyl, ethyl and phenyl) are present in the compounds of formula (I)

In another embodiment of this invention three groups selected from the group consisting of: —SF₅, —OSF₅, and —Si(R^{15A})₃ (wherein each R^{15A} is independently selected from the group consisting of methyl, ethyl and phenyl) are present in the compounds of formula (I).

In another embodiment of this invention two groups selected from the group consisting of: —SF₅, —OSF₅, and —Si(R^{15A})₃ (wherein each R^{15A} is independently selected from the group consisting of methyl, ethyl and phenyl) are present in the compounds of formula (I), wherein at least one group is other than —Si(R^{15A})₃.

In another embodiment of this invention three groups selected from the group consisting of: —SF₅, —OSF₅, and —Si(R^{15A})₃ (wherein each R^{15A} is independently selected from the group consisting of methyl, ethyl and phenyl) are present in the compounds of formula (I), wherein at least one group is other than —Si(R^{15A})₃.

In another embodiment of this invention one group selected from the group consisting of: —SF₅, —OSF₅, and —Si(R^{15A})₃ (wherein each R^{15A} is independently selected from the group consisting of methyl and ethyl) is present in the compounds of formula (I).

In another embodiment of this invention two groups selected from the group consisting of: —SF₅, —OSF₅, and —Si(R^{15A})₃ (wherein each R^{15A} is independently selected from the group consisting of methyl and ethyl) are present in the compounds of formula (I).

In another embodiment of this invention three groups selected from the group consisting of: —SF₅, —OSF₅, and —Si(R^{15A})₃ (wherein each R^{15A} is independently selected from the group consisting of methyl and ethyl) are present in the compounds of formula (I).

In another embodiment of this invention two groups selected from the group consisting of: —SF₅, —OSF₅, and —Si(R^{15A})₃ (wherein each R^{15A} is independently selected from the group consisting of methyl and ethyl) are present in the compounds of formula (I), wherein at least one group is other than —Si(R^{15A})₃.

In another embodiment of this invention three groups selected from the group consisting of: —SF₅, —OSF₅, and —Si(R^{15A})₃ (wherein each R^{15A} is independently selected from the group consisting of methyl and ethyl) are present in the compounds of formula (I), wherein at least one group is other than —Si(R^{15A})₃.

In another embodiment of this invention one group selected from the group consisting of: —SF₅, —OSF₅, and —Si(R^{15A})₃ is present in the compounds of formula (I), and said —Si(R^{15A})₃ group is selected from the group consisting of: —Si(CH₃)₃, —Si(CH₃)₂ phenyl, and —Si(CH₂ CH₃)₂CH₃.

In another embodiment of this invention two groups selected from the group consisting of: —SF₅, —OSF₅, and —Si(R^{15A})₃ are present in the compounds of formula (I), and said —Si(R^{15A})₃ group is selected from the group consisting of: —Si(CH₃)₃, —Si(CH₃)₂phenyl, and —Si(CH₂ CH₃)₂CH₃.

In another embodiment of this invention three groups selected from the group consisting of: —SF₅, —OSF₅, and —Si(R^{15A})₃ are present in the compounds of formula (I), and said —Si(R^{15A})₃ group is selected from the group consisting of: —Si(CH₃)₃, —Si(CH₃)₂phenyl, and —Si(CH₂ CH₃)₂CH₃.

In another embodiment of this invention two groups selected from the group consisting of: —SF₅, —OSF₅, and —Si(R^{15A})₃ are present in the compounds of formula (I), wherein at least one group is other than —Si(R^{15A})₃, and said —Si(R^{15A})₃ group is selected from the group consisting of: —Si(CH₃)₃, —Si(CH₃)₂phenyl, and —Si(CH₂ CH₃)₂CH₃.

In another embodiment of this invention three groups selected from the group consisting of: —SF₅, —OSF₅, and —Si(R^{15A})₃ are present in the compounds of formula (I), wherein at least one group is other than —Si(R^{15A})₃, and said —Si(R^{15A})₃ group is selected from the group consisting of: —Si(CH₃)₃, —Si(CH₃)₂phenyl, and —Si(CH₂ CH₃)₂CH₃.

In another embodiment of this invention one group selected from the group consisting of: —SF₅, —OSF₅, and —Si(R^{15A})₃ is present in the compounds of formula (I), and said —Si(R^{15A})₃ group is selected from the group consisting of: —Si(CH₃)₃ and —Si(CH₂ CH₃)₂CH₃.

In another embodiment of this invention two groups selected from the group consisting of: —SF₅, —OSF₅, and —Si(R^{15A})₃ are present in the compounds of formula (I), and said —Si(R^{15A})₃ group is selected from the group consisting of: —Si(CH₃)₃ and —Si(CH₂ CH₃)₂CH₃.

In another embodiment of this invention three groups selected from the group consisting of: —SF₅, —OSF₅, and —Si(R^{15A})₃ are present in the compounds of formula (I), and said —Si(R^{15A})₃ group is selected from the group consisting of: —Si(CH₃)₃ and —Si(CH₂ CH₃)₂CH₃.

In another embodiment of this invention two groups selected from the group consisting of: —SF₅, —OSF₅, and —Si(R^{15A})₃ are present in the compounds of formula (I), wherein at least one group is other than —Si(R^{15A})₃, and said —Si(R^{15A})₃ group is selected from the group consisting of: —Si(CH₃)₃ and —Si(CH₂ CH₃)₂CH₃.

In another embodiment of this invention three groups selected from the group consisting of: —SF₅, —OSF₅, and —Si(R^{15A})₃ are present in the compounds of formula (I), wherein at least one group is other than —Si(R^{15A})₃, and said —Si(R^{15A})₃ group is selected from the group consisting of: —Si(CH₃)₃, —Si(CH₃)₂phenyl, and —Si(CH₂ CH₃)₂CH₃.

In another embodiment of this invention one group selected from the group consisting of: —SF₅, —OSF₅, and —Si(CH₃)₃ is present.

In another embodiment of this invention two groups selected from the group consisting of: —SF₅, —OSF₅, and —Si(CH₃)₃ are present in the compounds of formula (I).

In another embodiment of this invention three groups selected from the group consisting of: —SF₅, —OSF₅, and —Si(CH₃)₃ are present in the compounds of formula (I).

In another embodiment of this invention two groups selected from the group consisting of: —SF₅, —OSF₅, and —Si(CH₃)₃ are present in the compounds of formula (I), wherein at least one group is other than —Si(CH₃)₃.

In another embodiment of this invention three groups selected from the group consisting of: —SF₅, —OSF₅, and —Si(R²⁴)₃ are present in the compounds of formula (I), wherein at least one group is other than —Si(CH₃)₃.

In another embodiment of this invention one group selected from the group consisting of: —SF₅ and —OSF₅ is present in the compounds of formula (I),

In another embodiment of this invention two groups selected from the group consisting of: —SF₅ and —OSF₅ are present in the compounds of formula (I).

In another embodiment of this invention three groups selected from the group consisting of: —SF₅ and —OSF₅ are present in the compounds of formula (I)

In another embodiment of this invention one —SF₅ group is present in the compounds of formula (I).

In another embodiment of this invention two —SF₅ groups are present in the compounds of formula (I).

In another embodiment of this invention three —SF₅ groups are present in the compounds of formula (I).

In another embodiment of this invention one —OSF₅ group is present in the compounds of formula (I).

In another embodiment of this invention two —OSF₅ groups are present in the compounds of formula (I).

In another embodiment of this invention three —OSF₅ groups are present in the compounds of formula (I).

In another embodiment of this invention one —Si(R^{15A})₃ (wherein each R^{15A} is independently selected) group is present in the compounds of formula (I).

In another embodiment of this invention two —Si(R^{15A})₃ (wherein each R^{15A} is independently selected) groups are present in the compounds of formula (I).

In another embodiment of this invention three —Si(R^{15A})₃ (wherein each R^{15A} is independently selected) groups are present in the compounds of formula (I).

In another embodiment of this invention one —Si(R^{15A})₃ (wherein each R^{15A} is independently selected from the group consisting of alkyl (e.g., methyl and ethyl) and aryl (e.g., phenyl)) is present in the compounds of formula (I).

In another embodiment of this invention two —Si(R^{15A})₃ (wherein each R^{15A} is independently selected from the group consisting of alkyl (e.g., methyl and ethyl) and aryl (e.g., phenyl)) is present in the compounds of formula (I).

In another embodiment of this invention three —Si(R^{15A})₃ (wherein each R^{15A} is independently selected from the group consisting of alkyl (e.g., methyl and ethyl) and aryl (e.g., phenyl)) is present in the compounds of formula (I).

In another embodiment of this invention one —Si(R^{15A})₃ (wherein each R^{15A} is independently selected from the group consisting of methyl, ethyl and phenyl) is present in the compounds of formula (I).

In another embodiment of this invention two —Si(R^{15A})₃ (wherein each R^{15A} is independently selected from the group consisting of methyl, ethyl and phenyl) is present in the compounds of formula (I).

In another embodiment of this invention three —Si(R^{15A})₃ (wherein each R^{15A} is independently selected from the group consisting of methyl, ethyl and phenyl) is present in the compounds of formula (I).

In another embodiment of this invention one —Si(R^{15A})₃ (wherein each R^{15A} is independently selected from the group consisting of methyl and ethyl) is present in the compounds of formula (I).

In another embodiment of this invention two —Si(R^{15A})₃ (wherein each R^{15A} is independently selected from the group consisting of methyl and ethyl) is present in the compounds of formula (I).

In another embodiment of this invention three —Si(R^{15A})₃ (wherein each R^{15A} is independently selected from the group consisting of methyl and ethyl) is present in the compounds of formula (I).

In another embodiment of this invention one —Si(R^{15A})³ group is present in the compounds of formula (I), and said —Si(R^{15A})₃ group is selected from the group consisting of: —Si(CH₃)₃, —Si(CH₃)₂phenyl, and —Si(CH₂ CH₃)₂CH₃.

In another embodiment of this invention two —Si(R^{15A})₃ groups are present in the compounds of formula (I), and said —Si(R^{15A})³ groups are independently selected from the group consisting of: —Si(CH₃)₃, —Si(CH₃)₂phenyl, and —Si(CH₂ CH₃)₂CH₃.

In another embodiment of this invention three —Si(R^{15A})₃ groups are present in the compounds of formula (I), and said —Si(R^{15A})₃ groups are independently selected from the group consisting of: —Si(CH₃)₃, —Si(CH₃)₂phenyl, and —Si(CH₂ CH₃)₂CH₃.

In another embodiment of this invention one —Si(R^{15A})³ group is present in the compounds of formula (I), and said —Si(R^{15A})³ group is selected from the group consisting of: —Si(CH₃)₃ and —Si(CH₂ CH₃)₂CH₃.

In another embodiment of this invention two —Si(R^{15A})³ groups are present in the compounds of formula (I), and said —Si(R^{15A})³ groups are independently selected from the group consisting of: —Si(CH₃)₃ and —Si(CH₂ CH₃)₂CH₃.

In another embodiment of this invention three —Si(R^{15A})₃ groups are present in the compounds of formula (I), and said —Si(R^{15A})₃ groups are independently selected from the group consisting of: —Si(CH₃)₃ and —Si(CH₂ CH₃)₂CH₃.

In another embodiment of this invention one —Si(R^{15A})³ group is present in the compounds of formula (I), and said —Si(R^{15A})³ group is —Si(CH₃)₃,

In another embodiment of this invention two —Si(R^{15A})³ groups are present in the compounds of formula (I), and said —Si(R^{15A})₃ groups are —Si(CH₃)₃.

In another embodiment of this invention three —Si(R^{15A})₃ groups are present in the compounds of formula (I), and said —Si(R^{15A})³ groups are —Si(CH₃)₃.

In another embodiment of this invention one group selected from the group consisting of: —SF₅, —OSF₅, —Si(CH₃)₃, —Si(CH₃)₂phenyl, and —Si(CH₂ CH₃)₂CH₃) is present in the compounds of formula (I).

In another embodiment of this invention one group selected from the group consisting of: —SF₅, —OSF₅, —Si(CH₃)₃, and —Si(CH₂ CH)CH₃) is present in the compounds of formula (I).

In another embodiment of this invention one group selected from the group consisting of: —SF₅, —OSF₅, and —Si(CH₃)₃, is present in the compounds of formula (I).

In another embodiment of this invention one —SF₅ group is present in the compounds of formula (I), and one or two additional groups selected from the group consisting of: —SF₅, —OSF₅, and —Si(R^{15A})₃ (wherein each R^{15A} is independently selected) are also present in the compounds of formula (I).

In another embodiment of this invention one —SF₅ group is present in the compounds of formula (I), and one or two additional groups selected from the group consisting of: —OSF₅, and —Si(R^{15A})₃ (wherein each R^{15A} is independently selected) are also present in the compounds of formula (I).

In another embodiment of this invention one —OSF₅ group is present in the compounds of formula (I), and one or two additional groups selected from the group consisting of: —SF₅, —OSF₅, and —Si(R^{15A})₃ (wherein each R^{15A} is independently selected) are also present in the compounds of formula (I).

In another embodiment of this invention one —OSF₅ group is present in the compounds of formula (I), and one or two additional groups selected from the group consisting of: —SF₅ and —Si(R^{15A})₃ (wherein each R^{15A} is independently selected) are also present in the compounds of formula (I).

In another embodiment of this invention one —SF₅ group is present in the compounds of formula (I), and one or two additional groups selected from the group consisting of: —SF₅ and —OSF₅ are also present in the compounds of formula (I).

In another embodiment of this invention one —OSF₅ group is present in the compounds of formula (I), and one or two additional groups selected from the group consisting of: —SF₅ and —OSF₅ are also present in the compounds of formula (I).

In another embodiment of this invention one —Si(R^{15A})₃ (wherein each R^{15A} is independently selected) group is present in the compounds of formula (I), and one or two groups selected from the group consisting of: —SF₅, —OSF₅, and —Si(R^{15A})₃ (wherein each R^{15A} is independently selected) are also present in the compounds of formula (I).

In another embodiment of this invention one —Si(R^{15A})₃ (wherein each R^{15A} is independently selected) group is present in the compounds of formula (I), and one or two groups selected from the group consisting of: —SF₅ and —OSF₅ are also present in the compounds of formula (I).

In another embodiment of this invention at least one group selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) is present in the compounds of formula (I).

In another embodiment of this invention at least one group selected from the group consisting of: —SF₅, —OSF₅, and —Si(R^{15A})₃ (wherein each R^{15A} is independently selected from the group consisting of alkyl (e.g., methyl and ethyl) and phenyl) is present in the compounds of formula (I).

In another embodiment of this invention at least one group selected from the group consisting of: —SF₅, —OSF₅, and —Si(R^{15A})₃ (wherein each R^{15A} is independently selected from the group consisting of methyl, ethyl and phenyl) is present in the compounds of formula (I).

In another embodiment of this invention at least one group selected from the group consisting of: —SF₅, —OSF₅, —Si(CH₃)₃, —Si(CH—)₂ phenyl, and —Si(CH₂ CH₃)₂CH₃) is present in the compounds of formula (I).

In another embodiment of this invention at least one group selected from the group consisting of: —SF₅, —OSF₅, and —Si(CH₃)₃ is present in the compounds of formula (I).

In another embodiment of this invention one group selected from the group consisting of: —SF₅, —OSF₅, and —Si(R^{15A})₃ (wherein each R^{15A} is independently selected) is present in the compounds of formula (I).

In another embodiment of this invention one group selected from the group consisting of: —SF_{S}, —OSF₅, and —Si(R^{15A})₃ (wherein each R^{15A} is independently selected from the group consisting of alkyl (e.g., methyl and ethyl) and aryl (e.g., phenyl)) is present in the compounds of formula (I).

In another embodiment of this invention one group selected from the group consisting of: —SF₅, —OSF₅, and —Si(R^{15A})₃ (wherein each R^{15A} is independently selected from the group consisting of alkyl (e.g., methyl and ethyl) and phenyl) is present in the compounds of formula (I).

In another embodiment of this invention one group selected from the group consisting of: —SF₅, —OSF₅, and —Si(R^{15A})₃ (wherein each R^{15A} is independently selected from the group consisting of methyl, ethyl and phenyl) is present in the compounds of formula (I).

In another embodiment of this invention one group selected from the group consisting of: —SF₅, —OSF₅, —Si(CH₃)₃, —Si(CH₃)₂phenyl, and —Si(CH₂ CH₃)₂CH₃) is present in the compounds of formula (I).

In another embodiment of this invention one group selected from the group consisting of: —SF₅, —OSF₅, —Si(CH₃)₃, and —Si(CH₂ CH₃)₂CH₃) is present in the compounds of formula (I).

In another embodiment of this invention one group selected from the group consisting of: —SF₅, —OSF₅, and —Si(CH₃), is present in the compounds of formula (I).

In another embodiment of this invention two groups selected from the group consisting of: —SF₅, —OSF₅, and —Si(R^{15A})₃ (wherein each R^{15A} is independently selected) are present in the compounds of formula (I).

In another embodiment of this invention two groups independently selected from the group consisting of: —SF₅, —OSF₅, and —Si(R^{15A)}₃ (wherein each R^{15A} is independently selected from the group consisting of alkyl (e.g., methyl and ethyl) and aryl (e.g., phenyl)) are present in the compounds of formula (I).

In another embodiment of this invention two groups independently selected from the group consisting of: —SF₅, —OSF₅, and —Si(R^{15A})₃ (wherein each R^{15A} is independently selected from the group consisting of alkyl (e.g., methyl and ethyl) and phenyl) are present in the compounds of formula (I).

In another embodiment of this invention two groups selected from the group consisting of: —SF₅, —OSF₅, and —Si(R^{15A})₃ (wherein each R^{15A} is independently selected from the group consisting of methyl, ethyl and phenyl) are present in the compounds of formula (I).

In another embodiment of this invention two groups independently selected from the group consisting of: —SF₅, —OSF₅, —Si(CH₃)ₐ₃, —Si(CH₃)₂phenyl, and —Si(CH₂ CH₃)₂CH₃) is present in the compounds of formula (I).

In another embodiment of this invention two groups independently selected from the group consisting of: —SF₅, —OSF₅, —Si(CH₃)₃, and —Si(CH₂ CH₃)₂CH₃) are present in the compounds of formula (I).

In another embodiment of this invention two groups independently selected from the group consisting of: —SF₅, —OSF₅, and —Si(CH₃)₃ are present in the compounds of formula (I).

In another embodiment of this invention three groups selected from the group consisting of: —SF₅, —OSF₅, and —Si(R^{15A})₃ (wherein each R^{15A} is independently selected) are present in the compounds of formula (I)I.

In another embodiment of this invention three groups independently selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) are present in the compounds of formula (I).

In another embodiment of this invention three groups independently selected from the group consisting of: —SF₅, —OSF₅, and —Si(R^{15A})₃ (wherein each R^{15A} is independently selected from the group consisting of alkyl (e.g., methyl and ethyl) and phenyl) are present in the compounds of formula (I).

In another embodiment of this invention three groups selected from the group consisting of: —SF₅, —OSF₅, and —Si(R^{15A})₃ (wherein each R^{15A} is independently selected from the group consisting of methyl, ethyl and phenyl) are present in the compounds of formula (I).

In another embodiment of this invention three groups independently selected from the group consisting of: —SF₅, —OSF₅, —Si(CH₃)₃, —Si(CH₃)₂phenyl, and —Si(CH₂ CH₃)₂CH₃) is present in the compounds of formula (I).

In another embodiment of this invention three groups independently selected from the group consisting of: —SF₅, —OSF₅, —Si(CH₃)₃, and —Si(CH₂ CH₃)₂CH₃) are present in the compounds of formula (I).

In another embodiment of this invention three groups independently selected from the group consisting of: —SF, —OSF₅, and —Si(CH₃)₃ are present in the compounds of formula (I).

In another embodiment of this invention at least one group selected from the group consisting of: —SF₅, —OSF₅, and —Si(R^{15A}) (wherein each R^{15A} is the same or different alkyl group) is present in the compounds of formula (I).

In another embodiment of this invention at least one group selected from the group consisting of: —SF₅, —OSF₅, and —Si(R^{15A})₃ (wherein each R^{15A} is independently selected from the group consisting of methyl and ethyl) is present in the compounds of formula (I).

In another embodiment of this invention one —SF₅ group is present in the compounds of formula (I), and one or two groups selected from the group consisting of: —SF₅ and —OSF₅ are also present in the compounds of formula (I).

In another embodiment of this invention one —OSF₅ group is present in the compounds of formula (I), and one or two groups selected from the group consisting of: —SF₅ and —OSF₅ are also present in the compounds of formula (I).

In another embodiment of this invention L is —C(R⁶)(R⁷)—.

In another embodiment of this invention L is —C(R⁶)(R⁷)— wherein R⁶ and R⁷ are taken together with the carbon atom to which they are bound to form a spirocycloalkyl ring (e.g., cyclopropyl).

In another embodiment of this invention L is —C(R⁶)(R⁷)— wherein R⁶ and R⁷ are taken together with the carbon atom to which they are bound to form a spirocycloalkenyl ring.

In another embodiment of this invention L is —C(R⁶)(R⁷)— wherein R⁶ and R⁷ are taken together with the carbon atom to which they are bound to form a spiroheterocycloalkyl ring.

In another embodiment of this invention L is —C(R⁶)(R⁷)— wherein R⁶ and R⁷ are taken together with the carbon atom to which they are bound to form a spiroheterocycloalkenyl ring.

In another embodiment of this invention L is —C(R⁶)(R⁷)— wherein R⁶ and R⁷ are independently selected from the group consisting of: H, alkyl, and alkyl substituted with one R²¹ group.

In another embodiment of this invention L is —C(R⁶)(R⁷)— wherein R⁶ and R⁷ are independently selected from the group consisting of: H, methyl, and methyl substituted with one R²¹ group.

In another embodiment of this invention L is —C(R⁶)(R⁷)— wherein R⁶ and R⁷ are independently selected from the group consisting of: H, alkyl, and alkyl substituted with one R²¹ group wherein said R²¹ group is —OR¹⁵.

In another embodiment of this invention L is —C(R⁶)(R⁷)— wherein R⁶ and R⁷ are independently selected from the group consisting of: H, alkyl, and alkyl substituted with one R²¹ group wherein said R²¹ group is —OR¹⁵, and said R¹⁵ is H (i.e., said R²¹ group is —OH).

In another embodiment of this invention L is selected from the group consisting of:

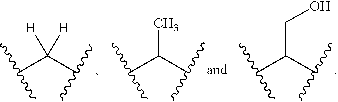

In another embodiment of this invention L is —CH₂—.

In another embodiment of this invention L is —CH(CH₃)—,

In another embodiment of this invention L is —CH(CH₂OH)—.

In another embodiment of this invention, R¹ is selected from the group consisting of: alkyl, alkenyl, alkynyl, cycloalkyl, heterocyclyl (e.g., heterocycloalkyl), cycloalkenyl, aryl (e.g., phenyl), heteroaryl (e.g., pyridyl), heterocyclenyl (i.e., heterocycloalkenyl), wherein each of said: alkyl, alkenyl, alkynyl, cycloalkyl, heterocyclyl, cycloalkenyl, aryl, heteroaryl, and heterocyclenyl R¹ groups is optionally substituted with 1-5 independently selected R²¹ groups.

In another embodiment of this invention R¹ is selected from the group consisting of: fused cycloalkylaryl (i.e., cycloalkyfusediaryl-), fused heterocycloalkylaryl-(i.e., heterocycloalkylfusedaryl-), fused cycloalkylheteroaryl-(i.e., cycloalkylfusedheteroaryl-), fused heterocycloalkylheteroaryl-(i.e., heterocycloalkylfusedheteroaryl-), fused benzocycloalkylalkyl-(i.e., benzofusedcycloalkylalkyl-), fused benzoheterocycloalkylalkyl-(i.e., benzofusedheterocycloalkylalkyl-), fused heteroaryicycloalkylalkyl-(i.e., heteroarylfusedcycloalkylalkyl-), fused heteroarylheterocycloalkylalkyl-(i.e., heteroarylfusedheterocycloalkylalkyl-), fused cycloalkylarylalkyl-(i.e., cycloalkyfusedlarylalkyl-), fused heterocycloalkylarylalkyl-(i.e., heterocycioalkylfusedarylalkyl-), fused cycloalkylheteroarylalkyl-(i.e., cycloalkylfusedheteroarylalkyl-), and fused heterocycloalkylheteroarylalkyl-(i.e., heterocycloalkylfusedheteroarylalkyl-), wherein each of said R¹ groups is optionally substituted with 1-5 independently selected R²¹ groups

In another embodiment of this invention R¹ is phenyl.

In another embodiment of this invention R¹ is phenyl substituted with 1 to 3 halo atoms.

In another embodiment of this invention R¹ is phenyl substituted with 1 to 3 F atoms.

In another embodiment of this invention R¹ is selected from the group consisting of:

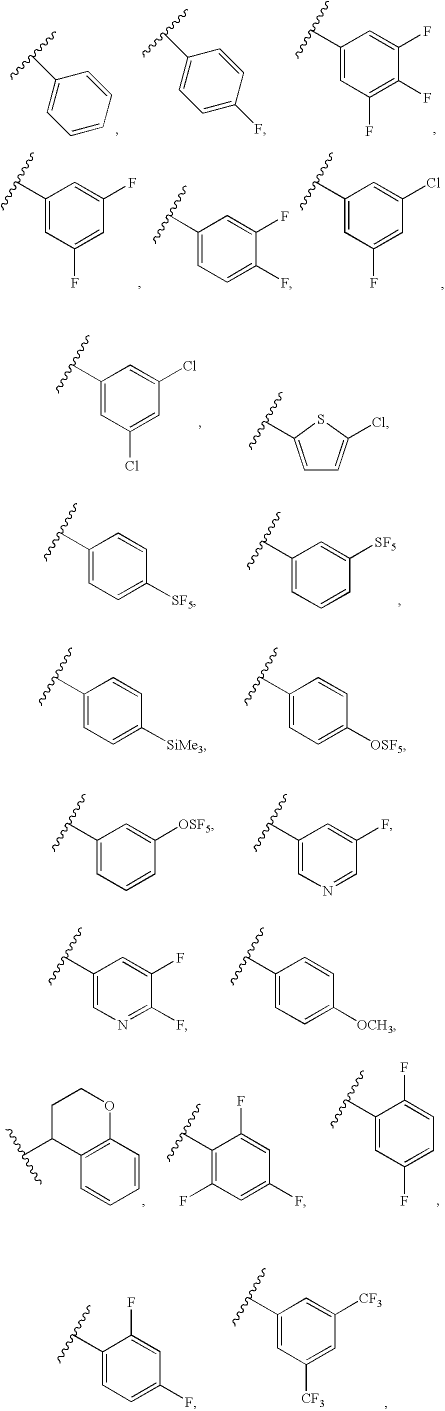

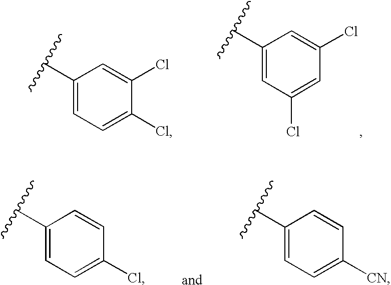

In another embodiment of this invention R¹ is selected from the group consisting of:

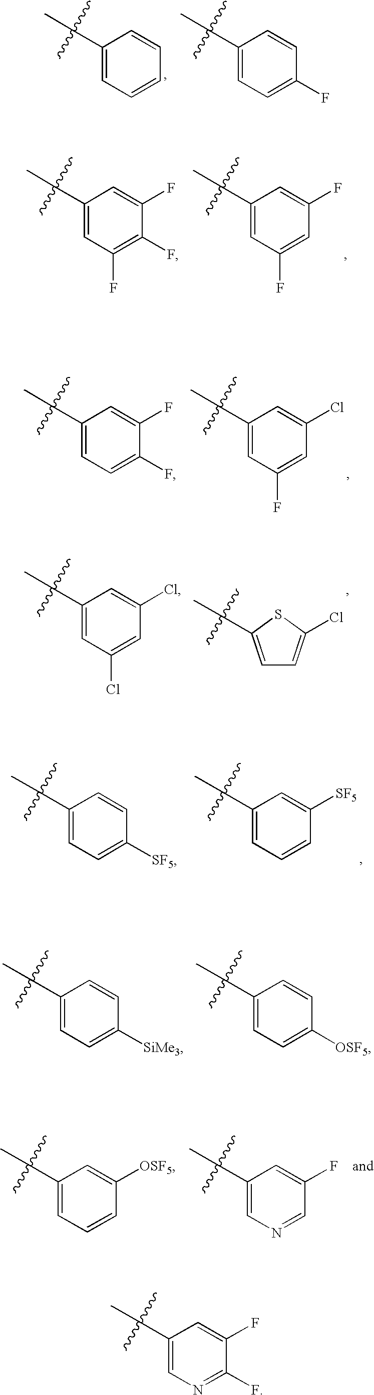

In another embodiment of this invention R¹ is selected from the group consisting of:

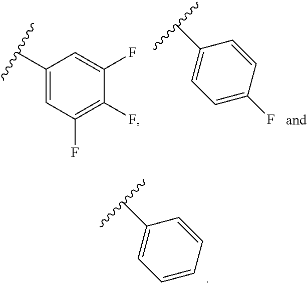

In another embodiment of this invention R¹ is selected from the group consisting of:

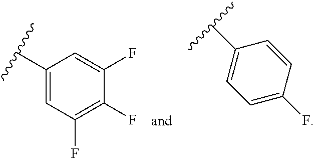

In another embodiment of this invention R¹ is phenyl.

In another embodiment of this invention R¹ is:

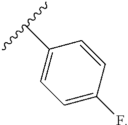

In another embodiment of this invention R¹ is:

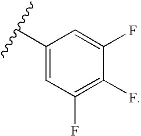

In another embodiment of this invention R¹ is:

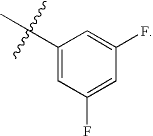

In another embodiment of this invention R¹ is:

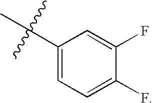

In another embodiment of this invention R¹ is:

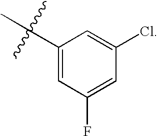

In another embodiment of this invention R¹ is:

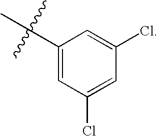

In another embodiment of this invention R¹ is:

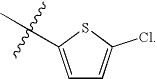

In another embodiment of this invention R¹ is:

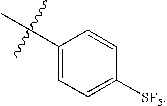

In another embodiment of this invention R¹ is:

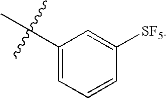

In another embodiment of this invention R¹ is:

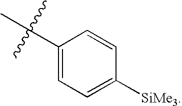

In another embodiment of this invention R¹ is:

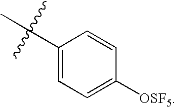

In another embodiment of this invention R¹ is:

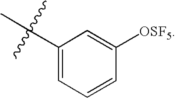

In another embodiment of this invention R¹ is:

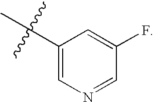

In another embodiment of this invention R¹ is:

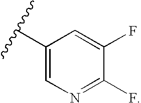

In another embodiment of this invention R¹ is:

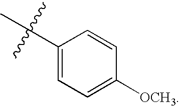

In another embodiment of this invention R¹ is:

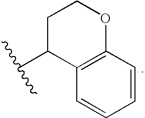

In another embodiment of this invention R¹ is:

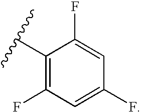

In another embodiment of this invention R¹ is:

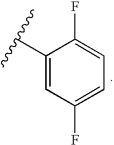

In another embodiment of this invention R¹ is:

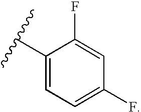

In another embodiment of this invention R¹ is:

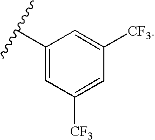

In another embodiment of this invention R¹ is:

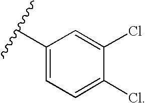

In another embodiment of this invention R¹ is:

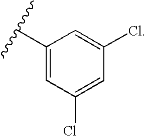

In another embodiment of this invention R¹ is:

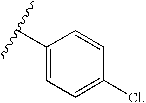

In another embodiment of this invention R¹ is:

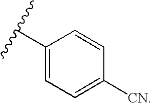

In another embodiment, R¹ is phenyl substituted with 1-3 halos independently selected from the group consisting of F and Cl. In one example said phenyl is substituted with one F and one Cl.

In another embodiment R¹ is aryl (e.g., phenyl) substituted with 1 to 3 independently selected R²¹ moieties wherein at least one R²¹ moiety is selected from the group consisting of —SF₅, —OSF₅ and —Si(R^{15A})₃ (and in one example each R^{15A} is the same or different alkyl, and in another example the —Si(R²⁴)³ group is —Si(CH₃)₃ or —Si(CH₂ CH₃)₂CH₃, and in another example the —Si(R²⁴)³ group is —Si(CH₃)₃).

In another embodiment R¹ is aryl (e.g., phenyl) substituted with 1 to 3 independently selected R²¹ moieties wherein at least one R²¹ moiety is selected from the group consisting of —SF₅ and —OSF₅.

In another embodiment R¹ is aryl (e.g., phenyl) substituted with 1 to 3 R²¹ moieties independently selected from the group consisting of: halo (e.g., F), —SF₅, —OSF₅ and —Si(R^{15A})₃ (and in one example each R^{15A} is the same or different alkyl, and in another example the —Si(R^{15A})³ group is —Si(CH₃)₃ or —Si(CH₂ CH₃)₂CH₃, and in another example the —Si(R^{15A})₃ group is —Si(CH₃)₃), and wherein at least one R²¹ moiety is selected from the group consisting of —SF₅, —OSF₅ and —Si(R^{15A})₃ (and in one example each R^{15A} is the same or different alkyl, and in another example the —Si(R^{15A})₃ group is —Si(CH₃)₃ or —Si(CH₂ CH₃)₂CH₃, and in another example the —Si(R²⁴)₃ group is —Si(CH₃)₃).

In another embodiment R¹ is aryl (e.g., phenyl) substituted with 1 to 3 R²¹ moieties independently selected from the group consisting of: halo (e.g., F), —SF₅ and —OSF₅, and wherein at least one R² moiety is selected from the group consisting of —SF₅ and —OSF₅.

In another embodiment R¹ is aryl (e.g., phenyl) substituted with 1 to 3 independently selected R²¹ moieties wherein at least one R²¹ moiety is selected from the group consisting of —SF₅, —OSF₅ and —Si(R^{15A})₃ (and in one example each R^{15A} is the same or different alkyl, and in another example the —Si(R^{15A})₃ group is —Si(CH₃)₃or —Si(CH₂CH₃)₂ CH₃ and in another example the —Si(R^{15A})₃ group is —Si(CH₃)₃).

In another embodiment, R¹ is phenyl substituted with 1-3 R²¹ groups independently selected from the group consisting of halos, —SF₅ and —OSF₅, wherein at least one R²¹ group is —SF₅ or —OSF₅.

In another embodiment, R¹ is phenyl substituted with 1-3 R²¹ groups independently selected from the group consisting of halos, —SF₅ and —OSF₅, wherein at least one R²¹ group is —SF₅ or —OSF₅.

In another embodiment, R¹ is phenyl substituted with 1-3 R²¹ groups independently selected from the group consisting of F, Cl, —SF₅ and —OSF₅.

In another embodiment, R¹ is phenyl substituted with 1-3 R²¹ groups independently selected from the group consisting of —SF₅ and —OSF₅.

In another embodiment, R¹ is phenyl substituted with 1-3 R²¹ groups independently selected from the group consisting of F, —SF₅ and —OSF₅, wherein at least one R²¹ group is —SF₅ or —OSF₅.

In another embodiment, R¹ is phenyl substituted with one —SF₅ group.

In another embodiment, R¹ is phenyl substituted with two —SF₅ groups.

In another embodiment, R¹ is phenyl substituted with three —SF₅ groups.

In another embodiment, R¹ is phenyl substituted with one —OSF₅ group.

In another embodiment, R¹ is phenyl substituted with two —OSF₅ groups.

In another embodiment, R¹ is phenyl substituted with three —OSF₅ groups.

In another embodiment, R¹ is phenyl substituted with 1 F.

In another embodiment, R¹ is phenyl substituted with 1 F, and also substituted with 1 to 2 groups independently selected from the group consisting of —SF₅ and —OSF₅.

In another embodiment R¹ is phenyl substituted with 2 F.

In another embodiment R¹ is phenyl substituted with 3F. In another embodiment of this invention L is selected from the group consisting of:

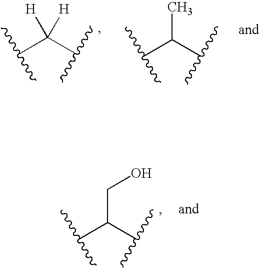

R¹ is selected from the group consisting of:

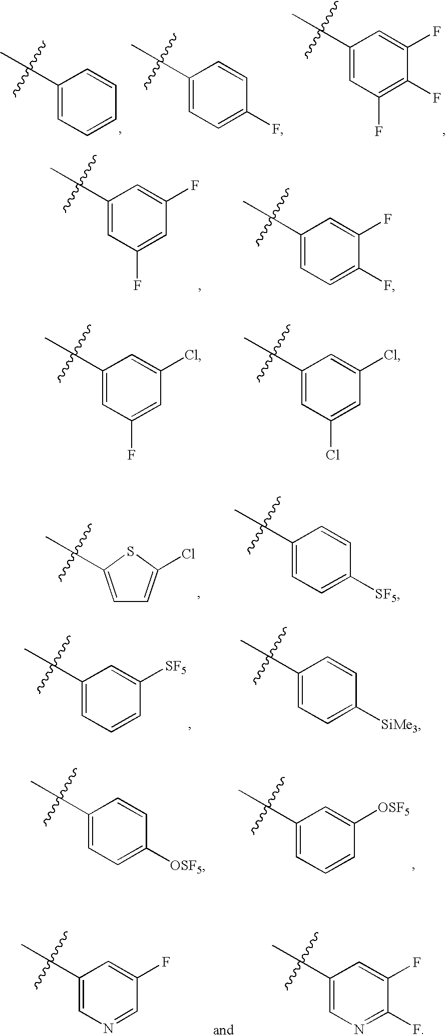

In another embodiment of this invention L is selected from the group consisting of:

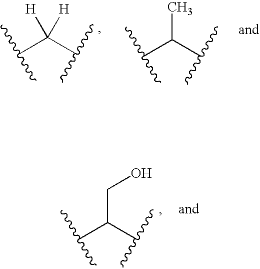

R¹ is selected from the group consisting of:

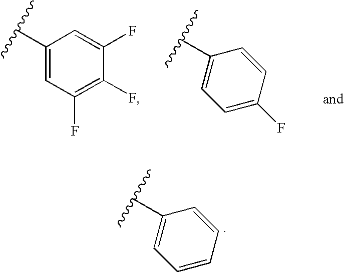

In another embodiment of this invention, the compound of formula (I) is selected from the group consisting of the compounds of formulas (IA), (IB), (IC), (ID), and (IE), L is selected from the group consisting of:

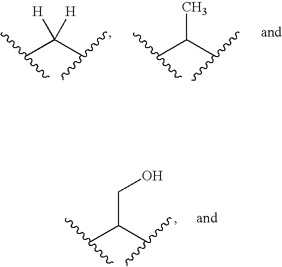

R¹ is selected from the group consisting of:

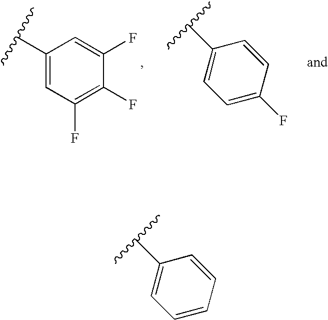

In another embodiment of this invention, the compound of formula (I) is the compounds of formula (IA), L is selected from the group consisting of:

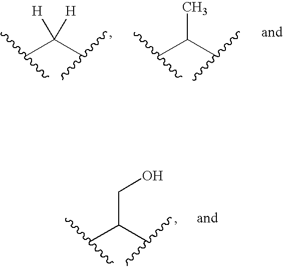

R¹ is selected from the group consisting of:

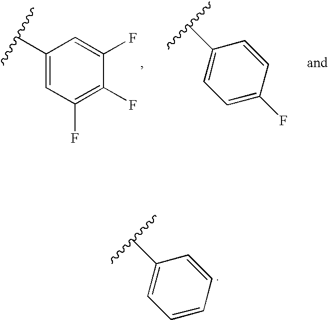

In another embodiment of this invention, the compound of formula (I) is the compound of formulas (IB), L is selected from the group consisting of:

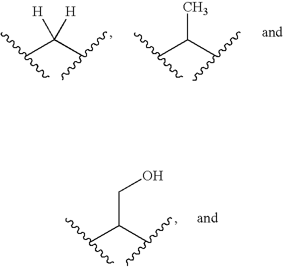

R¹ is selected from the group consisting of:

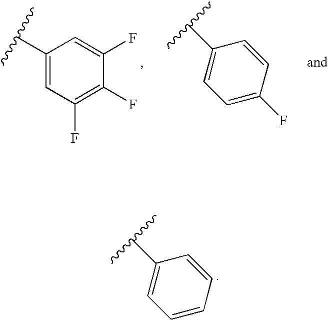

In another embodiment of this invention, the compound of formula (I) is the compounds of formula (IC), L is selected from the group consisting of:

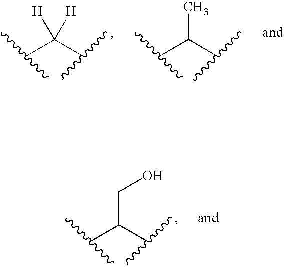

R¹ is selected from the group consisting of:

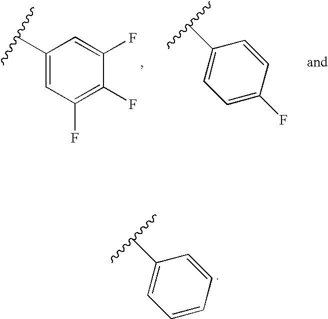

In another embodiment of this invention, the compound of formula (I) is the compounds of formula (ID), L is selected from the group consisting of:

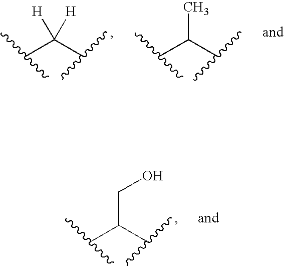

R¹ is selected from the group consisting of:

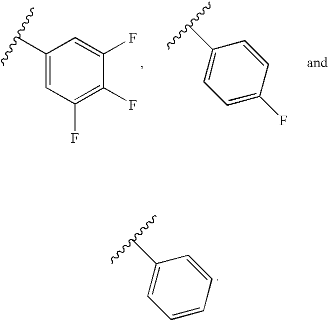

In another embodiment of this invention, the compound of formula (I) is the compounds of formula (IE), L is selected from the group consisting of:

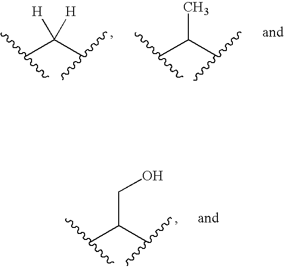

R¹ is selected from the group consisting of:

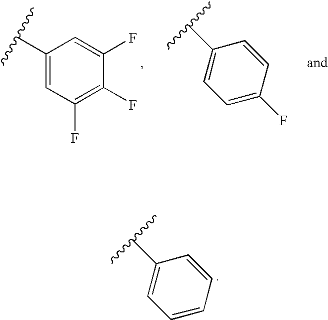

In another embodiment of this invention, R⁵ is taken together with R¹ and the carbon to which they are bound to form a heterocycloalkyl or heterocycloalkenyl ring fused to said R¹ ring, said fused ring is optionally substituted with 1 to 5 independently selected R²¹ groups.

In another embodiment of this invention, R⁵ is taken together with R¹ and the carbon to which they are bound to form a 5 to 7 membered heterocycloalkyl or heterocycloalkenyl ring fused to said R¹ ring, and wherein said heterocycloakyl and said heterocyicloalkenyl rings comprise 1 to 4 (including the atoms common to both rings) heteroatoms selected from the group consisting of: —N—, —O—, —S—, —S(O)—, and —S(O)₂—, and wherein said 5 to 7 membered ring is optionally substituted with 1 to 5 independently selected R²¹ groups.

In another embodiment of this invention, R⁶ is taken together with R¹ and the carbon to which they are bound to form a cycloakyl, cycloakenyl, heterocycloakyl or heterocycloalkenyl ring fused to said R¹ ring, said fused ring is optionally substituted with 1 to 5 independently selected R²¹ groups.

In another embodiment of this invention, R⁶ is taken together with R¹ and the carbon to which they are bound to form a 5 to 7 membered cycloalkyl, cycloalkenyl, heterocycloalkyl or heterocycloalkenyl ring fused to said R¹ ring, and wherein said heterocycloalkyl and said heterocylcloalkenyl rings comprise 1 to 4 (including the atoms common to both rings) heteroatoms selected from the group consisting of: —N—, —O—, —S—, —S(O)—, and —S(O)₂—, and wherein said 5 to 7 membered ring is optionally substituted with 1 to 5 independently selected R²¹ groups.

In another embodiment of this invention, Ring (B) is a cycloalkyl ring.

In another embodiment of this invention, Ring (B) is a cycloalkenyl ring.

In another embodiment of this invention, Ring (B) is a heterocycloalkyl ring.

In another embodiment of this invention, Ring (B) is a heterocycloalkenyl ring.

In another embodiment of this invention, Ring (B) is a phenyl ring.

In another embodiment of this invention, Ring (B) is a heteroaryl ring.

In another embodiment of this invention Ring (B) is a cycloalkyl ring wherein B¹ to B⁴ are carbon.

In another embodiment of this invention Ring (B) is a cycloalkyl ring wherein B¹ is carbon, one of B², B³, or B⁴ is C and the remaining two are selected from the group consisting of: —(C═O)— and —(C═NR^{21A})—(e.g., —(C═N—OR¹⁵)—, and —(C═N—N(R¹⁵)(R¹⁶))—)

In another embodiment of this invention Ring (B) is a cycloalkyl ring wherein B¹ is carbon, two of B², B³, or B⁴ are C and the remaining one is selected from the group consisting of: —(C═O)— and —(C═NR^{21A})—(e.g., —(C═N—OR¹⁵)—, and —(C═N—N(R¹⁵)(R¹⁶))-).

In another embodiment of this invention Ring (B) is a heterocycloalkyl ring wherein one of B², B³, or B⁴ is selected from the group consisting of: —(C═O)— and —(C═NR^{21A})— (e.g., —(C═N—OR¹⁵)—, and -(C═N—N(R¹⁵)(R¹⁶))-).

In another embodiment of this invention Ring (B) is a heterocycloalkenyl ring wherein one of B², B³, or B⁴ is selected from the group consisting of: —(C═O)— and —(C═NR^{21A})— (e.g., —(C═N—OR¹⁵)—, and -(C═N—N(R¹⁵)(R¹⁶))-).

In another embodiment of this invention L is a direct bond.

In another embodiment of this invention L is —O.

In another embodiment of this invention L is —NR⁵—.

In another embodiment of this invention L is —S—.

In another embodiment of this invention L is —SO—.

In another embodiment of this invention L is —S(O)₂—.

In another embodiment of this invention L is —(C═O)—.

In another embodiment of this invention L is —(C═NR^{21A})—

In another embodiment B¹ is CH.

In another embodiment B¹ is C.

In another embodiment B¹ is N.

In another embodiment of this invention R³ is phenyl.

In another embodiment of this invention R³ is phenyl substituted with 1 to 3 independently selected R²¹ groups.

In another embodiment of this invention R³ is phenyl substituted with 1 R²¹ group.

In another embodiment of this invention R³ is phenyl substituted with 1 R²¹ group wherein said R²¹ group is halo.

In another embodiment of this invention R³ is phenyl substituted with 1 R²¹ group wherein said R²¹ group is halo, and said halo is F.

In another embodiment of this invention R³ is phenyl substituted with 1 R²¹ group, wherein said R²¹ group is —OR¹⁵.

In another embodiment of this invention R³ is phenyl substituted with 1 R²¹ group, wherein said R²¹ group is —OR¹⁵, and wherein said R¹⁵ is alkyl (e.g., methyl).

In another embodiment of this invention R³ is pyridyl.

In another embodiment of this invention R³ is pyridyl substituted with 1 to 3 independently selected R²¹ groups.

In another embodiment of this invention R⁴ is heteroaryl.

In another embodiment of this invention R⁴ is heteroaryl substituted with 1 to 3 independently selected R²¹ groups.

In another embodiment of this invention R⁴ is heteroaryl substituted with 1 R²¹ group.

In another embodiment of this invention R⁴ is heteroaryl substituted with 1 to 3 independently selected R²¹ groups, wherein said R²¹ groups are the same or different alkyl group.

In another embodiment of this invention R⁴ is heteroaryl substituted with 1 R²¹ group, wherein said R²¹ group is alkyl (e.g., methyl).

In another embodiment of this invention R⁴ is selected from the group consisting of:

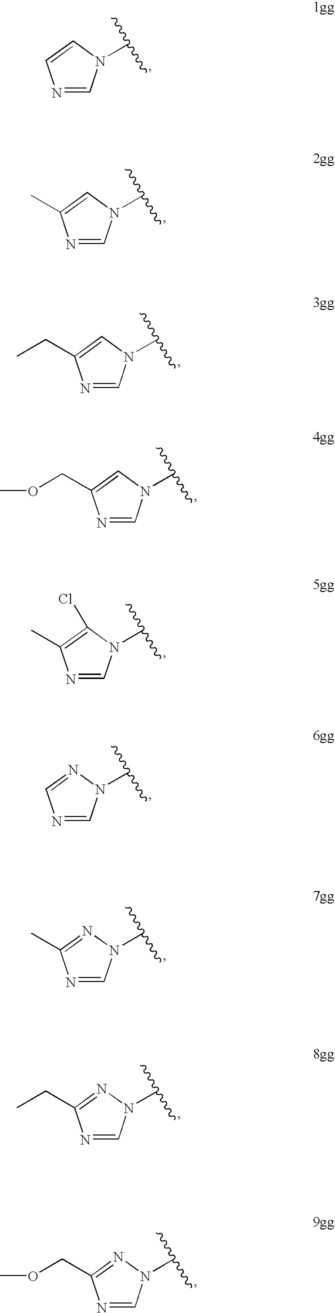

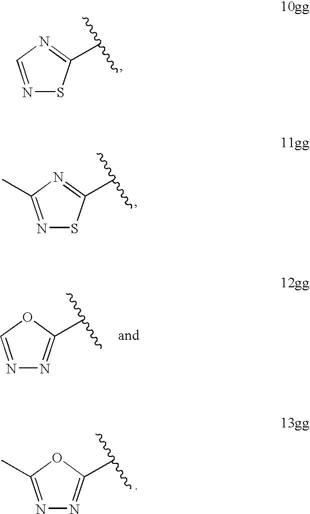

In another embodiment of this invention R⁴ is imidazolyl.

In another embodiment of this invention R⁴ is the imidazoyl:

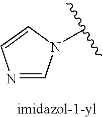

In another embodiment of this invention R⁴ is imidazolyl substituted with 1 to 3 independently selected R²¹ groups.

In another embodiment of this invention R⁴ is imidazolyl substituted with 1 R²¹ group.

In another embodiment of this invention R⁴ is imidazolyl substituted with 1 to 3 independently selected R²¹ groups, wherein said R²¹ groups are the same or different alkyl group.

In another embodiment of this invention R⁴ is imidazolyl substituted with 1 R²¹ group, wherein said R²¹ group is alkyl (e.g., methyl).

In another embodiment of this invention R⁴ is:

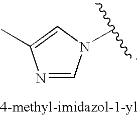

Examples of the R⁴—R³— moiety include, but are not limited to:

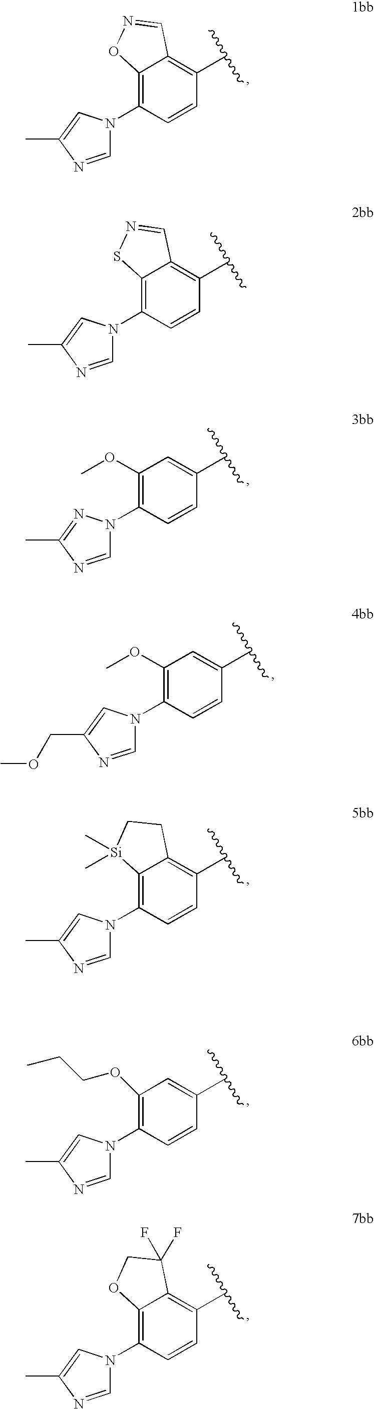

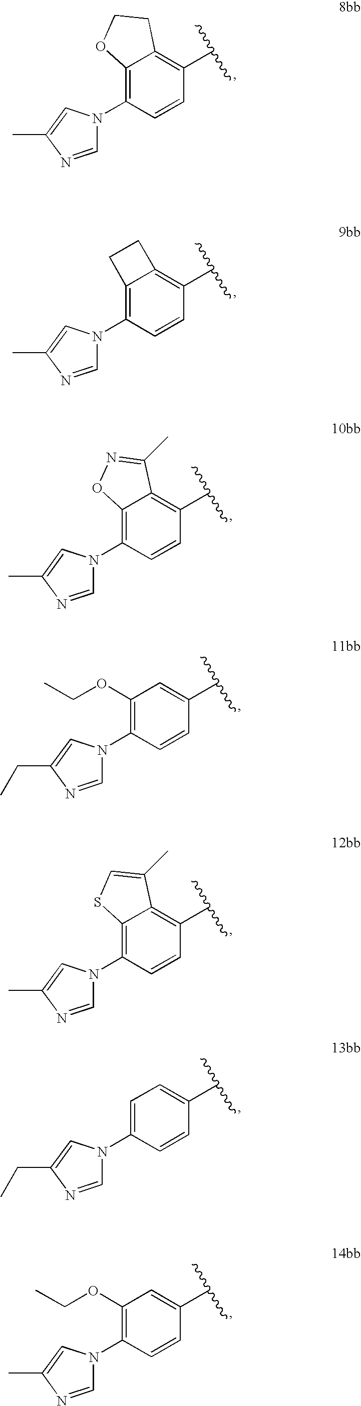

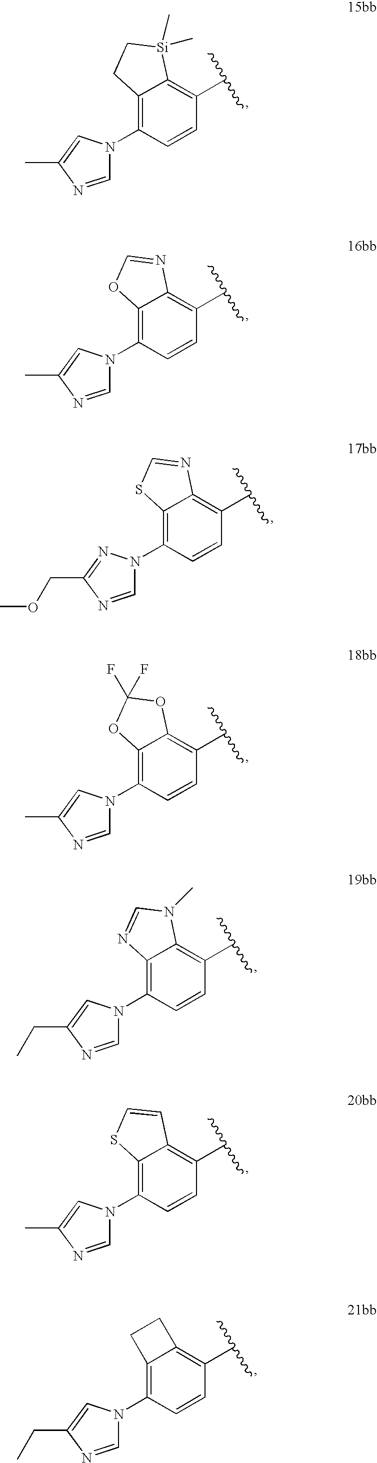

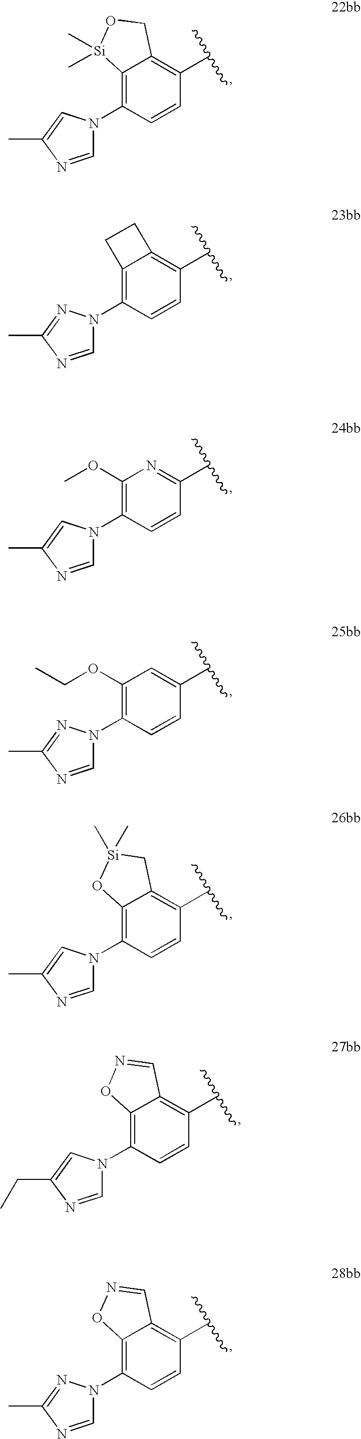

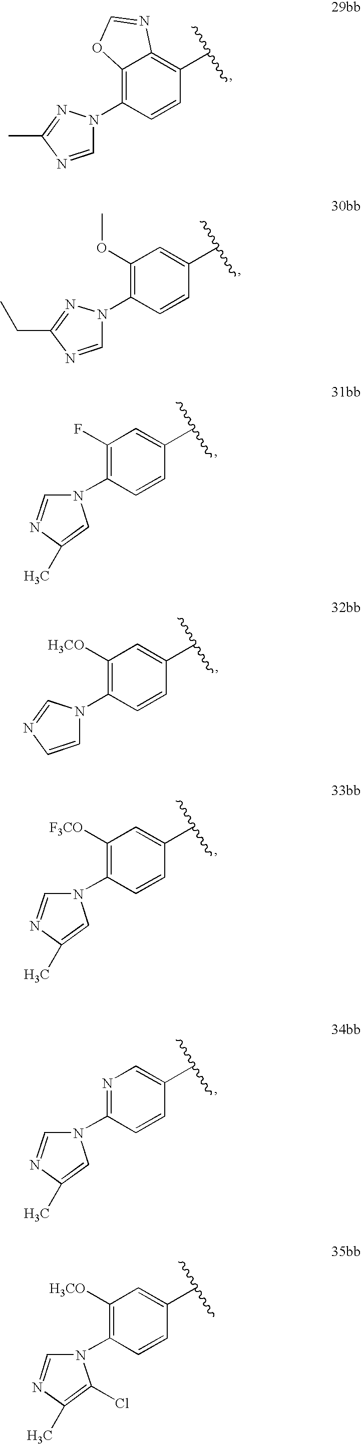

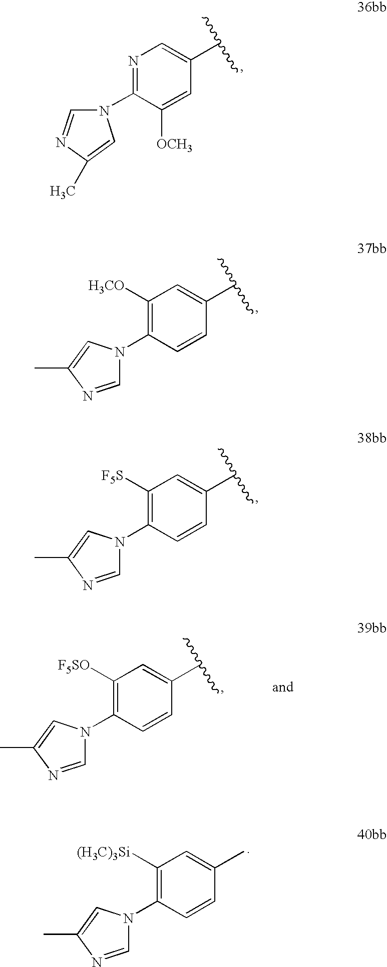

In another embodiment the R⁴—R³— moiety is 1 bb. In another embodiment the R⁴—R³— moiety is 2 bb. In another embodiment the R⁴—R³— moiety is 3 bb. In another embodiment the R⁴—R³— moiety is 4 bb. In another embodiment the R⁴—R³— moiety is 5 bb. In another embodiment the R⁴—R³— moiety is 6 bb, In another embodiment the R⁴—R³— moiety is 7 bb. In another embodiment the R⁴—R³— moiety is 8 bb, in another embodiment the R⁴—R³— moiety is 9 bb. In another embodiment the R⁴—R³— moiety is 10 bb. In another embodiment the R⁴—R³— moiety is 1 bb. In another embodiment the R⁴—R³— moiety is 12 bb. In another embodiment the R⁴—R³— moiety is 13 bb. In another embodiment the R⁴—R³— moiety is 14 bb, in another embodiment the R⁴—R³— moiety is 15 bb. In another embodiment the R⁴—R³— moiety is 16 bb, In another embodiment the R⁴—R³— moiety is 17 bb. In another embodiment the R⁴—R³— moiety is 18 bb. In another embodiment the R⁴—R³— moiety is 19 bb. In another embodiment the R⁴—R³— moiety is 20 bb. In another embodiment the R⁴—R³— moiety is 21 bb. In another embodiment the R⁴—R³— moiety is 22 bb. In another embodiment the R⁴—R³— moiety is 23 bb. In another embodiment the R⁴—R³— moiety is 24 bb. In another embodiment the R⁴—R³— moiety is 25 bb. In another embodiment the R⁴—R³— moiety is 26 bb. In another embodiment the R⁴—R³— moiety is 27 bb. In another embodiment the R⁴—R³— moiety is 28 bb. In another embodiment the R⁴—R³— moiety is 29 bb. In another embodiment the R⁴—R³— moiety is 30 bb. In another embodiment the R⁴—R³— moiety is 31 bb. In another embodiment the R⁴—R³— moiety is 32 bb. In another embodiment the R⁴—R³— moiety is 33 bb. In another embodiment the R⁴—R³— moiety is 34 bb. In another embodiment the R⁴—R³— moiety is 35 bb. In another embodiment the R⁴—R³— moiety is 36 bb. In another embodiment the R⁴—R³— moiety is 37 bb. In another embodiment the R⁴—R³— moiety is 38 bb. In another embodiment the R⁴—R³— moiety is 39 bb. In another embodiment the R⁴—R³— moiety is 40 bb.

In another embodiment of the invention:
R³ is selected from the group consisting of: (1) heteroaryl and (2) hetereoaryl substituted with 1 to 3 independently selected R²¹ groups; and
R⁴ is selected from the group consisting of: (1) heteroaryl (e.g., imidazolyl, such as, for example imidazol-1-yl), (2) heteroaryl (e.g., imidazolyl, such as, for example imidazol-1-yl) substituted with 1 to 3 independently selected R²¹ groups, (3) heteroaryl (e.g., imidazolyl, such as, for example imidazol-1-yl) substituted with 1 R²¹ group, (4) heteroaryl (e.g., imidazolyl, such as, for example imidazol-1-yl) substituted with 1 to 3 independently selected R²¹ groups, wherein said R²¹ groups are the same or different alkyl group, and (5) heteroaryl (e.g., imidazolyl, such as, for example imidazol-1-yl) substituted with 1 R²¹ group, wherein said R²¹ group is alkyl (e.g., methyl).

In another embodiment of this invention the —R³—R⁴ moiety is:

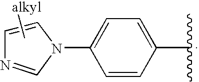

In another embodiment of this invention the —R³—R⁴ moiety is:

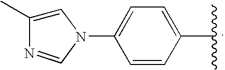

In another embodiment of this invention the —R³—R⁴ moiety is:

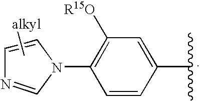

In another embodiment of this invention the —R³—R⁴ moiety is:

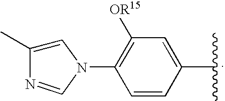

In another embodiment of this invention the —R³—R⁴ moiety is:

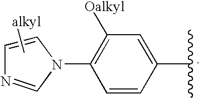

In another embodiment of this invention the —R³—R⁴ moiety is:

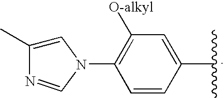

In another embodiment of this invention the —R³—R⁴ moiety is:

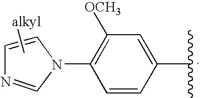

In another embodiment of this invention the —R³—R⁴ moiety is:

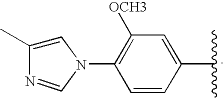

in another embodiment of this invention the —R³—R⁴ moiety is:

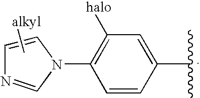

In another embodiment of this invention the —R³—R⁴ moiety is:

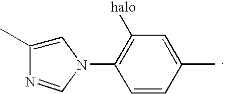

In another embodiment of this invention the —R³—R⁴ moiety is:

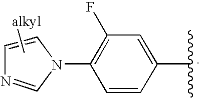

In another embodiment of this invention the —R³—R⁴ moiety is:

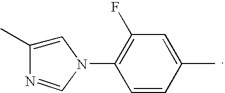

In another embodiment of this invention the —R³—R⁴ moiety is:

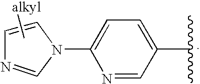

In another embodiment of this invention the —R³—R⁴ moiety is:

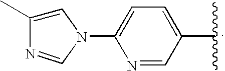

In another embodiment of this invention the —R³—R⁴ moiety is:

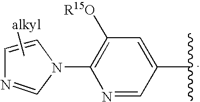

In another embodiment of this invention the —R³—R⁴ moiety is:

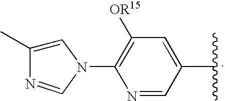

In another embodiment of this invention the —R³—R⁴ moiety is:

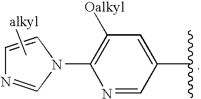

in another embodiment of this invention the —R³—R⁴ moiety is:

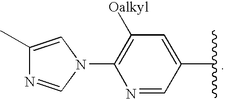

In another embodiment of this invention the —R³—R⁴ moiety is:

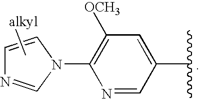

In another embodiment of this invention the —R—R⁴ moiety is:

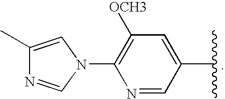

In another embodiment of this invention the —R³—R⁴ moiety is:

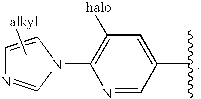

In another embodiment of this invention the —R³—R⁴ moiety is:

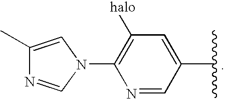

In another embodiment of this invention the —R³—R⁴ moiety is:

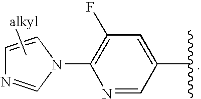

In another embodiment of this invention the —R³—R⁴ moiety is:

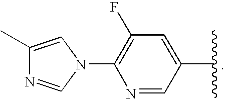

In another embodiment of this invention R¹ is H.

In another embodiment of this invention R¹ is alkyl.

In another embodiment of this invention R¹ is aryl In another embodiment of this invention R¹ is aryl substituted with 1 to 3 independently selected R²¹ groups.

In another embodiment of this invention R¹ is aryl substituted with 1 to 3 independently selected R²¹ groups wherein said R²¹ groups are halo.

In another embodiment of this invention R¹ is aryl substituted with 1 to 3 independently selected R²¹ groups wherein said R²¹ groups are F.

In another embodiment of this invention R¹ is aryl substituted with 1 R²¹ group.

In another embodiment of this invention R¹ is aryl substituted with 2 R²¹ groups.

In another embodiment of this invention R¹ is aryl substituted with 3 R²¹ groups.

In another embodiment of this invention R¹ is aryl substituted with 1 R² group wherein said R²¹ group is halo.

In another embodiment of this invention R¹ is aryl substituted with 2 R²¹ groups wherein said R²¹ groups are the same or different halo.

In another embodiment of this invention R¹ is aryl substituted with 3 R²¹ groups wherein said R²¹ groups are the same or different halo.

In another embodiment of this invention R¹ is phenyl substituted with 1 to 3 independently selected R²¹ groups.

In another embodiment of this invention R¹ is phenyl substituted with 1 to 3 independently selected R²¹ groups wherein said R²¹ groups are halo.

In another embodiment of this invention R¹ is phenyl substituted with 1 to 3 independently selected R²¹ groups wherein said R²¹ groups are F.

In another embodiment of this invention R¹ is

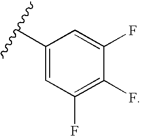

In another embodiment of this invention R¹ is phenyl substituted with 1 R² group.

In another embodiment of this invention R¹ is phenyl substituted with 2 R²¹ groups.

In another embodiment of this invention R¹ is phenyl substituted with 3 R²¹ groups

In another embodiment of this invention R¹ is phenyl substituted with 1 R²¹ group wherein said R²¹ group is halo.

In another embodiment of this invention R¹ is phenyl substituted with 2 R²¹ groups wherein said R²¹ groups are the same or different halo.

In another embodiment of this invention R¹ is phenyl substituted with 3 R²¹ groups wherein said R²¹ groups are the same or different halo.

In another embodiment of this invention R¹ is 4-F-phenyl.

In another embodiment of this invention the -L-R¹ moiety is:

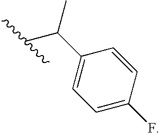

In another embodiment of this invention the -L-R¹ moiety is:

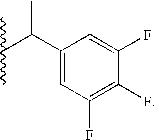

In another embodiment of this invention the -L-R¹ moiety is:

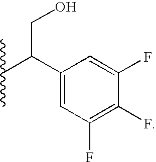

In another embodiment of this invention the -L-R¹ moiety is:

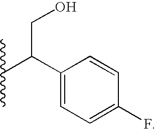

In another embodiment of this invention the -L-R¹ moiety is selected from the group consisting of:

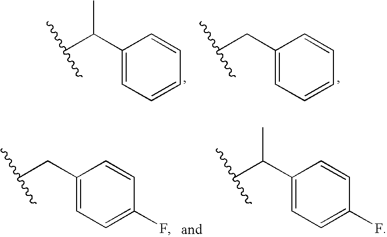

In another embodiment of this invention the -L-R¹ moiety is selected from the group consisting of:

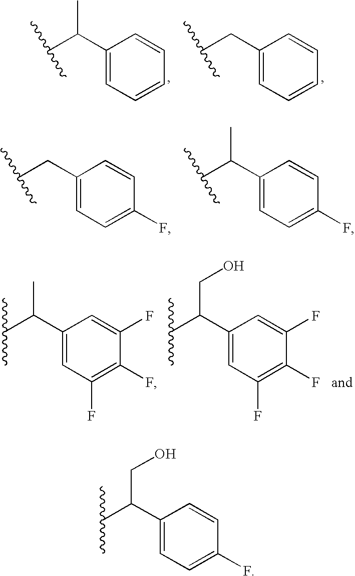

In another embodiment of this invention R³ is selected from the group consisting of phenyl and phenyl substituted with one or more R²¹ groups, and said R⁴ group is selected from the group consisting of heteroaryl and heteroaryl substituted with one or more R²¹ groups, and wherein each R²¹ is independently selected.

In another embodiment of this invention: (a) L is —C(R⁶)(R⁷)— wherein R⁶ and R⁷ are independently selected from the group consisting of H and alkyl (e.g., methyl), and in one example one of R⁶ and R⁷ is H and the other is alkyl (e.g., methyl), and in another example both R⁶ and R⁷ are H, (b) R¹ is aryl (e.g. phenyl) substituted with 1 to 3 independently selected R²¹ groups wherein said R²¹ groups are halo (e.g., F), and in one example R¹ is phenyl substituted with two F, and in another example R¹ is phenyl substituted with 1 F, (c) R³ is selected from the group consisting of phenyl and phenyl substituted with one or more independently selected R²¹ groups, and (d) R⁴ is selected from the group consisting of heteroaryl and heteroaryl substituted with one or more independently selected R²¹ groups.

In another embodiment of this invention: (a) L is —C(R⁶)(R⁷)— wherein R⁶ and R⁷ are independently selected from the group consisting of H and alkyl (e.g., methyl), and in one example one of R⁶ and R⁷ is H and the other is alkyl (e.g., methyl), and in another example both R⁶ and R⁷ are H, (b) R¹ is aryl (e.g. phenyl) substituted with 1 to 3 independently selected R²¹ groups wherein said R² groups are halo (e.g., F), and in one example R¹ is phenyl substituted with two F, and in another example R¹ is phenyl substituted with 1 F, (c) R³ is selected from the group consisting of phenyl and phenyl substituted with one or more independently selected R²¹ groups, and (d) R⁴ is selected from the group consisting of imidazoyl and imidazoyl substituted with one or more independently selected R²¹ groups.

In another embodiment of this invention: (a) L is —C(R⁶)(R⁷)— wherein R⁶ and R⁷ are independently selected from the group consisting of H and alkyl (e.g., methyl), and in one example one of R⁶ and R⁷ is H and the other is alkyl (e.g., methyl), and in another example both R⁶ and R⁷ are H, (b) R¹ is aryl (e.g. phenyl) substituted with 1 to 3 independently selected R²¹ groups wherein said R²¹ groups are halo (e.g., F), and in one example R¹ is phenyl substituted with two F, and in another example R¹ is phenyl substituted with 1 F, (c) R³ is selected from the group consisting of phenyl and phenyl substituted with one or more independently selected —OR¹⁵ groups, and (d) R⁴ is selected from the group consisting of imidazolyl and imidazolyl substituted with one or more independently selected alkyl groups groups.

In another embodiment of this invention: (a) L is —C(R⁶)(R⁷)— wherein R³ and R⁴ are independently selected from the group consisting of H and alkyl (e.g., methyl), and in one example one of R⁶ and R⁷ is H and the other is alkyl (e.g., methyl), and in another example both R⁶ and R⁷ are H, (b) R¹ is aryl (e.g. phenyl) substituted with 1 to 3 independently selected R²¹ groups wherein said R²¹ groups are halo (e.g., F), and in one example R¹ is phenyl substituted with two F, and in another example R¹ is phenyl substituted with 1 F, (c) R³ is selected from the group consisting of phenyl and phenyl substituted with one or two independently selected —OR¹⁵ groups, wherein R¹⁵ is alkyl, and (d) R⁴ is selected from the group consisting of imidazolyl and imidazolyl substituted with one or two independently selected alkyl groups groups.

In another embodiment of this invention: (a) L is —C(R⁶)(R⁷)— wherein R³ and R⁴ are independently selected from the group consisting of H and alkyl (e.g., methyl), and in one example one of R⁶ and R⁷ is H and the other is alkyl (e.g., methyl), and in another example both R⁶ and R⁷ are H, (b) R¹ is aryl (e.g. phenyl) substituted with 1 to 3 independently selected R²¹ groups wherein said R²¹ groups are halo (e.g., F), and in one example R¹ is phenyl substituted with two F, and in another example R¹ is phenyl substituted with 1 F, (c) R³ is selected from the group consisting of phenyl and phenyl substituted with one or two independently selected —OR¹⁵ groups, wherein R¹⁵ is methyl, and (d) R⁴ is selected from the group consisting of imidazolyl and imidazolyl substituted with one or two independently selected methyl groups groups.

In another embodiment of this invention: (a) L is —C(R⁶)(R⁷)— wherein R⁶ and R⁷ are independently selected from the group consisting of H and alkyl (e.g., methyl), and in one example one of R⁶ and R⁷ is H and the other is alkyl (e.g., methyl), and in another example both R⁶ and R⁷ are H, (b) R¹ is aryl (e.g. phenyl) substituted with 1 to 3 independently selected R²¹ groups wherein said R²¹ groups are halo (e.g., F), and in one example R¹ is phenyl substituted with two F, and in another example R¹ is phenyl substituted with 1 F, (c) R³ is phenyl substituted with one-OR¹⁵ group, wherein R¹⁵ is methyl, and (d) R⁴ is selected from the group consisting of imidazolyl and imidazolyl substituted with one methyl group.

In another embodiment of this invention the -L-R¹ moiety is selected from the group consisting of:

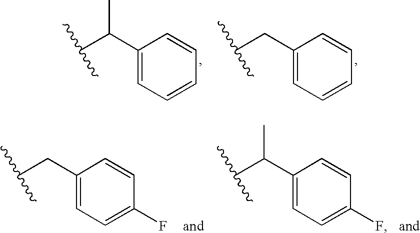

the R⁴—R— moiety is:

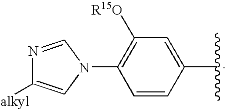

In another embodiment of this invention the -L-R¹ moiety is selected from the group consisting of:

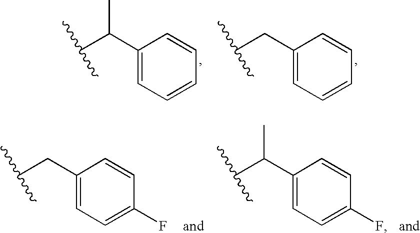

the R—R⁴— moiety is:

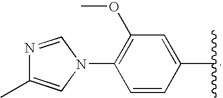

In another embodiment of this invention: (a) L is —C(R⁶)(R⁷)— wherein R⁶ and R⁷ are independently selected from the group consisting of H and alkyl (e.g., methyl), and in one example one of R⁶ and R⁷ is H and the other is alkyl (e.g., methyl), and in another example both R³ and R⁴ are H, (b) R¹ is aryl (e.g. phenyl) substituted with 1 to 3 independently selected R²¹ groups wherein said R²¹ groups are halo (e.g., F), and in one example R¹ is phenyl substituted with two F, and in another example R¹ is phenyl substituted with 1 F, (c) R³ is selected from the group consisting of phenyl and phenyl substituted with one or two independently selected —OR¹⁵ groups, wherein R¹⁵ is methyl, and (d) R⁴ is selected from the group consisting of imidazolyl and imidazolyl substituted with one or two independently selected methyl groups groups.

In another embodiment of this invention: (a) L is —C(R⁶)(R⁷)— wherein R⁶ and R⁷ are independently selected from the group consisting of H and alkyl (e.g., methyl), and in one example one of R⁶ and R⁷ is H and the other is alkyl (e.g., methyl), and in another example both R⁶ and R⁷ are H, (b) R¹ is aryl (e.g. phenyl) substituted with 1 to 3 independently selected R²¹ groups wherein said R²¹ groups are halo (e.g., F), and in one example R¹ is phenyl substituted with two F, and in another example R¹ is phenyl substituted with 1 F, (c) R³ is phenyl substituted with one —OR¹⁵ group, wherein R¹⁵ is methyl, and (d) R⁴ is selected from the group consisting of imidazolyl and imidazolyl substituted with one methyl group.

In another embodiment of this invention the -L-R′ moiety is selected from the group consisting of:

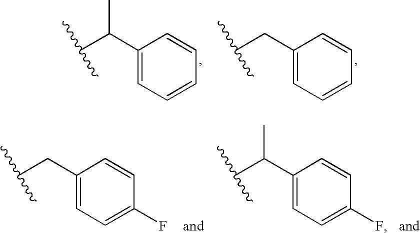

the R⁴—R³— moiety is:

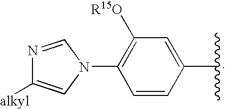

In another embodiment of this invention the -L-R′ moiety is selected from the group consisting of:

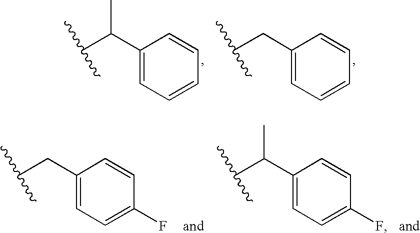

the R⁴—R³— moiety is:

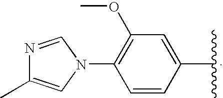

In another embodiment the -L R¹ moiety is selected from the group consisting of:

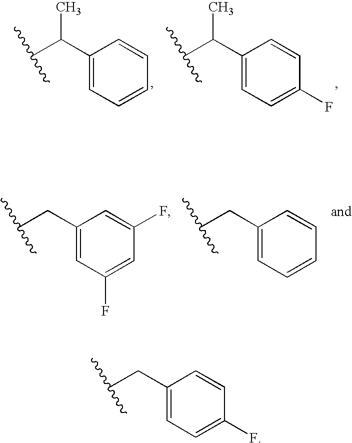

Other embodiments of this invention are directed to compounds of formula (I) wherein R³ is phenyl or phenyl substituted with one or more (e.g., one or two, or one) R²¹ groups (e.g., —OR¹⁵, wherein, for example, R¹⁵ is alkyl, such as, for example, methyl), and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

Thus, examples of the

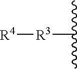

moiety of the compounds of this invention include, but are not limited to:

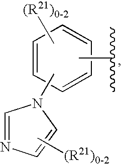

such as, for example,

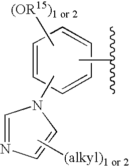

wherein R¹⁵ is alkyl (e.g., methyl), such as, for example,

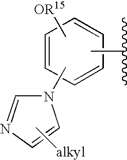

wherein R¹⁵ is alkyl (e.g., methyl), such as, for example,

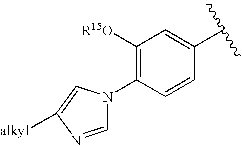

wherein R¹⁵ is alkyl (e.g., methyl), such as, for example,

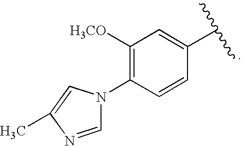

Representative (A) and (B) fused rings for formula (I) include but are not limited to:

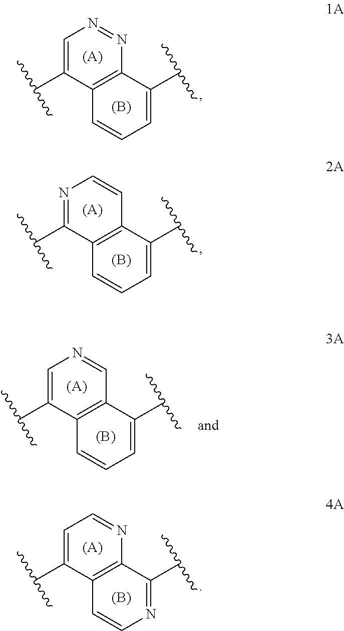

Compounds of formula (I) include but are not limited to:

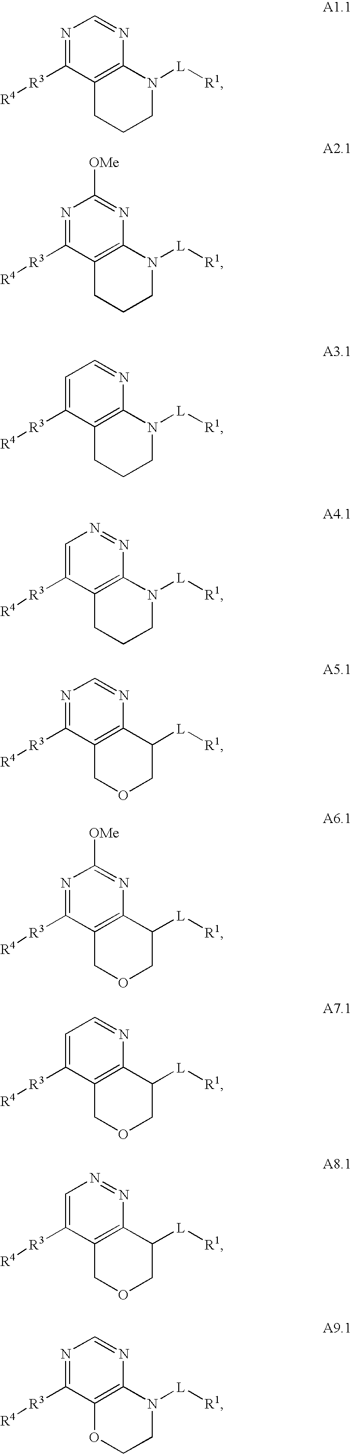

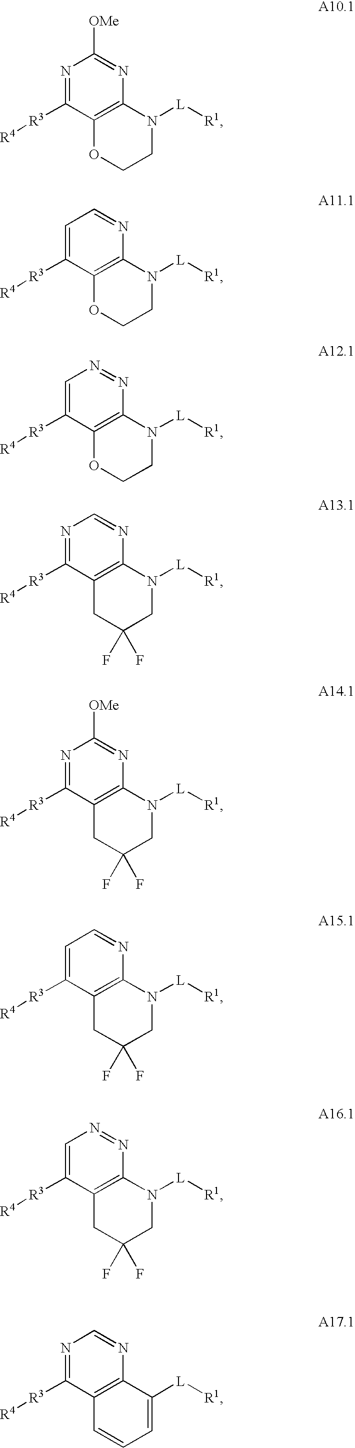

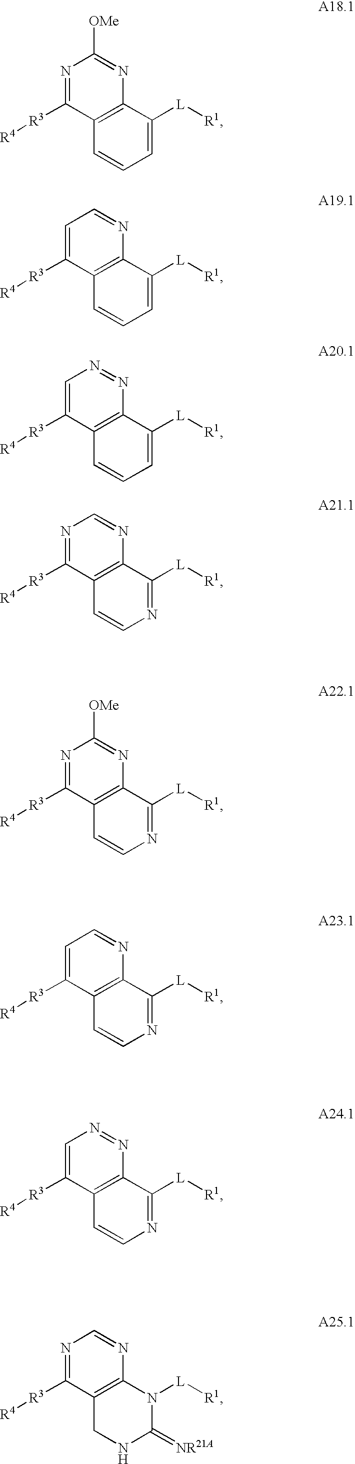

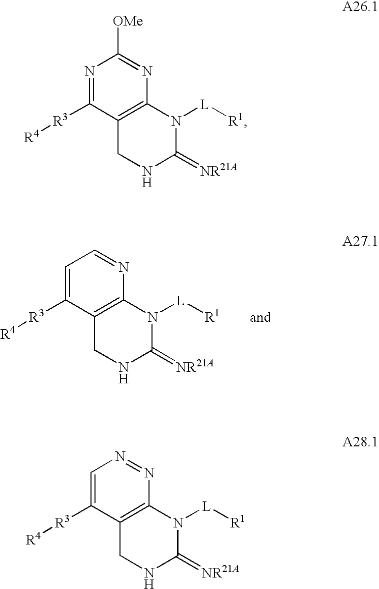

wherein R³, R⁴, L, R¹ and R^{21A} are as defined for formula (I) and the embodiments thereof.

Representative (A) and (B) fused rings for formula (I) also include but are not limited to:

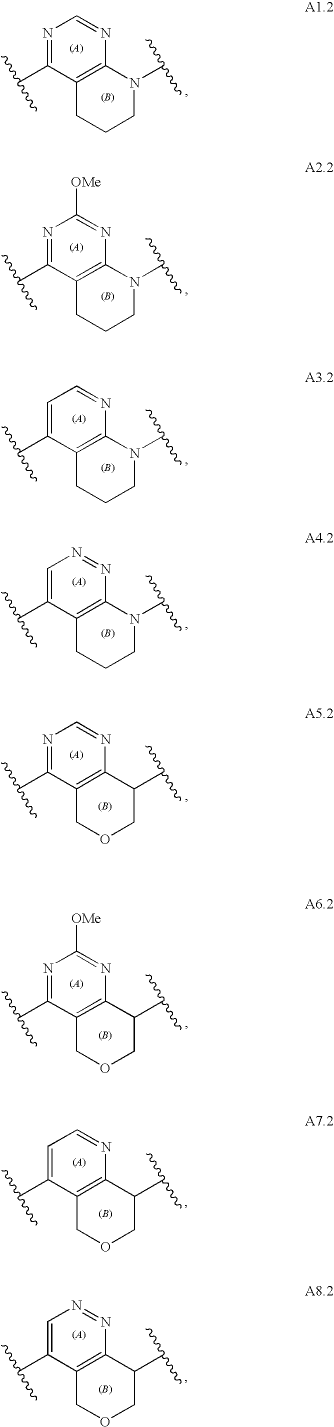

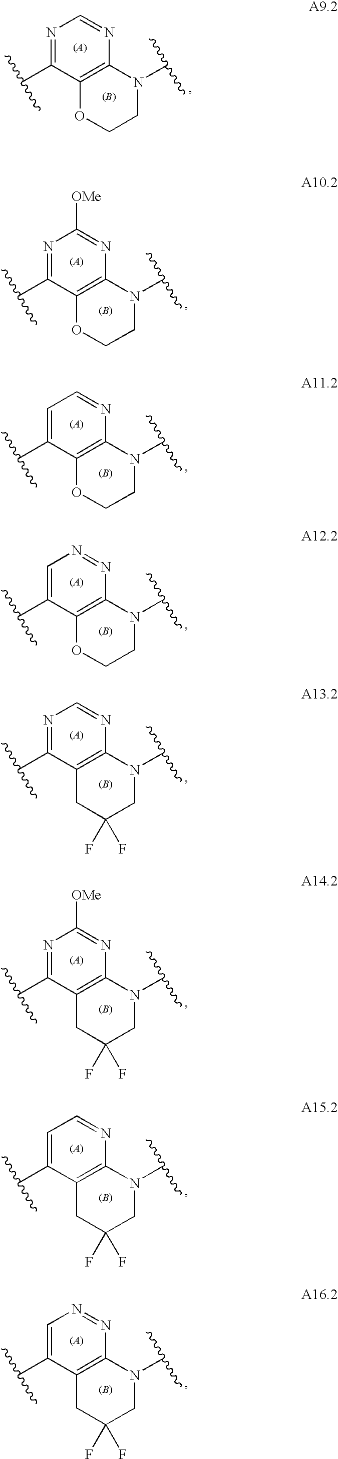

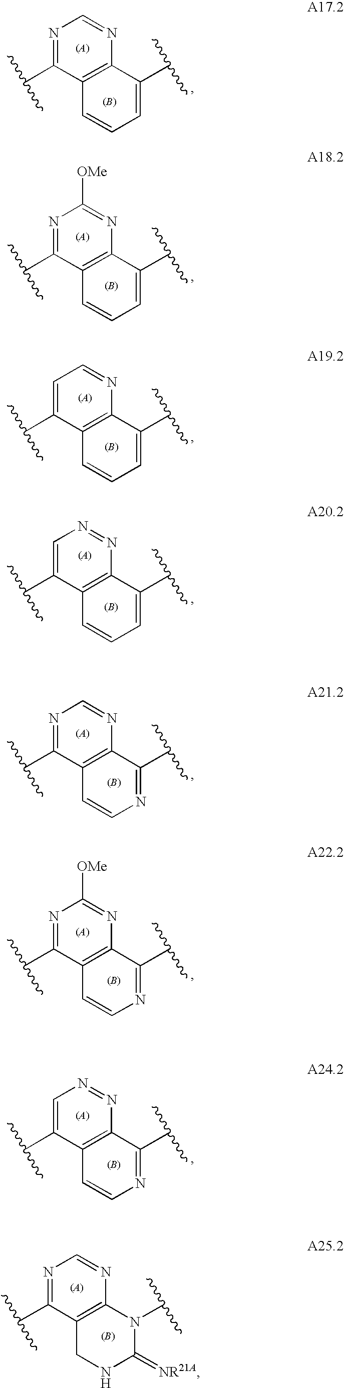

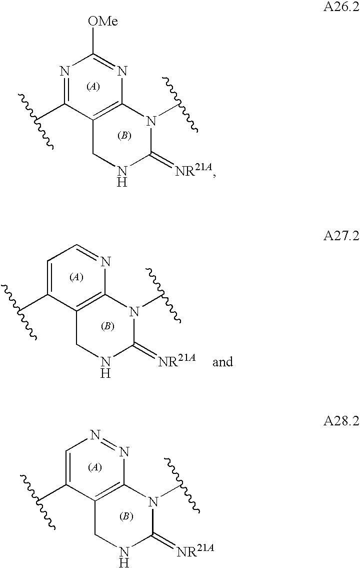

wherein R^{21A} is as defined for formula (I) and the embodiments thereof.

Representative compounds of this invention include, but are not limited to:

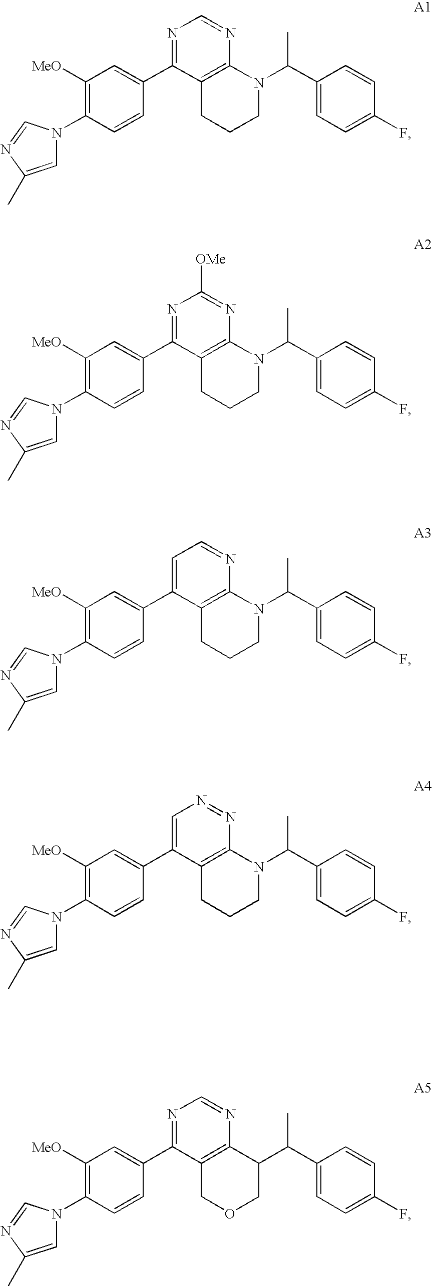

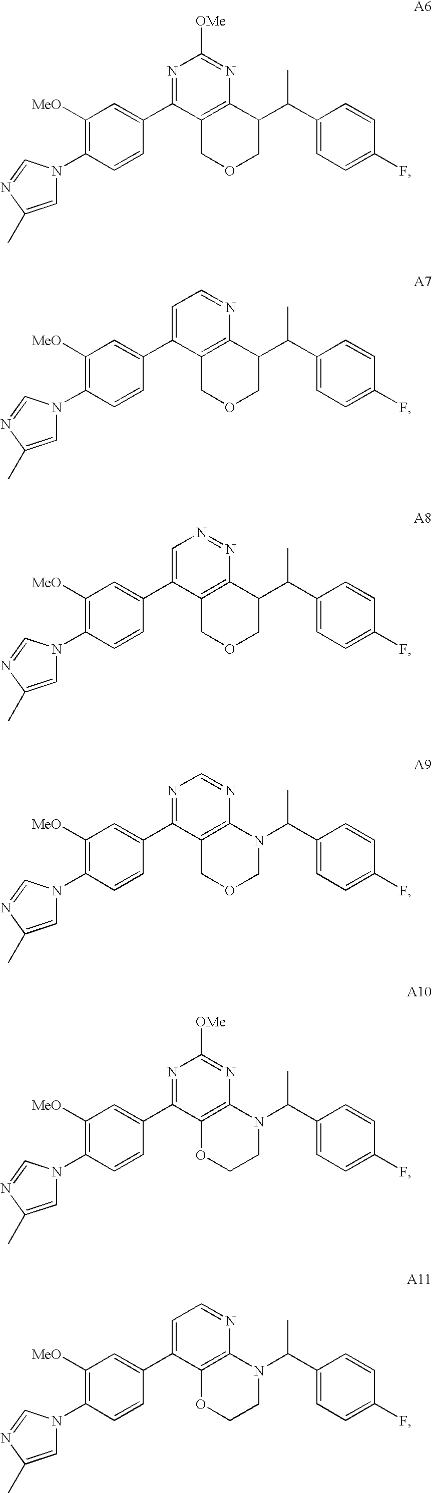

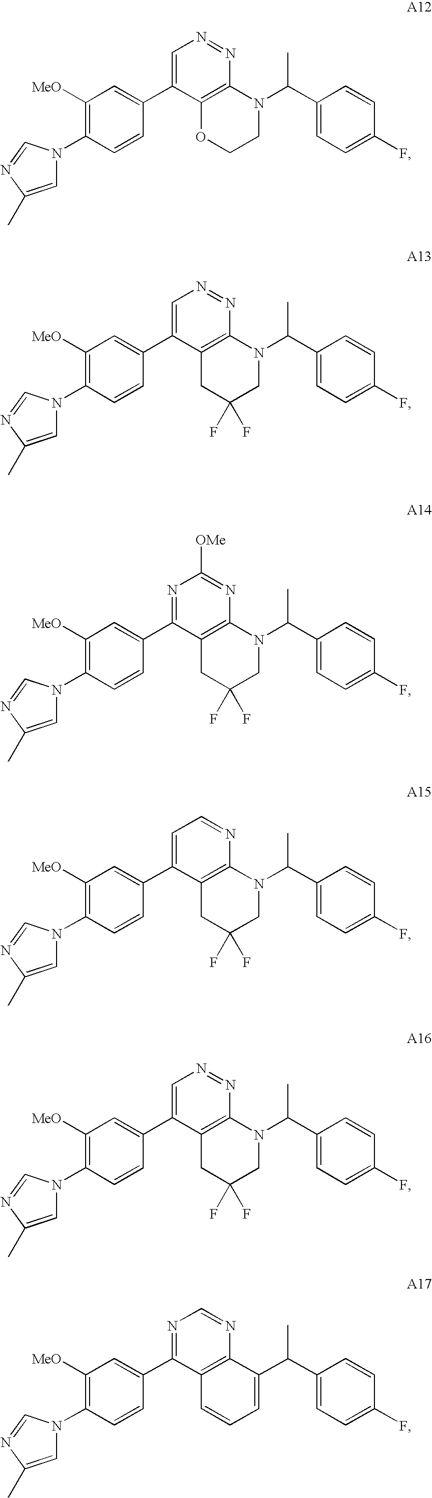

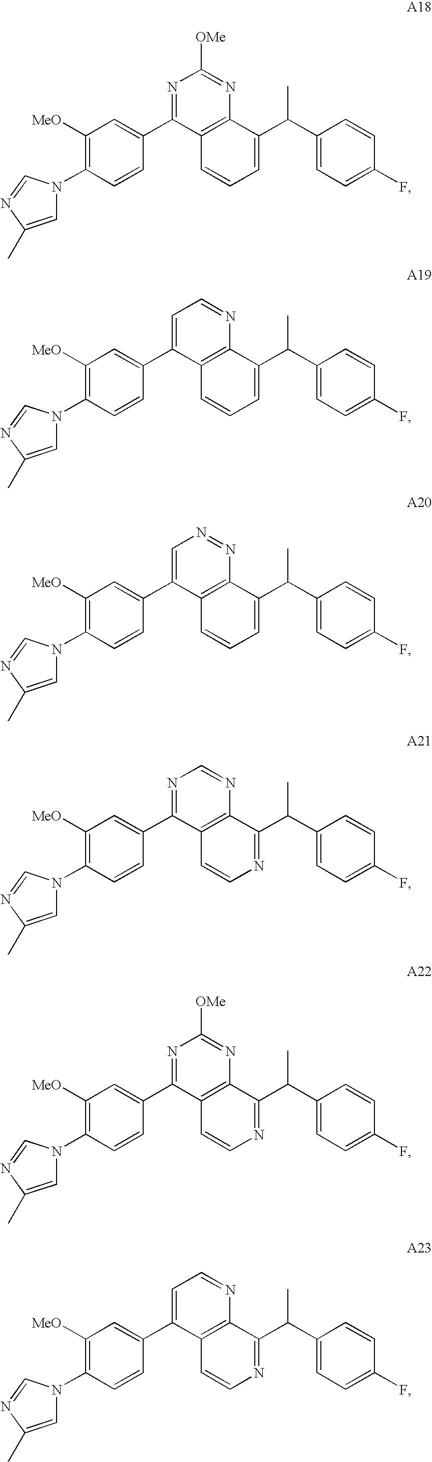

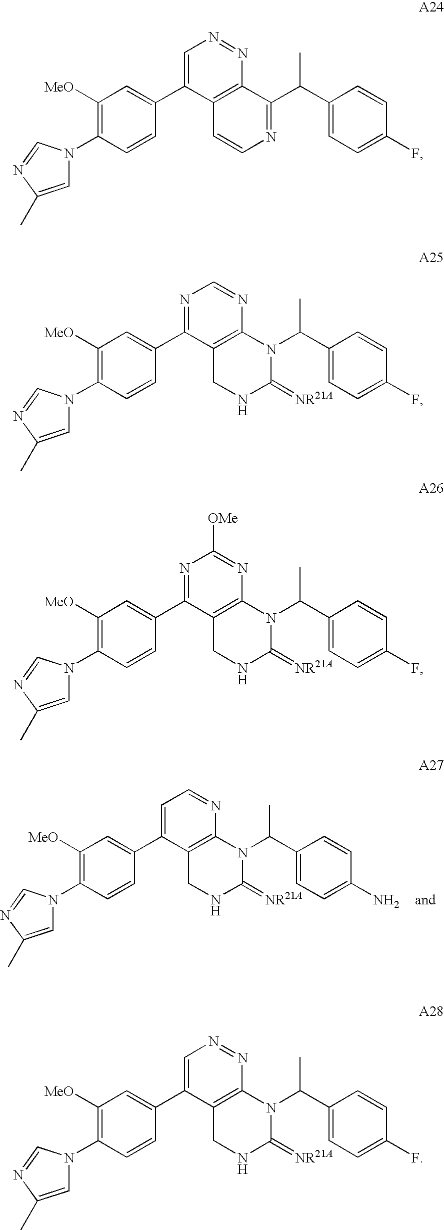

Other embodiments of this invention are directed to any of the embodiments above that are directed to L, R¹, R³, and R⁴ (or any combinations thereof) wherein the fused rings are selected from the group consisting of: 1A to 4A.

Other embodiments of this invention are directed to any of the embodiments above that are directed to L, R¹, R³, and R⁴ (or any combinations thereof) wherein the fused rings are selected from the group consisting of: A1.2 to A22.2, and A24.2 to A28.2.

Another embodiment of this invention is directed to a compound of formula (I) selected from the group consisting of: compounds IA to IE, 1A to 4A, A1.1 to A28.1, A1.2 to A22.2, A24.2 to A28.2, 5.1, 8.1, 11.1, and A1 to A28.

Another embodiment of this invention is directed to a compound of formula (I) selected from the group consisting of: compounds IA to IE.

Another embodiment of this invention is directed to a compound of formula (I) selected from the group consisting of: compounds 1A to 4A.

Another embodiment of this invention is directed to a compound of formula (I) selected from the group consisting of: compounds A1.1 to A28.1.

Another embodiment of this invention is directed to a compound of formula (I) selected from the group consisting of: compounds A12 to A22.2, and A24.2 to A282.

Another embodiment of this invention is directed to a compound of formula (I) selected from the group consisting of: compounds 5.1, 8.1, and 11.1.

Another embodiment of this invention is directed to a compound of formula (I) selected from the group consisting of: compounds A1 to A28.

Another embodiment of this invention is directed to compound 5.1.

Another embodiment of this invention is directed to compound 8.1.

Another embodiment of this invention is directed to compound 11.1.

Another embodiment of this invention is directed to compound A1.

Another embodiment of this invention is directed to compound A2.

Another embodiment of this invention is directed to compound A3.

Another embodiment of this invention is directed to compound A4.

Another embodiment of this invention is directed to compound A5.

Another embodiment of this invention is directed to compound A6.

Another embodiment of this invention is directed to compound A7.

Another embodiment of this invention is directed to compound A8.

Another embodiment of this invention is directed to compound A9.

Another embodiment of this invention is directed to compound A10.

Another embodiment of this invention is directed to compound A11.

Another embodiment of this invention is directed to compound A12.

Another embodiment of this invention is directed to compound A13.

Another embodiment of this invention is directed to compound A14.

Another embodiment of this invention is directed to compound A15.

Another embodiment of this invention is directed to compound A16.

Another embodiment of this invention is directed to compound A17.

Another embodiment of this invention is directed to compound A18.

Another embodiment of this invention is directed to compound A19.

Another embodiment of this invention is directed to compound A20.

Another embodiment of this invention is directed to compound A21.

Another embodiment of this invention is directed to compound A22.

Another embodiment of this invention is directed to compound A23.

Another embodiment of this invention is directed to compound A24.

Another embodiment of this invention is directed to compound A25.

Another embodiment of this invention is directed to compound A26.

Another embodiment of this invention is directed to compound A27.

Another embodiment of this invention is directed to compound A28.

In the embodiments below Groups A, B, C, D and E are as defined as follows:
(1) Group A: compounds IA to IE, 1A to 4A, A11.1 to A28.1, A1.2 to A22.2, A24.2 to A28.2, 5.1, 8.1, 11.1, and A1 to A28;
(2) Group B: compounds IA to IE;
(3) Group C: compounds 1A to 4A, A1.1 to A28.1, A1.2 to A22.2, and A24.2 to A28.2;
(4) Group D: compounds 5.1, 8.1, and 11.1; and
(5) Group E: compounds A1 to A28.

Another embodiment of this invention is directed to a compound of formula (I).

Another embodiment of this invention is directed to a pharmaceutically acceptable salt of a compound of formula (I). And in one example the salt is a salt of a compound selected from the group consisting of Group A. And in another example the salt is a salt of a compound selected from the group consisting of Group B. And in another example the salt is a salt of a compound selected from the group consisting of Group C. And in another example the salt is a salt of a compound selected from the group consisting of Group D. And in another example the salt is a salt of a compound selected from the group consisting of Group E.

Another embodiment of this invention is directed to a pharmaceutically acceptable ester of a compound of formula (I). And in one example the ester is an ester of a compound selected from the group consisting of Group A. And in another example the ester is an ester of a compound selected from the group consisting of Group B. And in another example the ester is an ester of a compound selected from the group consisting of Group C. And in another example the ester is an ester of a compound selected from the group consisting of Group D. And in another example the ester is an ester of a compound selected from the group consisting of Group E.

Another embodiment of this invention is directed to a solvate of a compound of formula (I). And in one example the solvate is a solvate of a compound selected from the group consisting of Group A, And in another example the solvate is a solvate of a compound selected from the group consisting of Group B. And in another example the solvate is a solvate of a compound selected from the group consisting of Group C. And in another example the solvate is a solvate of a compound selected from the group consisting of Group D. And in another example the solvate is a solvate of a compound selected from the group consisting of Group E.

Another embodiment of this invention is directed to a compound of formula (I) in isolated form. And in one example the compound of formula (I) is selected from the group consisting of Group A. And in one example the compound of formula (I) is selected from the group consisting of Group D. And in one example the compound of formula (I) is selected from the group consisting of Group E.

Another embodiment of this invention is directed to a compound of formula (I) in pure form. And in one example the compound of formula (I) is selected from the group consisting of Group A. And in one example the compound of formula (I) is selected from the group consisting of Group D. And in one example the compound of formula (I) is selected from the group consisting of Group E.

Another embodiment of this invention is directed to a compound of formula (I) in pure and isolated form. And in one example the compound of formula (I) is selected from the group consisting of Group A. And in one example the compound of formula (I) is selected from the group consisting of Group D. And in one example the compound of formula (I) is selected from the group consisting of Group E.

Another embodiment of this invention is directed to a pharmaceutical composition comprising an effective amount of one or more (e.g., one) compounds of Formula (I), or a pharmaceutically acceptable salt, solvate, or ester thereof, and one or more (e.g., one) pharmaceutically acceptable carriers.

Another embodiment is directed to a pharmaceutical composition comprising an effective amount of one or more (e.g., one) compounds of formula (I) and a pharmaceutically acceptable carrier.

Another embodiment is directed to a pharmaceutical composition comprising an effective amount of a pharmaceutically acceptable salt of one or more (e.g., one) compounds of formula (I) and a pharmaceutically acceptable carrier.

Another embodiment is directed to a pharmaceutical composition comprising an effective amount of a pharmaceutically acceptable ester of one or more (e.g., one) compounds of formula (I) and a pharmaceutically acceptable carrier,

Another embodiment is directed to a pharmaceutical composition comprising an effective amount of a solvate of one or more (e.g., one) compounds of formula (I) and a pharmaceutically acceptable carrier.

Another embodiment of this invention is directed to a pharmaceutical composition comprising an effective amount of one or more (e.g., one) compounds of formula (I), and an effective amount of one or more (e.g., one) other pharmaceutically active ingredients (e.g., drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising a therapeutically effective amount of one or more (e.g. one) compounds of Formula (I), or a pharmaceutically acceptable salt, solvate, or ester thereof, and one or more (e.g., one) pharmaceutically acceptable carriers, and an effective amount of one or more compounds selected from the group consisting of cholinesterase inhibitors, Aβ antibody inhibitors, gamma secretase inhibitors and beta secretase inhibitors.

Another embodiment of this invention is directed to a pharmaceutical composition comprising an effective amount of one or more (e.g., one) compounds of formula (I), and effective amount of one or more BACE inhibitors, and a pharmaceutically acceptable carrier.

Another embodiment of this invention is directed to a pharmaceutical composition comprising an effective amount of one or more (e.g., one) compounds of formula (I), and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds of formula (I).

Another embodiment of this invention is directed to a pharmaceutical composition comprising an effective amount of one or more (e.g., one) compounds of formula (I), and effective amount of one or more muscarinic antagonists (e.g., m₁ agonist or m₂ antagonists), and a pharmaceutically acceptable carrier.

Another embodiment of this invention is directed to a pharmaceutical composition comprising an effective amount of one or more (e.g., one) compounds of formula (I), and effective amount of Exelon (rivastigmine), and a pharmaceutically acceptable carrier.

Another embodiment of this invention is directed to a pharmaceutical composition comprising an effective amount of one or more (e.g., one) compounds of formula (I), and effective amount of Cognex (tacrine), and a pharmaceutically acceptable carrier.

Another embodiment of this invention is directed to a pharmaceutical composition comprising an effective amount of one or more (e.g., one) compounds of formula (I), and effective amount of a Tau kinase inhibitor, and a pharmaceutically acceptable carrier.

Another embodiment of this invention is directed to a pharmaceutical composition comprising an effective amount of one or more (e.g., one) compounds of formula (I), and effective amount of one or more Tau kinase inhibitor (e.g., GSK3beta inhibitor, cdk5 inhibitor, ERK inhibitor), and a pharmaceutically acceptable carrier.

Another embodiment of this invention is directed to a pharmaceutical composition comprising an effective amount of one or more (e.g., one) compounds of formula (I), and effective amount of one anti-Abeta vaccine (active immunization), and a pharmaceutically acceptable carrier.

Another embodiment of this invention is directed to a pharmaceutical composition comprising an effective amount of one or more (e.g., one) compounds of formula (I), and effective amount of one or more APP ligands, and a pharmaceutically acceptable carrier.

Another embodiment of this invention is directed to a pharmaceutical composition comprising an effective amount of one or more (e.g., one) compounds of formula (I), and effective amount of one or more agents that upregulate insulin degrading enzyme and/or neprilysin, and a pharmaceutically acceptable carrier.

Another embodiment of this invention is directed to a pharmaceutical composition comprising an effective amount of one or more (e.g., one) compounds of formula (I), and effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe), and a pharmaceutically acceptable carrier.

Another embodiment of this invention is directed to a pharmaceutical composition comprising an effective amount of one or more (e.g., one) compounds of formula (I), and effective amount of one or more fibrates (for example, clofibrate, Clofibide, Etofibrate, Aluminium Clofibrate), and a pharmaceutically acceptable carrier

Another embodiment of this invention is directed to a pharmaceutical composition comprising an effective amount of one or more (e.g., one) compounds of formula (I), and effective amount of one or more LXR agonists, and a pharmaceutically acceptable carrier.

Another embodiment of this invention is directed to a pharmaceutical composition comprising an effective amount of one or more (e.g., one) compounds of formula (I), and effective amount of one or more LRP mimics, and a pharmaceutically acceptable carrier.

Another embodiment of this invention is directed to a pharmaceutical composition comprising an effective amount of one or more (e.g., one) compounds of formula (I), and effective amount of one or more 5-HT6 receptor antagonists, and a pharmaceutically acceptable carrier.

Another embodiment of this invention is directed to a pharmaceutical composition comprising an effective amount of one or more (e.g., one) compounds of formula (I), and effective amount of one or more nicotinic receptor agonists, and a pharmaceutically acceptable carrier.

Another embodiment of this invention is directed to a pharmaceutical composition comprising an effective amount of one or more (e.g., one) compounds of formula (I), and effective amount of one or more H3 receptor antagonists, and a pharmaceutically acceptable carrier.

Another embodiment of this invention is directed to a pharmaceutical composition comprising an effective amount of one or more (e.g., one) compounds of formula (I), and effective amount of one or more histone deacetylase inhibitors, and a pharmaceutically acceptable carrier.

Another embodiment of this invention is directed to a pharmaceutical composition comprising an effective amount of one or more (e.g., one) compounds of formula (I), and effective amount of one or more hsp90 inhibitors, and a pharmaceutically acceptable carrier.

Another embodiment of this invention is directed to a pharmaceutical composition comprising an effective amount of one or more (e.g., one) compounds of formula (I), and effective amount of one or more m1 muscarinic receptor agonists, and a pharmaceutically acceptable carrier.

Another embodiment of this invention is directed to combinations, i.e., a pharmaceutical composition, comprising a pharmaceutically acceptable carrier, an effective (i.e., therapeutically effective) amount of one or more compounds of formula (I), in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept®brand of donepezil hydrochloride), Aβ antibody inhibitors, gamma secretase inhibitors and beta secretase inhibitors.

Another embodiment of this invention is directed to a pharmaceutical composition comprising an effective amount of one or more (e.g., one) compounds of formula (I), and effective amount of one or more 5-HT6 receptor antagonists mGluR1 or mGluR5 positive allosteric modulators or agonists, and a pharmaceutically acceptable carrier.

Another embodiment of this invention is directed to a pharmaceutical composition comprising an effective amount of one or more (e.g., one) compounds of formula (I), and effective amount of one or more one mGluR2/3 antagonists, and a pharmaceutically acceptable carrier.

Another embodiment of this invention is directed to a pharmaceutical composition comprising an effective amount of one or more (e.g., one) compounds of formula (I), and effective amount of one or more anti-inflammatory agents that can reduce neuroinflammation, and a pharmaceutically acceptable carrier.

Another embodiment of this invention is directed to a pharmaceutical composition comprising an effective amount of one or more (e.g., one) compounds of formula (I), and effective amount of one or more Prostaglandin EP2 receptor antagonists, and a pharmaceutically acceptable carrier.

Another embodiment of this invention is directed to a pharmaceutical composition comprising an effective amount of one or more (e.g., one) compounds of formula (I), and effective amount of one or more PAI-1 inhibitors, and a pharmaceutically acceptable carrier.

Another embodiment of this invention is directed to a pharmaceutical composition comprising an effective amount of one or more (e.g., one) compounds of formula (I), and effective amount of one or more agents that can induce Abeta efflux such as geisoin, and a pharmaceutically acceptable carrier.

Other embodiments of this invention are directed to any one of the above embodiments directed to pharmaceutical compositions wherein the compound of formula (I) is selected from the group consisting of Group A.

Other embodiments of this invention are directed to any one of the above embodiments directed to pharmaceutical compositions wherein the compound of formula (I) is selected from the group consisting of Group B.

Other embodiments of this invention are directed to any one of the above embodiments directed to pharmaceutical compositions wherein the compound of formula (I) is selected from the group consisting of Group C.

Other embodiments of this invention are directed to any one of the above embodiments directed to pharmaceutical compositions wherein the compound of formula (I) is selected from the group consisting of Group D.

Other embodiments of this invention are directed to any one of the above embodiments directed to pharmaceutical compositions wherein the compound of formula (I) is selected from the group consisting of Group E.

The compounds of formula (I) can be useful as gamma secretase modulators and can be useful in the treatment and prevention of diseases such as, for example, central nervous system disorders (such as Alzheimers disease and Downs Syndrome), mild cognitive impairment, glaucoma, cerebral amyloid angiopathy, stroke, dementia, microgliosis, brain inflammation, and olfactory function loss.

Another embodiment of this invention is directed to a method of treating a central nervous system disorder comprising administering a therapeutically effective amount of at least one compound of formula (I) to a patient in need of such treatment.

Another embodiment of this invention is directed to a method of treating a central nervous system disorder comprising administering a therapeutically effective amount of a pharmaceutical composition comprising a therapeutically effective amount of at least one compound of formula (I), or a pharmaceutically acceptable salt, solvate, or ester thereof, and at least one pharmaceutically acceptable carrier.

Another embodiment of this invention is directed to a method of treating a central nervous system disorder comprising administering a therapeutically effective amount of a pharmaceutical composition comprising a therapeutically effective amount of at least one compound of formula (I), or a pharmaceutically acceptable salt, solvate, or ester thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

Thus, another embodiment of this invention is directed to a method for modulating (including inhibiting, antagonizing and the like) gamma-secretase comprising administering an effective (i.e., therapeutically effective) amount of one or more (e.g., one) compounds of formula (I) to a patient in need of such treatment.

Another embodiment of this invention is directed to a method for modulating (including inhibiting, antagonizing and the like) gamma-secretase, comprising administering an effective (i.e., therapeutically effective) amount of a compound of formula (I) to a patient in need of treatment.

Another embodiment of this invention is directed to a method of treating one or more neurodegenerative diseases, comprising administering an effective (i.e., therapeutically effective) amount of one or more (e.g., one) compounds of formula (I) to a patient in need of treatment.

Another embodiment of this invention is directed to a method of treating one or more neurodegenerative diseases, comprising administering an effective (i.e., therapeutically effective) amount of a compound of formula (I) to a patient in need of treatment.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of one or more (e.g., one) compounds of formula (I) to a patient in need of treatment.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of a compound of formula (I) to a patient in need of treatment.

Another embodiment of this invention is directed to a method of treating Alzheimer's disease, comprising administering an effective (i.e., therapeutically effective) amount of one or more (e.g., one) compounds of formula (I) to a patient in need of treatment.

Another embodiment of this invention is directed to a method of treating Alzheimer's disease, comprising administering an effective (i.e., therapeutically effective) amount of a compound of formula (I) to a patient in need of treatment.

Another embodiment of this invention is directed to a method of treating Alzheimer's disease, comprising administering an effective (i.e., therapeutically effective) amount of a compound of formula (I) to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of one or more (e.g., one) compounds of formula (I) to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of a compound of formula (I) to a patient in need of treatment.

Another embodiment of this invention is directed to a method of treating mild cognitive impairment, comprising administering an effective amount of one or more (e.g., one) compounds of formula (I) to a patient in need of treatment.

Another embodiment of this invention is directed to a method of treating glaucoma, comprising administering an effective amount of one or more (e.g., one) compounds of formula (I) to a patient in need of treatment.

Another embodiment of this invention is directed to a method of treating cerebral amyloid angiopathy, comprising administering an effective amount of one or more (e.g., one) compounds of formula (I) to a patient in need of treatment.

Another embodiment of this invention is directed to a method of treating stroke, comprising administering an effective amount of one or more (e.g., one) compounds of formula (I) to a patient in need of treatment.

Another embodiment of this invention is directed to a method of treating dementia, comprising administering an effective amount of one or more (e.g., one) compounds of formula (I) to a patient in need of treatment.

Another embodiment of this invention is directed to a method of treating microgliosis, comprising administering an effective amount of one or more (e.g., one) compounds of formula (I) to a patient in need of treatment.

Another embodiment of this invention is directed to a method of treating brain inflammation, comprising administering an effective amount of one or more (e.g., one) compounds of formula (I) to a patient in need of treatment.

Another embodiment of this invention is directed to a method of treating olfactory function loss, comprising administering an effective amount of one or more (e.g., one) compounds of formula (I) to a patient in need of treatment.

Another embodiment of this invention is directed to a method of treating Downs syndrome, comprising administering an effective amount of one or more (e.g., one) compounds of formula (I) to a patient in need of treatment.

Another embodiment of this invention is directed to a method of treating Downs syndrome, comprising administering an effective amount of a compound of formula (I) to a patient in need of treatment.

Other embodiments of this invention are directed to any one of the above embodiments directed to methods of treating wherein the compound of formula (I) is selected from the group consisting of Group A.

Other embodiments of this invention are directed to any one of the above embodiments directed to methods of treating wherein the compound of formula (I) is selected from the group consisting of Group B.

Other embodiments of this invention are directed to any one of the above embodiments directed to methods of treating wherein the compound of formula (I) is selected from the group consisting of Group C.

Other embodiments of this invention are directed to any one of the above embodiments directed to methods of treating wherein the compound of formula (I) is selected from the group consisting of Group D.

Other embodiments of this invention are directed to any one of the above embodiments directed to methods of treating wherein the compound of formula (I) is selected from the group consisting of Group E.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (I) and the administration of one or more (e.g., one) other pharmaceutical active ingredients (e.g., drugs). The compounds of formula (I) ad the other drugs can be administered separately (i.e., each is in its own separate dosage form), or the compounds of formula (I) can be combined with the other drugs in the same dosage form.

Thus, other embodiments of this invention are directed to any one of the methods of treatment, or methods of inhibiting, described herein, wherein an effective amount of the compound of formula (I) is used in combination with an effective amount of one or more other pharmaceutically active ingredients (e.g., drugs). The other pharmaceutically active ingredients (i.e., drugs) are selected from the group consisting of: BACE inhibitors (beta secretase inhibitors), muscarinic antagonists (e.g., m₁ agonists or m₂ antagonists), cholinesterase inhibitors (e.g., acetyl- and/or butyrylchtolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; m1 muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Other embodiments of this invention are directed to any one of the methods of treatment, or methods of inhibiting, described herein, wherein the compound of formula (I) is used in combination with an effective amount of one or more other pharmaceutically active ingredients selected from the group consisting of: BACE inhibitors (beta secretase inhibitors), muscarinic antagonists (e.g., m₁ agonist or m₂ antagonists) cholinesterase inhibitors (e.g. acetyl- and/or butrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors (e.g., ezetimibe).

Another embodiment of this invention is directed to a method of treating Alzheimer's disease, comprising administering an effective (i.e., therapeutically effective) amount of one or more (e.g., one) compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective (i.e., therapeutically effective) amount of a compound of formula (I), in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective (i.e., therapeutically effective) amount of one or more (e.g., one) compounds of formula (I), in combination with an effective (i.e., therapeutically effective) amount of one or more compounds selected from the group consisting of Aβ antibody inhibitors, gamma secretase inhibitors and beta secretase inhibitors.

Another embodiment of this invention is directed to a method of treating Alzheimer's disease, comprising administering an effective (i.e., therapeutically effective) amount of one or more (e.g., one) compounds of formula (I), in combination with an effective (i.e., therapeutically effective) amount of one or more BACE inhibitors.

Another embodiment of this invention is directed to a method of treating Alzheimer's disease, comprising administering an effective amount of one or more compounds of formula (I), in combination with an effective amount of Exelon (rivastigmine).

Another embodiment of this invention is directed to a method of treating Alzheimer's disease, comprising administering an effective amount of one or more compounds of formula (I), in combination with an effective amount of Cognex (tacrine).

Another embodiment of this invention is directed to a method of treating Alzheimer's disease, comprising administering an effective amount of one or more compounds of formula (I), in combination with an effective amount of a Tau kinase inhibitor.

Another embodiment of this invention is directed to a method of treating Alzheimer's disease, comprising administering an effective amount of one or more compounds of formula (I), in combination with an effective amount of one or more Tau kinase inhibitor (e.g., GSK3beta inhibitor, cdk5 inhibitor, ERK inhibitor).

This invention also provides a method of treating Alzheimer's disease, comprising administering an effective amount of one or more compounds of formula (I), in combination with an effective amount of one anti-Abeta vaccination (active immunization).

Another embodiment of this invention is directed to a method of treating Alzheimer's disease, comprising administering an effective amount of one or more compounds of formula (I), in combination with an effective amount of one or more APP ligands.

Another embodiment of this invention is directed to a method of treating Alzheimer's disease, comprising administering an effective amount of one or more compounds of formula (I), in combination with an effective amount of one or more agents that upregulate insulin degrading enzyme and/or neprilysin.

Another embodiment of this invention is directed to a method of treating Alzheimer's disease, comprising administering an effective amount of one or more compounds of formula (I), in combination with an effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe).

This invention also provides a method of treating Alzheimer's disease, comprising administering an effective amount of one or more compounds of formula (I), in combination with an effective amount of one or more fibrates (for example, clofibrate, Clofibride, Etofibrate, Aluminium Clofibrate).

Another embodiment of this invention is directed to a method of treating Alzheimer's disease, comprising administering an effective amount of one or more compounds of formula (I), in combination with an effective amount of one or more LXR agonists.

Another embodiment of this invention is directed to a method of treating Alzheimer's disease, comprising administering an effective amount of one or more compounds of formula (I), in combination with an effective amount of one or more LRP mimics.

Another embodiment of this invention is directed to a method of treating Alzheimer's disease, comprising administering an effective amount of one or more compounds of formula (I), in combination with an effective amount of one or more 5-HT6 receptor antagonists.

Another embodiment of this invention is directed to a method of treating Alzheimer's disease, comprising administering an effective amount of one or more compounds of formula (I), in combination with an effective amount of one or more nicotinic receptor agonists.

Another embodiment of this invention is directed to a method of treating Alzheimer's disease, comprising administering an effective amount of one or more compounds of formula (I), in combination with an effective amount of one or more H3 receptor antagonists.

This invention also provides a method of treating Alzheimer's disease, comprising administering an effective amount of one or more compounds of formula (I), in combination with an effective amount of one or more histone deacetylase inhibitors.

Another embodiment of this invention is directed to a method of treating Alzheimer's disease, comprising administering an effective amount of one or more compounds of formula (I), in combination with an effective amount of one or more hsp90 inhibitor.

Another embodiment of this invention is directed to a method of treating Alzheimer's disease, comprising administering an effective amount of one or more compounds of formula (I), in combination with an effective amount of one or more m1 muscarinic receptor agonists.

Another embodiment of this invention is directed to a method of treating Alzheimer's disease, comprising administering an effective amount of one or more compounds of formula (I), in combination with an effective amount of one or more 5-HT6 receptor antagonists mGluR1 or mGluR5 positive allosteric modulators or agonists.

Another embodiment of this invention is directed to a method of treating Alzheimer's disease, comprising administering an effective amount of one or more compounds of formula (I), in combination with an effective amount of one or more mGluR2/3 antagonists.

Another embodiment of this invention is directed to a method of treating Alzheimer's disease, comprising administering an effective amount of one or more compounds of formula (I), in combination with an effective amount of one or more anti-inflammatory agents that can reduce neuroinflammation.

Another embodiment of this invention is directed to a method of treating Alzheimer's disease, comprising administering an effective amount of one or more compounds of formula (I), in combination with an effective amount of one or more Prostaglandin EP2 receptor antagonists.

Another embodiment of this invention is directed to a method of treating Alzheimer's disease, comprising administering an effective amount of one or more compounds of formula (I), in combination with an effective amount of one or more PAI-1 inhibitors.

Another embodiment of this invention is directed to a method of treating Alzheimer's disease, comprising administering an effective amount of one or more compounds of formula (I), in combination with an effective amount of one or more agents that can induce Abeta efflux such as gelsolin.

Another embodiment of this invention is directed to a method of treating Downs syndrome, comprising administering an effective (i.e., therapeutically effective) amount of one or more (e.g., one) compounds of formula (I) to a patient in need of treatment.

Another embodiment of this invention is directed to a method of treating Downs syndrome, comprising administering an effective (i.e., therapeutically effective) amount of a compound of formula (I) to a patient in need of treatment.

Another embodiment of this invention is directed to a method of treating Downs syndrome, comprising administering an effective (i.e., therapeutically effective) amount of one or more (e.g., one) compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Downs syndrome, comprising administering an effective (i.e., therapeutically effective) amount of a compound of formula (I), in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to combinations (i.e., pharmaceutical compositions) comprising an effective (i.e., therapeutically effective) amount of one or more (e.g., one) compounds of formula (I), in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors. The pharmaceutical compositions also comprise a pharmaceutically acceptable carrier.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (f) is selected from the group consisting of Group A.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group B.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group C.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group D.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group E.

Another embodiment of this invention is directed to a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase, or (e) mild cognitive impairment, or (f) glaucoma, or (g) cerebral amyloid angiopathy, or (h) stroke, or (i) dementia, or (j) microgliosis, or (k) brain inflammation, or (l) olfactory function loss.

Another embodiment of this inventions directed to a kit comprising, in separate containers, in a single package, pharmaceutical compositions for use in combination, wherein one container comprises an effective amount of one or more (e.g., one) compounds of formula (I) in a pharmaceutically acceptable carrier, and another container (i.e., a second container) comprises an effective amount of another pharmaceutically active ingredient (as described above), the combined quantities of the compounds of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Another embodiment of this invention is directed to a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Other embodiments of this invention are directed to any one of the above embodiments directed to kits wherein the compound of formula (I) is selected from the group consisting of Group A.

Other embodiments of this invention are directed to any one of the above embodiments directed to kits wherein the compound of formula (I) is selected from the group consisting of Group B.

Other embodiments of this invention are directed to any one of the above embodiments directed to kits wherein the compound of formula (I) is selected from the group consisting of Group C.

Other embodiments of this invention are directed to any one of the above embodiments directed to kits wherein the compound of formula (I) is selected from the group consisting of Group D.

Other embodiments of this invention are directed to any one of the above embodiments directed to kits wherein the compound of formula (I) is selected from the group consisting of Group E.

Examples of cholinesterase inhibitors are tacrine, donepezil, rivastigmine, galantamine, pyridostigmine and neostigmine, with tacrine, donepezil, rivastigmine and galantamine being preferred.

Examples of agonist are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US2005/0119227 published Jun. 2, 2005 (see also WO2005/016876 published 02/24/2005), US2005/0043290 published 20/24/2005 (see also WO2005/014540 published 02/17/2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published 02/23/2006 (see also WO2006/014762 published Feb. 9, 2006), WO2006/014944 published Feb. 9, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO02006/138217 published Dec. 28, 2006 (see also US2006/0287294 published Dec. 21, 2006), US2007/0099898 published May 3, 200 (see also WO2007/050721 published May 3, 2007), WO2007/053506 published May 10, 2007 (see also US2007/099875 published May 3, 2007), U.S. application Ser. No. 11/759,336 filed Jun. 7, 2007, U.S. Application Ser. No. 60/874,362 filed Dec. 12, 2006, and U.S. Application Ser. No. 60/874,419 filed Dec. 12, 2006, the disclosures of each being incorporated incorporated herein by reference thereto.

As used above, and throughout this disclosure, the following terms, unless otherwise indicated, shall be understood to have the following meanings:

“Patient” includes both human and animals.

“Mammal” means humans and other mammalian animals.

“One or more” means that there is at least one and there can be more than one, and examples include 1, 2 or 3, or 1 and 2, or 1.

“At least one” means there is at least one and there can be more than one, and examples include 1, 2 or 3, or 1 and 2, or 1.

It is noted that the carbons of formula (I) and other formulas herein may be replaced with 1 to 3 silicon atoms so long as all valency requirements are satisfied.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl, aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazoyl, thienopyridyl, quinazolinyl, thienopyrimidyl, pyrrolopyridyl, imidazopyridyl, isoquinolinyl, benzoazaindoyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms.

Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyl include cyclopentenylmethyl, cyclohexenylmethyl and the like.

“Halogen” means fluorine, chlorine, bromine, or iodine. Preferred are fluorine, chlorine and bromine. “Halo” refers to fluoro, chloro, bromo or iodo.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂ NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

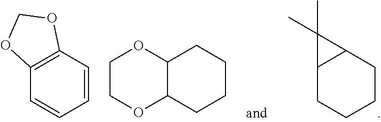

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” (or heterocycloalkyl) means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” may also mean a single moiety (e.g., carbonyl) which simultaneously replaces two available hydrogens on the same carbon atom on a ring system. Example of such moiety is pyrrolidone:

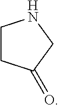

“Heterocyclylalkyl” (or heterocycloalkylalkyl) means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” (or heterocycloalkenyl) means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazoinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a single moiety (e.g., carbonyl) which simultaneously replaces two available hydrogens on the same carbon atom on a ring system. Example of such moiety is pyrrolidinone:

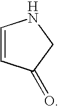

“Heterocyclenylalkyl” (or heterocycloalkenylalkyl) means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

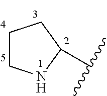

there is no —OH attached directly to carbons marked 2 and 5.

It should also be noted that tautomeric forms such as, for example, the moieties:

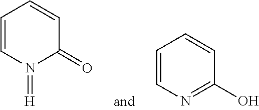

are considered equivalent in certain embodiments of this invention.

“Alkynylalkyl” means an alkynyl-alkyl-group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylethyl.

“Heteroaralkyl” means a heteroaryl-alkyl-group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl-group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

By “stable compound’ or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula (I), its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as 3-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₄)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3, 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula (I) can form salts which are also within the scope of this invention. Reference to a compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula (I) may be formed, for example, by reacting a compound of Formula (I) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxy ic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄ alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of Formula (I), and salts, solvates, esters and prodrugs thereof, may exist in their tautomeric form (for example, as an amide, enol, keto or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, a keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formula (I), and of the salts, solvates, esters and prodrugs of the compounds of Formula (I), are intended to be included in the present invention.

The compounds according to the invention can have pharmacological properties; in particular, the compounds of Formula (I) can be modulators of gamma secretase (including inhibitors, antagonists and the like).

More specifically, the compounds of Formula (I) can be useful in the treatment of a variety of disorders of the central nervous system including, for example, including, but not limited to, Alzheimer's disease, AIDS-related dementia, Parkinson's disease, amyotrophic lateral sclerosis, retinitis pigmentosa, spinal muscular atrophy and cerebellar degeneration and the like.

Another aspect of this invention is a method of treating a mammal (e.g., human) having a disease or condition of the central nervous system by administering a therapeutically effective amount of at least one compound of Formula (I), or a pharmaceutically acceptable salt, solvate, ester or prodrug of said compound to the mammal.

A preferred dosage is about 0.001 to 500 mg/kg of body weight/day of the compound of Formula (I). An especially preferred dosage is about 0.01 to 25 mg/kg of body weight/day of a compound of Formula (I), or a pharmaceutically acceptable salt or solvate of said compound.

The compounds of this invention may also be useful in combination (administered together or sequentially) with one or more additional agents listed above.

The compounds of this invention may also be useful in combination (administered together or sequentially) with one or more compounds selected from the group consisting of Aβ antibody inhibitors, gamma secretase inhibitors and beta secretase inhibitors.

If formulated as a fixed dose, such combination products employ the compounds of this invention within the dosage range described herein and the other pharmaceutically active agent or treatment within its dosage range.

Accordingly, in an aspect, this invention includes combinations comprising an amount of at least one compound of Formula (I), or a pharmaceutically acceptable salt, solvate, ester or prodrug thereof, and an amount of one or more additional agents listed above wherein the amounts of the compounds/treatments result in desired therapeutic effect.

The pharmacological properties of the compounds of this invention may be confirmed by a number of pharmacological assays. Certain assays are exemplified later in this document.

This invention is also directed to pharmaceutical compositions which comprise at least one compound of Formula (I), or a pharmaceutically acceptable salt, solvate, ester or prodrug of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula (I), or a pharmaceutically acceptable salt, solvate, ester or prodrug of said compound and a pharmaceutically acceptable carrier, vehicle or diluent.

Yet another aspect of this invention is a kit comprising an amount of at least one compound of Formula (I), or a pharmaceutically acceptable salt, solvate, ester or prodrug of said compound and an amount of at least one additional agent listed above, wherein the amounts of the two or more ingredients result in desired therapeutic effect.

The invention disclosed herein is exemplified by the following illustrative processes which should not be construed to limit the scope of the invention. Alternative mechanistic pathways and analogous structures will be apparent to those skilled in the art.

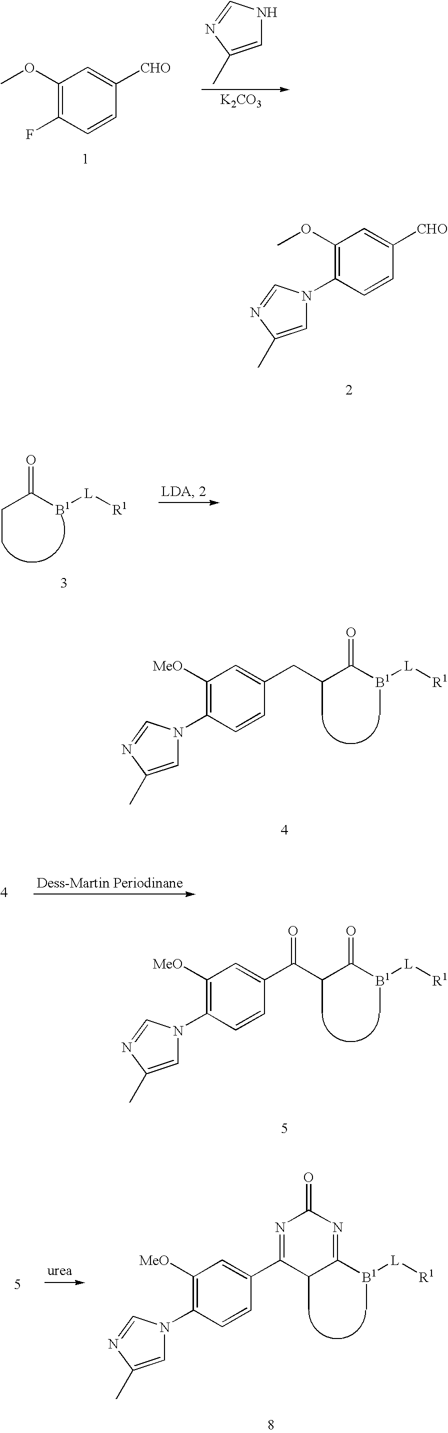

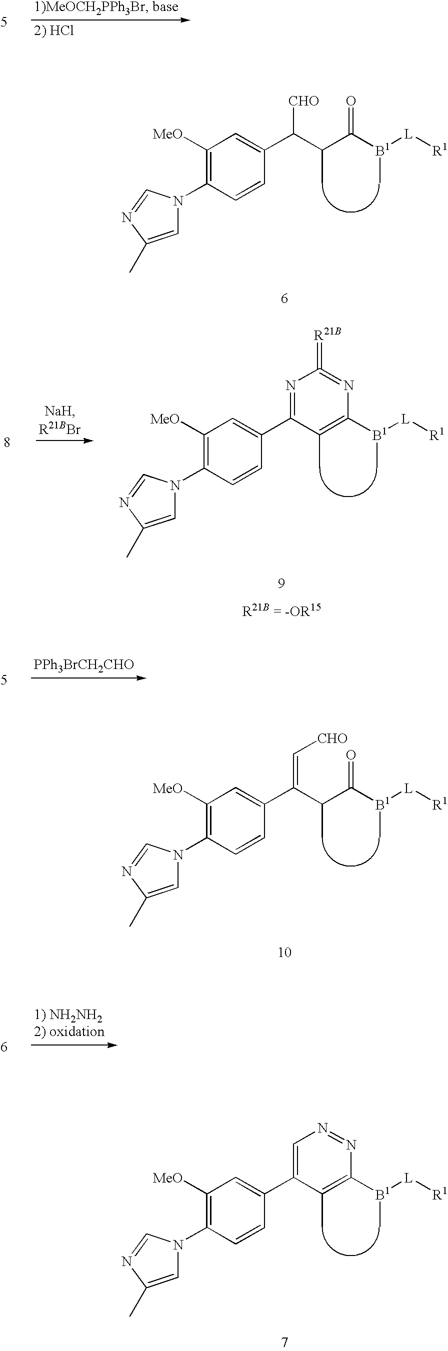

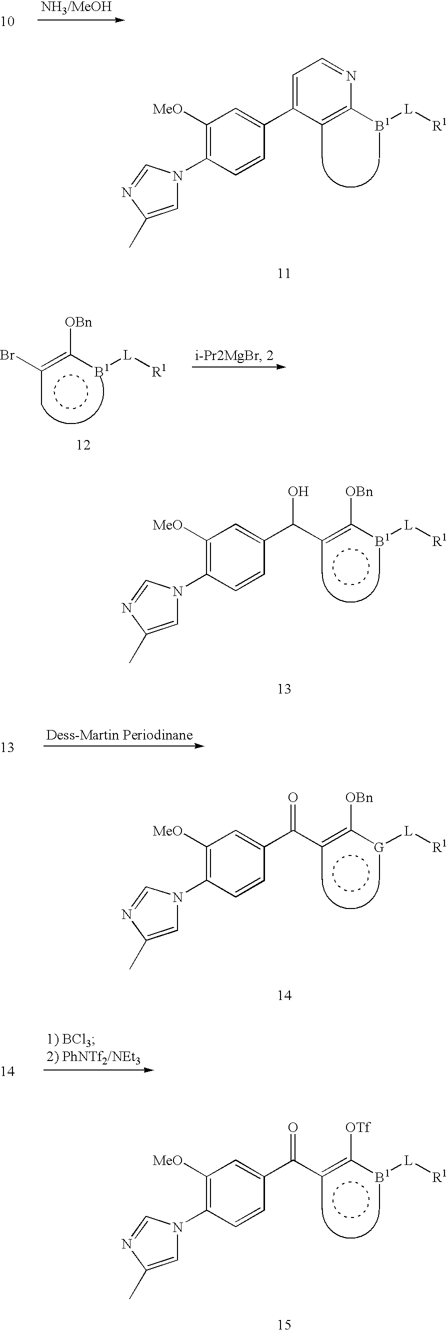

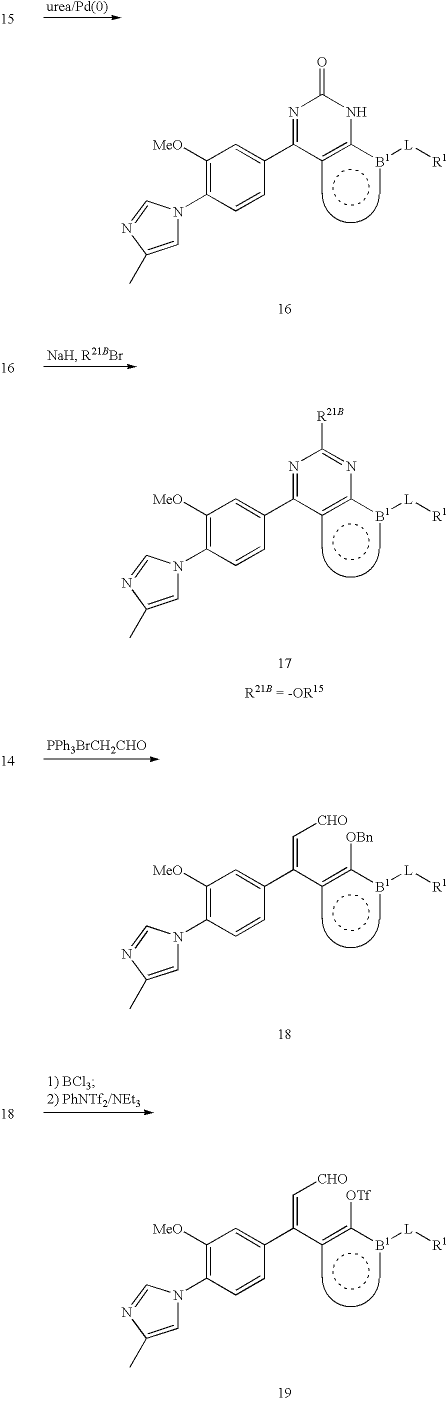

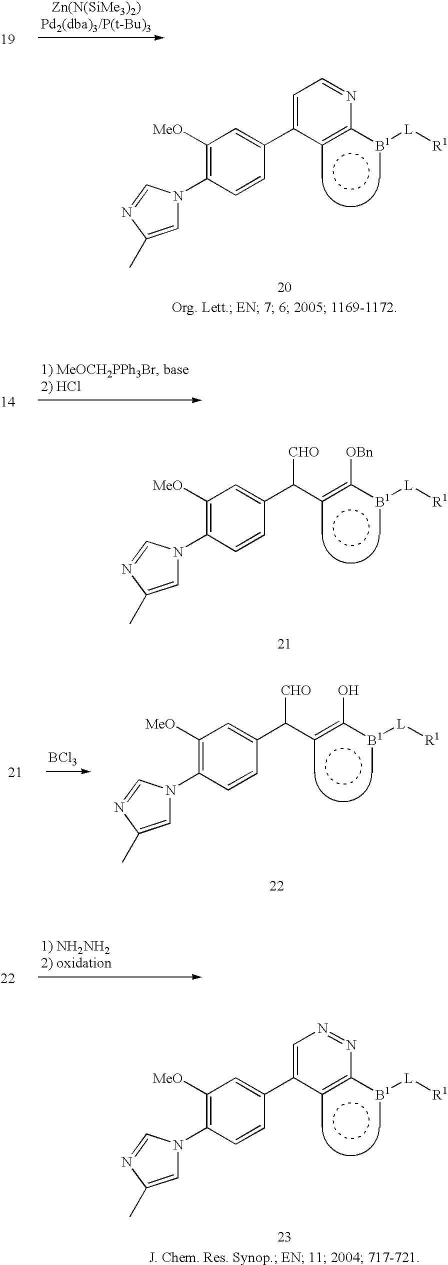

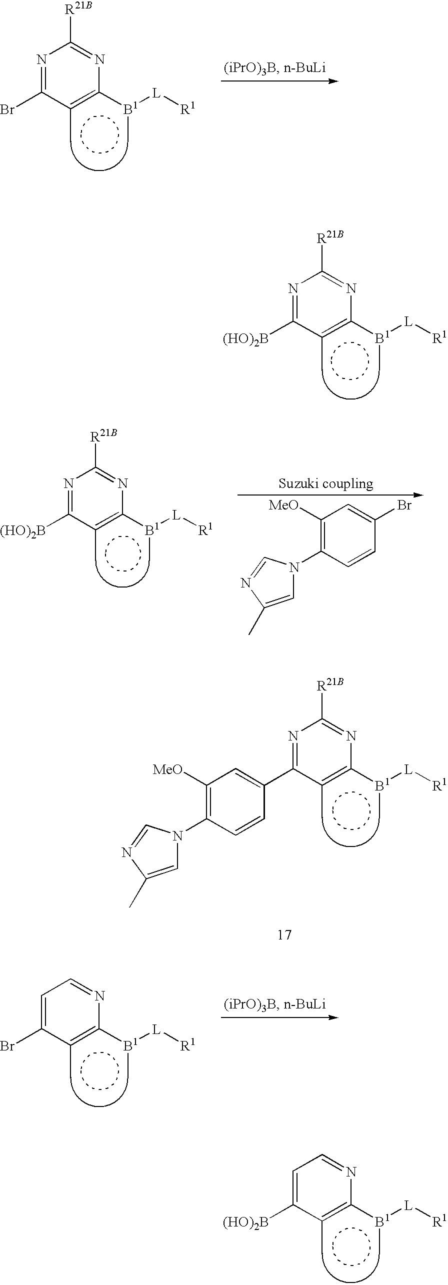

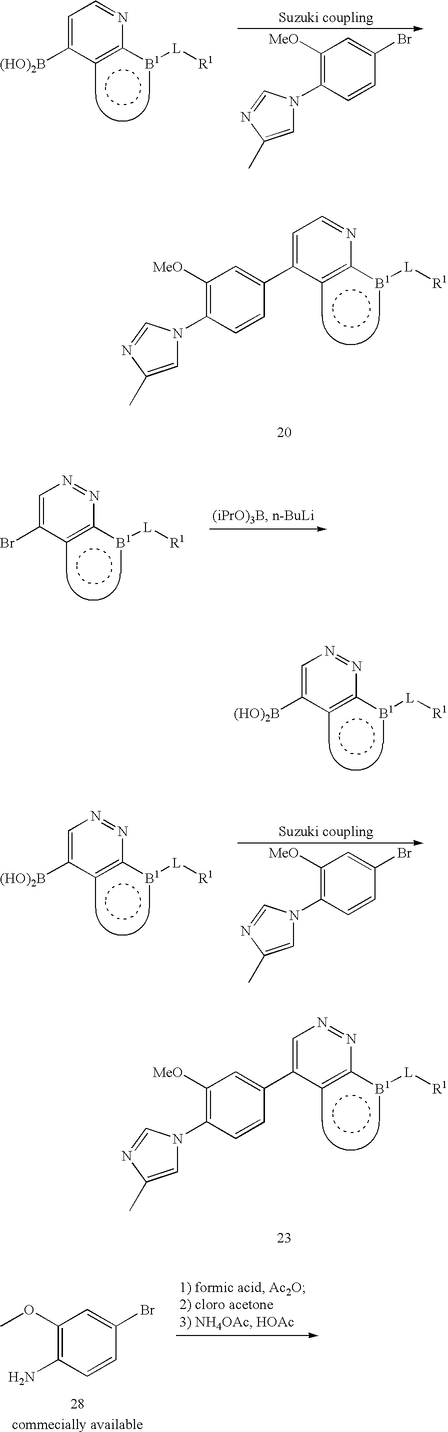

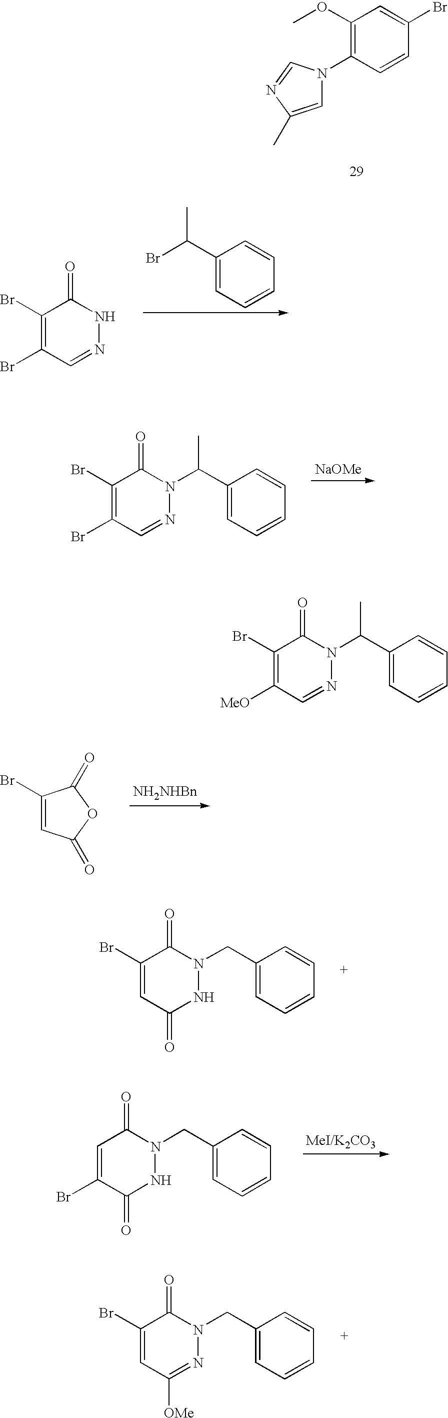

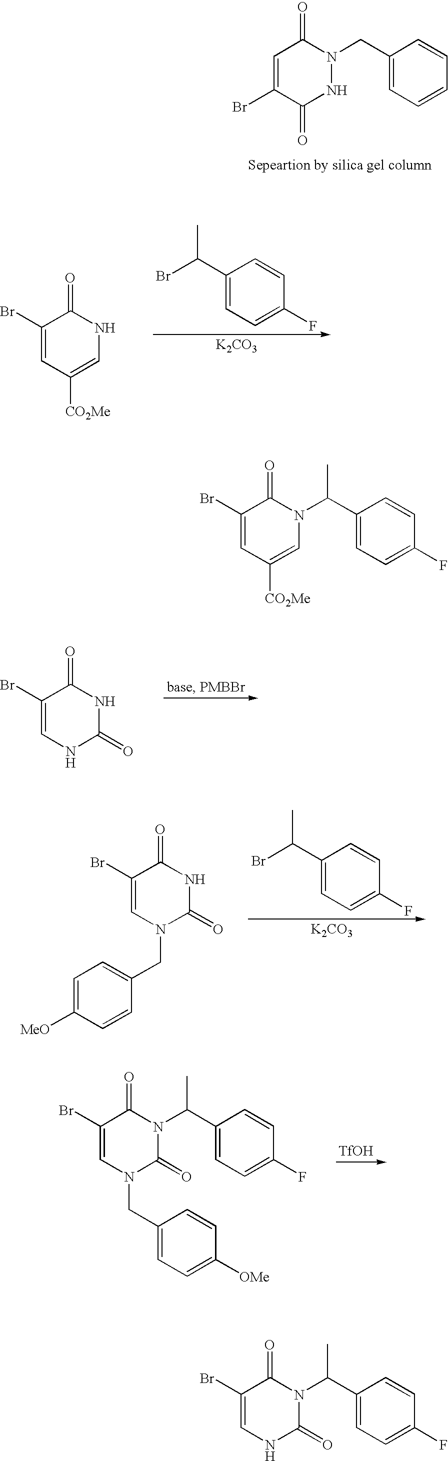

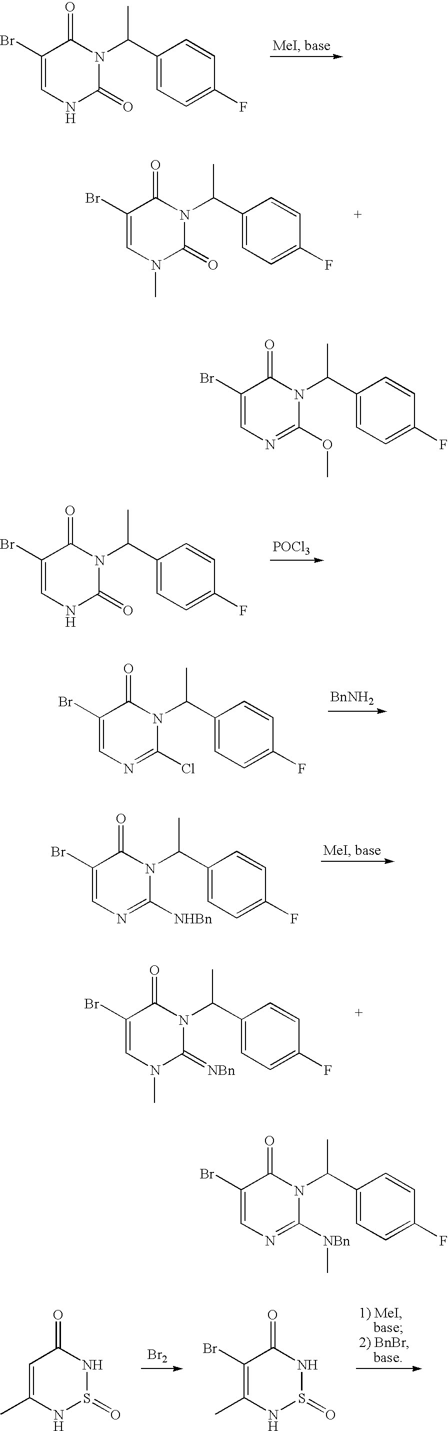

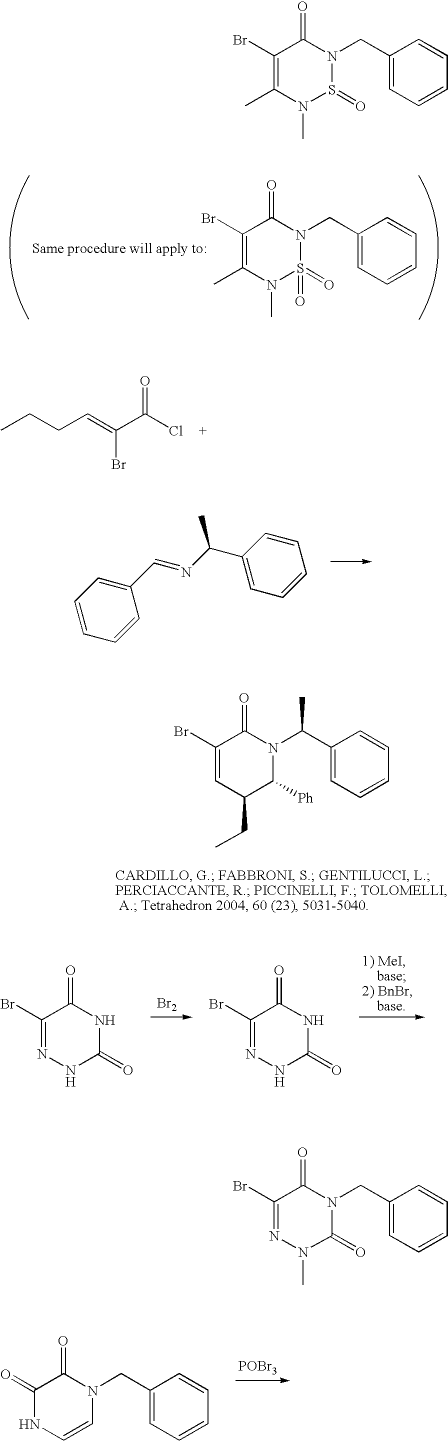

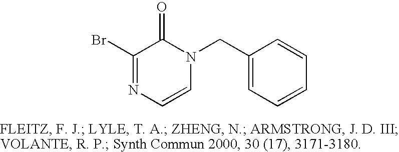

### Example 1

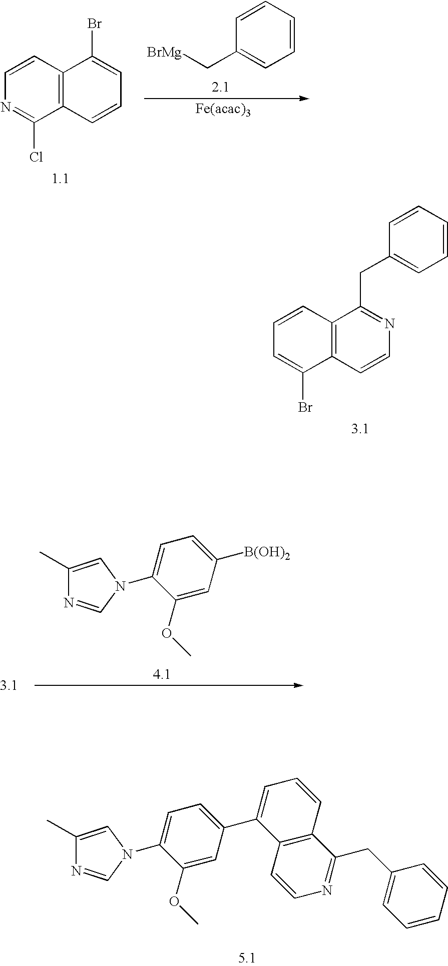

### Step 1

If one were to react Compound 1.1 under N₂ in THF and 5% Fe(acac)₃ with 1 eq of benzylmagnesium bromide in ether at −30 C then one would obtain compound 3.1 after workup.

### Step 2

If one were to mix Compound 3.1 with compound 4.1 (1 eq), K2CO3 (3 eq) and 5% Palladium tetrakistriphenylphosphine in DMF/H2O (99.5/0.5 v/v) and heat the solution to 100° C. under microwave one would obtain compound 5.1 after purification.

### Example 2

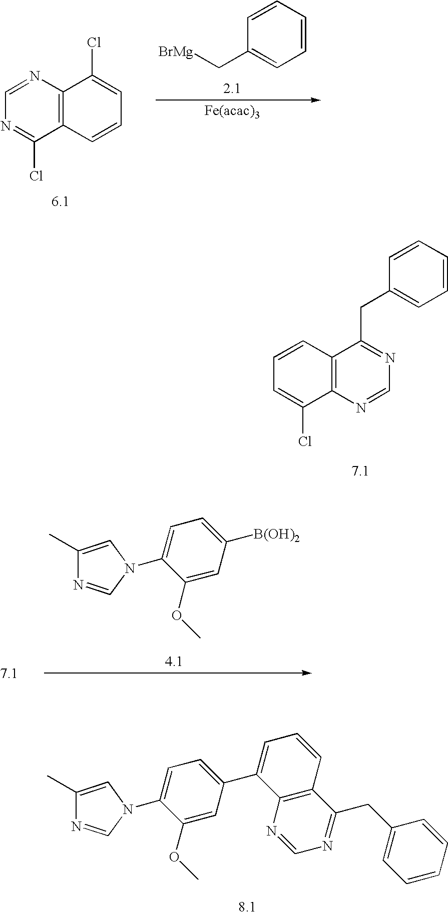

### Step 1

If one were to react Compound 6.1 under N₂ in THF and 5% Fe(acac)₃ with 1 eq of benzylmagnesium bromide in ether at −30° C. one would obtain compound 7.1 after workup.

### Step 2

If one were to mix Compound 4.1 with compound 7.1 (1 eq), K2CO3 (3 eq) and 5% Palladium tetrakistriphenylphosphine in DMF/H2O (99.5/0.5 v/v) and heat the solution to 100° C. under microwave one would obtain compound 8.1 after purification.

### Example 3

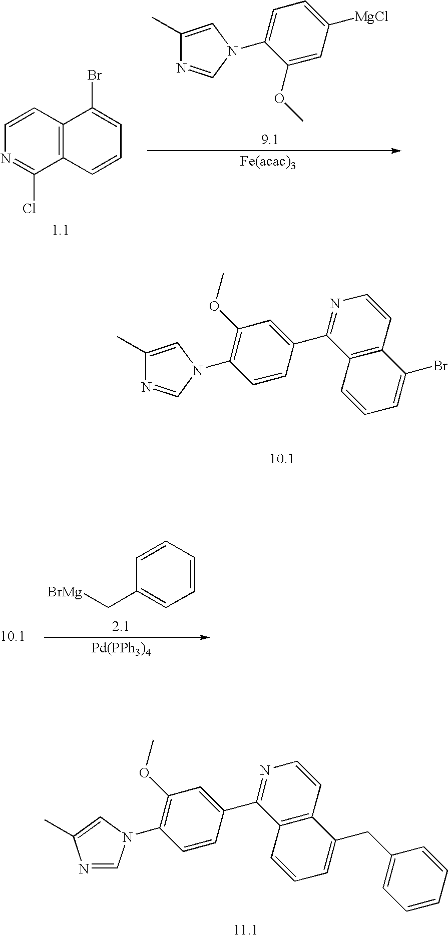

### Step 1

If one were to react Compound 1.1 under N₂ in THF and 5% Fe(acac)₃ with 1 eq of 9.1 (obtain from halogen/metal exchange) in ether at −30 C one would obtain compound 10.1 after workup.

### Step 2

If one would mix Compound 10.1 with tetrakistriphenylphosphine in ether before compound 2.1 (1 eq) would be added one would obtain compound 11.1 after purification.

### Assay:

Secretase Reaction and Aβ Analysis in Whole Cells: HEK293 cells overexpressing APP with Swedish and London mutations is treated with the specified compounds for 5 hour at 37° C. in 100 ml of DMEM medium containing 10% fetal bovine serum. At the end of the incubation, total Aβ, Aβ40 and Aβ42 is measured using electrochemiluminescence (ECL) based sandwich immunoassays. Total Aβ is determined using a pair of antibodies TAG-WO2 and biotin-4G8, Aβ40 is identified with antibody pairs TAG-G2-10 and biotin-4G8, while Aβ342 is identified with TAG-G2-11 and biotin-4G8. The ECL signal is measured using Sector Imager 2400 (Meso Scale Discovery).

MS Analysis of Aβ Profile: Aβ profile in conditioned media is determined using surface enhanced laser desorption/ionization (SELDI) mass spectrometry. Conditioned media is incubated with antibody WO2 coated PS20 ProteinChip array. Mass spectra of Aβcaptured on the array is read on SELDI ProteinChip Reader (Bio-Rad) according to manufacturer's instructions.

CSF Aβ Analysis: Aβ in rat CSF is determined using MSD technology as described above. Aβ40 is measured using antibody pair Tag-G2-10 and biotin-4G8, while Aβ42 is measured using Tag-anti Aβ42 (Meso Scale Discovery) and biotin-4G8. The ECL signal is measured using Sector Imager 2400 (Meso Scale Discovery).

Matrix-assisted laser desorption/ionization mass spectrometric (MALDI MS) analysis of Aβ is performed on a Voyager-DE STR mass spectrometer (ABI, Framingham, Mass.). The instrument is equipped with a pulsed nitrogen laser (337 nm). Mass spectra is acquired in the linear mode with an acceleration voltage of 20 kV. Each spectrum presented in this work represents an average of 256 laser shots. To prepare the sample-matrix solution, 1 μL of immunoprecipitated Aβ sample is mixed with 3 μL of saturated α-cyano-4-hydroxycinnamic acid solution in 0.1% TFA/acetonitrile. The sample-matrix solution is then applied to the sample plate and dried at ambient temperature prior to mass spectrometric analysis. All the spectra are externally calibrated with a mixture of bovine insulin and ACTH (18-39 clip).

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1-55. (canceled)

56. A compound of the formula (I): 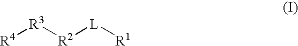
or a pharmaceutically acceptable salt, solvate, ester or prodrug thereof, wherein:
R1, R2, R3, R4 and L are each independently selected;
R1 is selected from the group consisting of: alkyl, alkenyl, alkynyl, cycloalkyl, heterocyclyl, cycloalkenyl, aryl, heteroaryl, heterocyclenyl, fused cycloalkylaryl, fused heterocycloalkylaryl-, fused cycloalkylheteroaryl-, fused heterocycloalkylheteroaryl-, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, and fused heterocycloalkylheteroarylalkyl-, wherein each of said: alkyl, alkenyl, alkynyl, cycloalkyl, heterocyclyl, cycloalkenyl, aryl, heteroaryl, heterocyclenyl fused cycloalkylaryl, fused heterocycloalkylaryl-, fused cycloalkylheteroaryl-, fused heterocycloalkylheteroaryl-, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, and fused heterocycloalkylheteroarylalkyl-R1 groups is optionally substituted with 1-5 independently selected R21 groups;
L is selected from the group consisting of: L is a direct bond, —O—, —N(R5)-, —C(R6)(R7)-, —(C═O)—, —(C═NR21A)-, —S—, —S(O)—, and —S(O)2—;
R2 is the fused bicyclic ring: 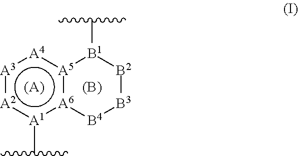
wherein:
(1) Ring (A) is a six membered heteroaryl ring comprising atoms A1 to A6, wherein:
(a) A1 is C,
(b) A5 and A6 are C,
(b) A2, A3 and A4 are each independently selected from the group consisting of: N and C, and wherein each substitutable C is optionally substituted with one R21B group and each R21B for each C is independently selected, and
(c) provided that at least one of A2 to A4 is N, and provided that the total number of nitrogens in Ring (A) is 1 to 3,
(2) Ring (B) (which comprises atoms A5, A6, and B1 to B4) is a cycloalkyl, cycloalkenyl, heterocycloalkyl, heterocycloalkenyl, heteroaryl or phenyl ring, and
(a) A5 and A6 are as defined for Ring (A) above,
(b) in said phenyl Ring (B):
(i) B1 to B4 are C, and
(ii) B2, B3, and B4 are each optionally substituted with one R21B group (and the substitution on each carbon is independent of the substitutions on the remaining carbons),
(c) in said cycloalkyl Ring (B):
(i) B1 is C,
(ii) B2, B3, and B4 are each independently selected from the group consisting of: C, —(C═O)— and —(C═NR21A)-, provided that there are only 0 to 2 moieties selected from the group consisting of —(C═O)— and —(C═NR21A)-, and
(iii) each substitutable B1 to B4 C is optionally substituted with 1 or 2 independently selected R21B groups (and the substitution on each carbon is independent of the substitutions on the remaining carbons),
(d) in said cycloalkenyl Ring (B):
(i) B1 is C,
(ii) B2, B3, and B4 are each independently selected from the group consisting of: C, —(C═O)— and —(C═NR21A)-, provided that there are only 0 to 2 moieties selected from the group consisting of —(C═O)— and —(C═NR21A)-,
(iii) each substitutable B1 to B4 C is optionally substituted with 1 or 2 independently selected R21B groups (and the substitution on each carbon is independent of the substitutions on the remaining carbons), and
(iv) said cycloalkenyl Ring (B) comprises one or two double bonds,
(e) in said heterocycloalkyl Ring (B):
(i) B1 is selected from the group consisting of N and C,
(ii) B2, B3 and B4 are each independently selected from the group consisting of: N, C, —(C═O)—, —(C═NR21A)-, O, S, S(O), and S(O)2, and provided that there are no —O—O— bonds, no —O—S— bonds, no O—S(O) bonds, no —O—S(O)2 bonds, and no —N—S— bonds in the ring, and provided that the ring does not comprise three adjacent nitrogen atoms,
(iii) at least one of B1 to B4 is a heteroatom, and provided that when B1 is a heteroatom said heteroatom is N, and the heteroatoms for B2 to B4 are selected from the group consisting of: N, O, S, S(O), and S(O)2, and
(iv) the total number of heteroatoms in said heterocycloalkyl Ring (B) is 1 to 4, and
(v) each substitutable B1 to B4 C is optionally substituted with 1 or 2 independently selected R21B groups (and the substitution on each carbon is independent of the substitutions on the remaining carbons), and
(vi) each substitutable B2 to B4 N is optionally substituted with one R21A group and each R21A for each N is independently selected,
(f) in said heterocycloalkenyl Ring (B):
(i) B1 is selected from the group consisting of N and C,
(ii) B2, B3 and B4 are each independently selected from the group consisting of: N, C, —(C═O)—, —(C═NR21A)-, O, S, S(O), and S(O)2, and provided that there are no —O—O— bonds, no —O—S— bonds, no O—S(O) bonds, no —O—S(O)2 bonds, and no —N—S— bonds in the ring, and provided that the ring does not comprise three adjacent nitrogen atoms,
(iii) at least one of B1 to B4 is a heteroatom, provided that when B1 is a heteroatom said heteroatom is N, and the heteroatoms for B2 to B4 are selected from the group consisting of: N, O, S, S(O), and S(O)2, and
(iv) the total number of heteroatoms in said heterocycloalkenyl Ring (B) is 1 to 4, and
(v) each substitutable B1 to B4 C is optionally substituted with 1 or 2 independently selected R21B groups (and the substitution on each carbon is independent of the substitutions on the remaining carbons),
(vi) each substitutable B2 to B4 N is optionally substituted with one R21A group and each R21A for each N is independently selected, and
(vii) said heterocycloalkenyl Ring (B) comprises one or two double bonds; and
(g) in said heteroaryl Ring (B):
(i) B1 is C,
(ii) B2 to B4 are each independently selected from the group consisting of C and N,
(iii) at least one of B2 to B4 is a heteroatom, and
(iv) the total number of heteroatoms in said heteroaryl Ring (B) is 1 to 3 and wherein each substitutable B2 to B4 C is optionally substituted with one R21B group (and the substitution on each carbon is independent of the substitutions on the remaining carbons);
R3 is selected from the group consisting of: aryl-, heteroaryl-, cycloalkyl-, cycloalkenyl, cycloalkylalkyl-, heterocyclyl-, heterocyclenyl-, heterocyclylalkyl-, heterocyclyalkenyl-, fused benzocycloalkyl-, fused benzoheterocycloalkyl-, fused heteroarylcycloalkyl-, fused heteroarylheterocycloalkyl-, fused cycloalkylaryl, fused heterocycloalkylaryl-, fused cycloalkylheteroaryl-, fused heterocycloalkylheteroaryl-, 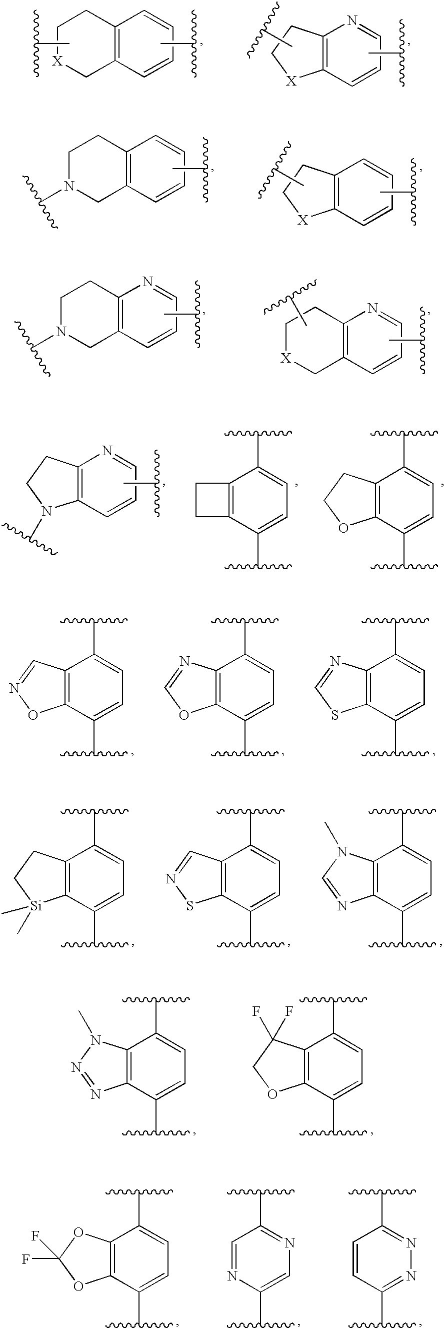 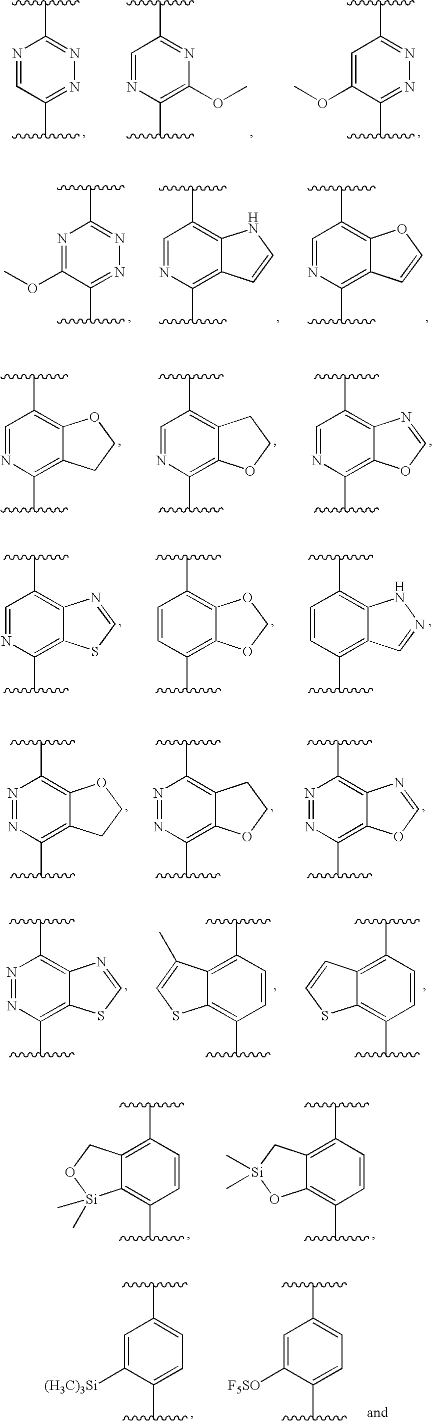 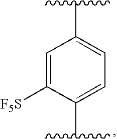
wherein X is selected from the group consisting of: O, —N(R14)- and —S—; and wherein each of said R3 moieties is optionally substituted with 1-5 independently selected R21 groups;
R4 is selected from the group consisting of: arylalkoxy-, heteroarylalkoxy-, arylalkylamino-, heteroarylalkylamino-, aryl, heteroaryl, cycloalkyl-, cycloalkenyl, heterocyclyl, heterocyclenyl, and heterocyclyalkyl-, wherein each of said R4 arylalkoxy-, heteroarylalkoxy-, arylalkylamino-, heteroarylalkylamino-, aryl, heteroaryl, heterocyclyl, heterocyclenyl, and heterocyclyalkyl- is optionally substituted with 1-5 independently selected R21 groups; or
R3 and R4 are linked together to form a fused tricyclic ring system wherein R3 and R4 are as defined above and the ring linking R3 and R4 is an alkyl ring, or a heteroalkyl ring, or an aryl ring, or a heteroaryl ring, or an alkenyl ring, or a heteroalkenyl ring;
R5 is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R15, —C(O)OR15, —C(O)N(R15)(R16), —S(O)N(R15)(R16), —S(O)2N(R15)(R16), —C(═NOR15)R16, and —P(O)(OR15)(OR16); or
R5 taken together with R1 and the nitrogen to which they are bound form a heterocycloalkyl or heterocycloalkenyl ring fused to said R1 ring, said fused ring is optionally substituted with 1 to 5 independently selected R21 groups;
R6 and R7 are each independently selected from the group consisting of: H, alkyl, alkenyl, alkynyl, aryl, arylalkyl-, alkylaryl-, cycloalkyl, cycloalkylalkyl-, heteroaryl, heteroarylalkyl-, heterocyclyl and heterocyclylalkyl-, wherein independently each of said alkyl, alkenyl and alkynyl, aryl, arylalkyl-, alkylaryl-, cycloalkyl, cycloalkylalkyl-, heteroaryl, heteroarylalkyl-, heterocyclyl and heterocyclylalkyl- is optionally substituted with 1 to 5 independently selected R21 groups; or
R6 taken together with R1 and the carbon to which they are bound form a cycloalkyl, cycloalkenyl, heterocycloalkyl or heterocycloalkenyl ring fused to said R1 ring, said fused ring is optionally substituted with 1 to 5 independently selected R21 groups; or
R6 and R7 taken together with the carbon to which they are bound form a spirocycloalkyl ring, a spirocycloalkenyl ring, a spiroheterocycloalkyl ring, or a spiroheterocyclalkenyl ring, and wherein the spiro ring is optionally substituted with 1-5 independently selected R21 groups;
R15A and R16A are independently selected from the group consisting of alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R18)q-alkyl,
(R18)q-cycloalkyl, (R18)q-cycloalkylalkyl, (R18)q-heterocyclyl, (R18)q-heterocyclylalkyl, (R18)q-aryl, (R18)q-arylalkyl, (R18)q-heteroaryl and (R18)q-heteroarylalkyl, wherein q is 1 to 5 and each R18 is independently selected;
R15, R16 and R17 are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R18)q-alkyl,
(R18)q-cycloalkyl, (R18)q-cycloalkylalkyl, (R18)q-heterocyclyl, (R18)q-heterocyclylalkyl, (R18)q-aryl, (R18)q-arylalkyl, (R18)q-heteroaryl and (R18)q-heteroarylalkyl, wherein q is 1 to 5 and each R18 is independently selected; or
each R18 is independently selected from the group consisting of alkyl, alkenyl, alkynyl, aryl, arylalkyl, arylalkenyl, arylalkynyl, —NO2, halo, heteroaryl, HO-alkyoxyalkyl, —CF3, —CN, alkyl-CN,
—C(O)R19, —C(O)OH, —C(O)OR19, —C(O)NHR20, —C(O)NH2, —C(O)NH2-C(O)N(alkyl)2, —C(O)N(alkyl)(aryl), —C(O)N(alkyl)(heteroaryl), —SR19, —S(O)2R20, —S(O)NH2, —S(O)NH(alkyl), —S(O)N(alkyl)(alkyl), —S(O)NH(aryl), —S(O)2NH2, —S(O)2NHR19, —S(O)2NH(heterocyclyl), —S(O)2N(alkyl)2, —S(O)2N(alkyl)(aryl), —OCF3, —OH, —OR20, —O-heterocyclyl, —O-cycloalkylalkyl, —O-heterocyclylalkyl, —NH2, —NHR20, —N(alkyl)2, —N(arylalkyl)2, —N(arylalkyl)-(heteroarylalkyl), —NHC(O)R20, —NHC(O)NH2, —NHC(O)NH(alkyl), —NHC(O)N(alkyl)(alkyl), —N(alkyl)C(O)NH(alkyl), —N(alkyl)C(O)N(alkyl)(alkyl), —NHS(O)2R20, —NHS(O)2NH(alkyl), —NHS(O)2N(alkyl)(alkyl), —N(alkyl)S(O)2NH(alkyl) and —N(alkyl)S(O)2N(alkyl)(alkyl); or alternately, two R18 moieties on adjacent carbons can be linked together to form: 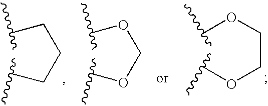
R19 is alkyl, cycloalkyl, aryl, arylalkyl or heteroarylalkyl;
R20 is alkyl, cycloalkyl, aryl, halo substituted aryl, arylalkyl, heteroaryl or heteroarylalkyl;
each R21 group is independently selected from the group consisting of alkyl, alkenyl, alkynyl, cycloalkyl, cycloalkylalkyl, cycloalkenyl, heterocyclyl (i.e., heterocycloalkyl), heterocyclylalkyl, aryl, arylalkyl, heteroaryl, heteroarylalkyl, halo,
—CN, —OR15, —C(O)R15, —C(O)OR15, —C(O)N(R15)(R16), —P(O)(CH3)2, —SO(═NR15)R16-, —SF5, —OSF5, —Si(R15A)3 wherein each R15A is independently selected, —SR15, —S(O)N(R15)(R16), —CH(R15)(R16), —S(O)2N(R15)(R16), —C(═NOR15)R16, —P(O)(OR15)(OR16), —N(R15)(R16), -alkyl-N(R15)(R16), —N(R15)C(O)R16, —CH2-N(R15)C(O)R16, —CH2-N(R15)C(O)N(R16)(R17), —CH2-R15, —CH2N(R15)(R16), —N(R15)S(O)R16A, —N(R15)S(O)2R16A, —CH2-N(R15)S(O)2R16A, —N(R15)S(O)2N(R16)(R17), —N(R15)S(O)N(R16)(R17), —N(R15)C(O)N(R16)(R17), —CH2-N(R15)C(O)N(R16)(R17), —N(R15)C(O)OR16, —CH2-N(R15)C(O)OR16, —S(O)R15A, ═NOR15, —N3, —NO2, —S(O)2R15A, —O—N═C(R15)2 (wherein each R15 is independently selected), and —O—N═C(R15)2 wherein said R15 groups are taken together with the carbon atom to which they are bound to form a 5 to 10 membered ring and wherein said ring optionally contains 1 to 3 heteroatoms independently selected from the group consisting of —O—, —S—, —S(O)—, —S(O)2-, and —NR21A;
each R21A is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —OR15, —CN, -alkyl-(R15)(R16), —CH(R15)(R16), —CH2-N(R15)C(O)R16, —CH2-N(R15)C(O)N(R16)(R17),
—CH2-R15; —CH2N(R15)(R16), —C(O)R15, —C(O)OR15, —C(O)N(R15)(R16), —C(═NOR15)R16, —CH2-N(R15)S(O)2R16A, —CH2-N(R15)C(O)N(R16)(R17), —CH2-N(R15)C(O)OR16, —C(R15)=NOR16, —S(O)R15A; —S(O)(OR15), —S(O)2(OR15), —S(O)2R15A, —S(O)N(R15)(R16), —S(O)2N(R15)(R16), —P(O)(OR15)(OR16), —N(R15)(R16), —N(R15)C(O)R16, —N(R15)S(O)R16A, —N(R15)S(O)2R16A, —N(R15)S(O)2N(R16)(R17), —N(R15)S(O)N(R16)(R17), —N(R15)C(O)N(R16)(R17), —N(R15)C(O)OR16, —N3, —NO2, —P(O)(CH3)2, —SO(═NR15)R16-, —SF5, and —OSF5;
each R21B group is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —OR15, —CN, -alkyl-(R15)(R16), —CH(R15)(R16), —CH2-N(R15)C(O)R16, —CH2-N(R15)C(O)N(R16)(R17), —CH2-R15, —CH2N(R15)(R16), —C(O)R15, —C(O)OR15, —C(O)N(R15)(R16), —C(═NOR15)R16, —CH2-N(R15)S(O)2R16A, —CH2-N(R15)C(O)N(R16)(R17), —CH2-N(R15)C(O)OR16, —C(R15)=NOR16, —SR15; —S(O)R15A; —S(O)(OR15), —S(O)2(OR15), —S(O)2R15A, —S(O)N(R15)(R16), —S(O)2N(R15)(R16), —P(O)(OR15)(OR16), —N(R15)(R16), —N(R15)C(O)R16, —N(R15)S(O)R16A, —N(R15)S(O)2R16A, —N(R15)S(O)2N(R16)(R17), —N(R15)S(O)N(R16)(R17), —N(R15)C(O)N(R16)(R17), —N(R15)C(O)OR16, —N3, —NO2, —P(O)(CH3)2, —SO(═NR15)R16-, —SF5, —OSF5, and —Si(R15A)3 wherein each R15A is independently selected;
independently, each alkyl, cycloalkenyl, cycloalkyl, cycloalkylalkyl, heterocyclyl, heterocyclylalkyl, aryl, arylalkyl, heteroaryl, heteroarylalkyl, alkenyl and alkynyl R21, R21A, and R21B group is optionally substituted by 1 to 5 independently selected R22 groups wherein each R22 group is independently selected from the group consisting of alkyl, cycloalkyl, cycloalkenyl, heterocyclyl, aryl, heteroaryl, halo, —CF3, —CN, —OR15, —C(O)R15, —C(O)OR15, -alkyl-C(O)OR15, C(O)N(R15)(R16), —SR15, —S(O)N(R15)(R16),
—S(O)2N(R15)(R16), —C(═NOR15)R16, —P(O)(OR15)(OR16), —N(R15)(R16), -alkyl-N(R15)(R16), —N(R15)C(O)R16, —CH2-N(R15)C(O)R16, —N(R15)S(O)R16, —N(R15)S(O)2R16, —CH2-N(R15)S(O)2R16, —N(R15)S(O)2N(R16)(R17), —N(R15)S(O)N(R16)(R17), —N(R15)C(O)N(R16)(R17), —CH2-N(R15)C(O)N(R16)(R17), —N(R15)C(O)OR16, —CH2-N(R15)C(O)OR16, —N3, =NOR15, —NO2, —S(O)R15A and —S(O)2R15A; and
With the proviso that when R3 is aryl and R1 comprises a 5 or 6-membered aryl or heteroaryl ring, then said 5 or 6-membered aryl or heteroaryl ring is not substituted with an R21 group that is selected from the group consisting of the moieties:
—O-(5 or 6 membered aryl), —S-(5 or 6 membered aryl), —S(O)2-(5 or 6 membered aryl), —N(R15)-(5 or 6 membered aryl), —C(O)-(5 or 6 membered aryl),
-alkyl-(5 or 6 membered aryl), —O-(5 or 6 membered heteroaryl),
—S-(5 or 6 membered heteroaryl), —S(O)2-(5 or 6 membered heteroaryl),
—N(R15)-(5 or 6 membered heteroaryl), —C(O)-(5 or 6 membered heteroaryl), and
-alkyl-(5 or 6 membered heteroaryl).

57. The compound of claim 56, wherein R4 is selected from the group consisting of:
1 gg to 13 gg.

58. The compound of claim 56, wherein the R4-R3 moiety is selected from the group consisting of: 1 bb to 40 bb.

59. The compound of claim 56, wherein R1 is selected from the group consisting of:

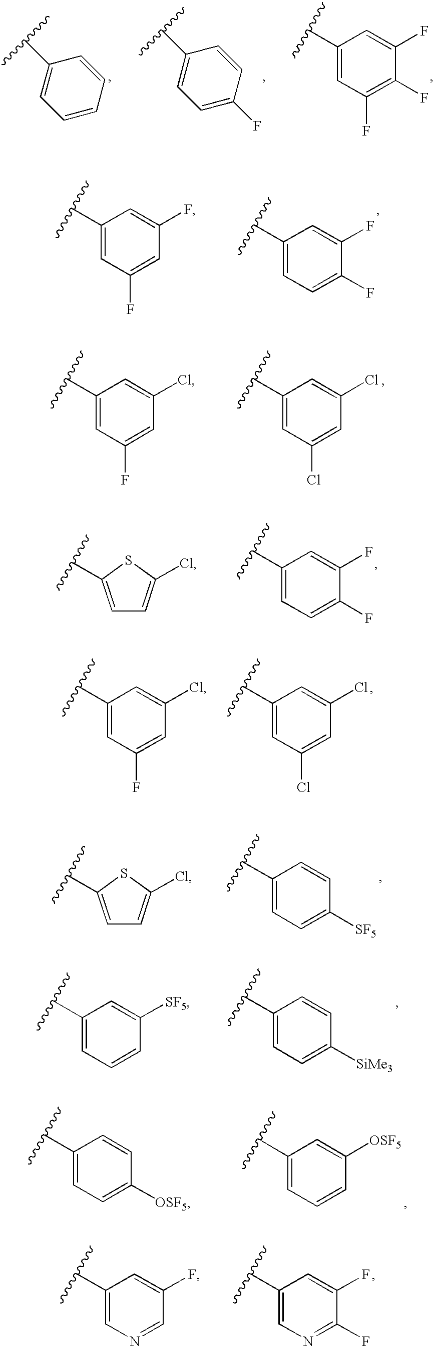

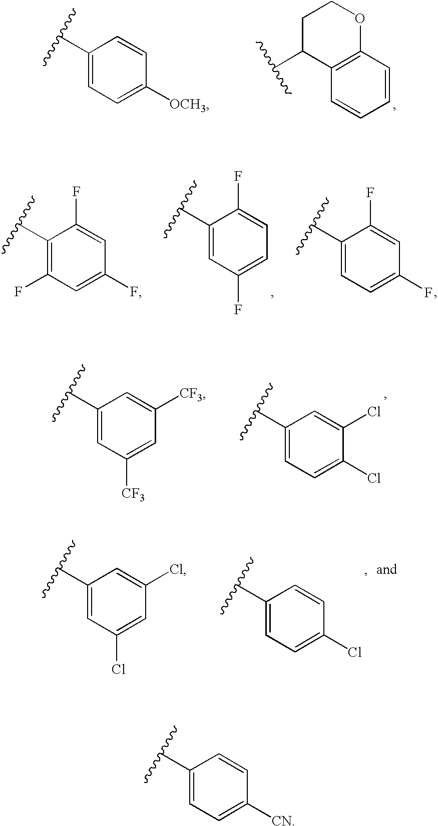

60. The compound of claim 56, wherein:
(a) R1 is phenyl substituted with 1 to 3 independently selected R21 moieties wherein at least one R21 moiety is selected from the group consisting of —SF5, —OSF5 and —Si(R15A)3; or
(b) R1 is phenyl substituted with 1 to 3 independently selected R21 moieties wherein at least one R21 moiety is selected from the group consisting of —SF5 and —OSF5.

61. The compound of claim 56, wherein L is selected from the group consisting of: 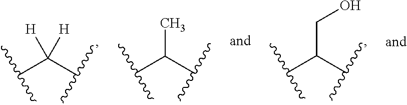
R¹ is selected from the group consisting of:

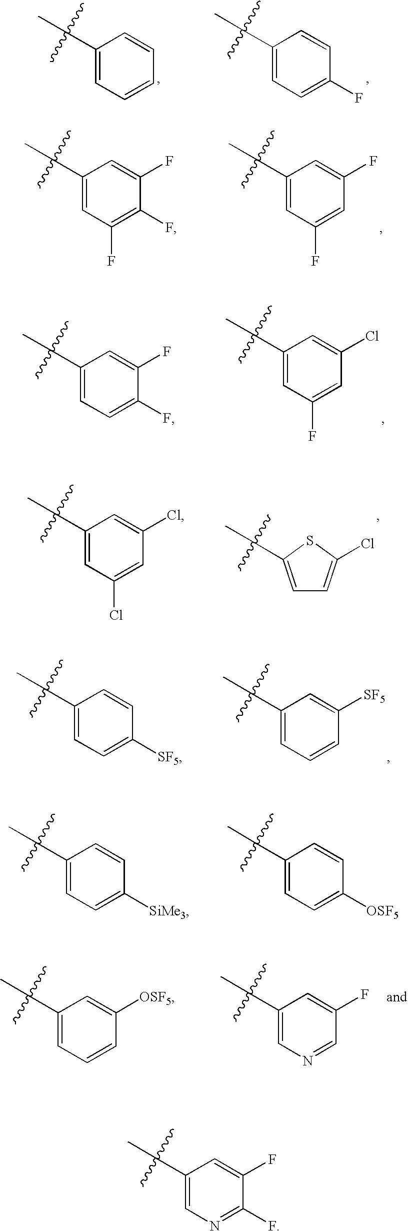

62. The compound of claim 56, wherein the R4-R3- moiety is:

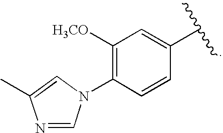

63. The compound of claim 56, wherein
(a) said R3 is selected from the group consisting of aryl and aryl substituted with 1 to 3 independently selected R21 groups, and said R9 group is selected from the group consisting of heteroaryl and heteroaryl substituted with 1 to 3 independently selected R21 groups; or
(b) said R3 is selected from the group consisting of: phenyl, and phenyl substituted with one R21 group, and (2) said R4 is imidazol-1-yl substituted with one R21 group.

64. The compound of claim 56, wherein the R4-R3- moiety is selected from the group consisting of

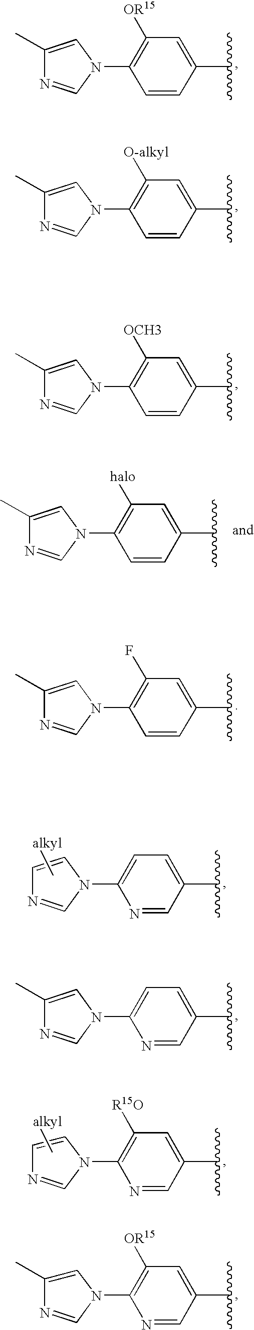

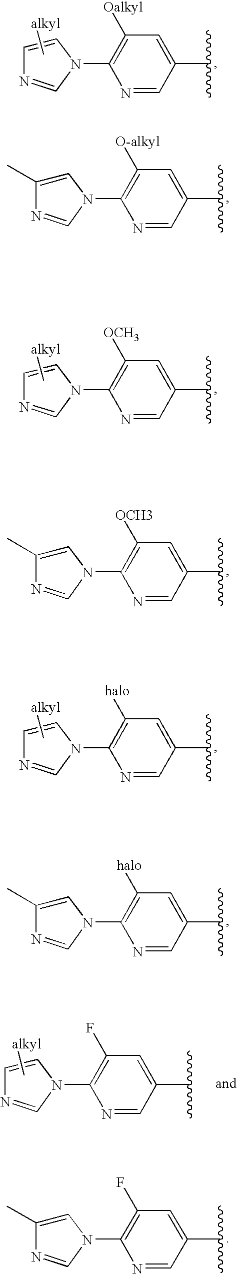

65. The compound of claim 56, wherein the -L-R1 moiety is selected from the group consisting of:

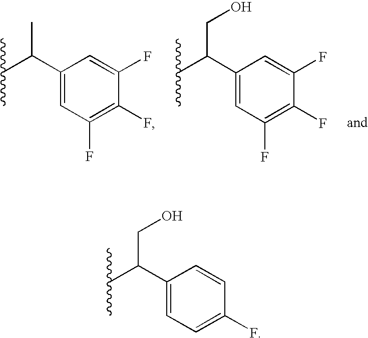

66. The compound of claim 56, wherein L is —C(R6)(R7)- wherein R6 and R7 are independently selected from the group consisting of: H and alkyl.

67. The compound of claim 56, wherein R1 is phenyl substituted with 1 to 3 independently selected R21 groups wherein said R21 groups are halo.

68. The compound of claim 56, wherein L is selected from the group consisting of: 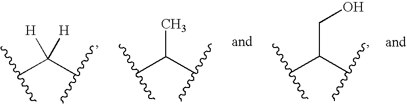
R¹ is selected from the group consisting of:

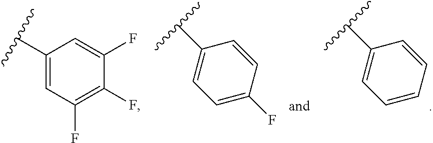

69. The compound of claim 56, wherein the -L-R1 moiety is:

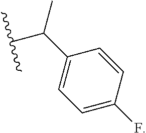

70. The compound of claim 56, wherein the fused Rings (A) and (B) are selected from the group consisting of: 1A to 4A, A1.2 to A22.2, and A24.2 to A28.2.

71. The compound of claim 56, wherein the compound of formula (I) is
(a) a compound selected from the group consisting of the compounds of formulas (IA), (IB), (IC), (ID), and (IE); or
(b) compound selected from the group consisting of the compounds 5.1, 8.1, 11.1, and A1 to A28.

72. A pharmaceutical composition comprising:
(a) a therapeutically effective amount of at least one compound of claim 56 or a pharmaceutically acceptable salt thereof, solvate, or ester thereof, and a pharmaceutically acceptable carrier;
(b) a therapeutically effective amount of at least one compound of claim 56, or a pharmaceutically acceptable salt, solvate, or ester thereof, and at least one pharmaceutically acceptable carrier, and an effective amount of one or more other pharmaceutically active drugs;
(c) a therapeutically effective amount of at least one compound of claim 56, or a pharmaceutically acceptable salt, solvate, or ester thereof, and at least one pharmaceutically acceptable carrier, and an effective amount of one or more other pharmaceutically active drugs selected form the group consisting of one or more BACE inhibitors, muscarinic antagonists, cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA inverse agonists; inhibitors of amyloid aggregation; glycogen synthase kinase beta inhibitors; promoters of alpha secretase activity; PDE-10 inhibitors and cholesterol absorption inhibitors; or
(d) a therapeutically effective amount of at least one compound of claim 56, or a pharmaceutically acceptable salt, solvate, or ester thereof, and at least one pharmaceutically acceptable carrier, and an effective amount of donepezil hydrochloride.

73. A method for treating Alzheimer's disease, the method comprising administering to a patient in need thereof a therapeutically effective amount of a compound according to claim 56 or pharmaceutically acceptable salt thereof.

74. A method for inhibiting the deposition of amyloid protein in, on or around neurological tissue, the method comprising administering to a patient in need thereof an effective amount of a compound according to claim 56 or a pharmaceutically acceptable salt thereof.
